(19) United States
(12) Patent Application Publication
(10) Pub. No.: US 2002/0061599 A1
(43) Pub. Date: May 23, 2002

(54) Method of identifying ligands of biological target molecules

(76) Inventors: Christian E. Elling, Copenhagen (DK); Birgitte Holst Lange, Copenhagen (DK); Thue W. Schwartz, Frederiksburg (DK); Lars Ole Gerlach, Copenhagen (DK); Jan Torleif Pedersen, Bronshoj (DK)

Correspondence Address: Dike, Bronstein, Roberty & Cushman, Intellectual Property Practice Group, EDWARDS & ANGELL, LLP, 130 Water Street, Boston, MA, 02109-4280, US

(21) Appl. No.: 09/752,102

(22) Filed: Dec. 29, 2000

Related U.S. Application Data
(60) Provisional application No. 60/175,664, filed on Jan. 12, 2000.
(60) Provisional application No. 60/175,401, filed on Jan. 11, 2000.
(60) Provisional application No. 60/202,990, filed on May 9, 2000.

(30) Foreign Application Priority Data
Dec. 30, 1999 (DK) DK 199901879
Dec. 30, 1999 (DK) DK 199901880
Apr. 28, 2000 (DK) DK 200000705

(51) Int. Cl. G01N 33/53; G01N 33/543

(52) U.S. Cl. 436/518; 435/7.1

(57) ABSTRACT

The present invention provides a molecular approach for rapidly and selectively identifying small organic molecule ligands, i.e. compounds, that are capable of interacting with and binding to specific sites on biological target molecules. The methods of the present invention are applicable to any biological target molecule that has or can be manipulated to have a metal-ion binding site. Biological target molecules are e.g. proteins, polypeptides, oligopeptides, nucleic acids, carbohydrates, nucleoproteins, glycoproteins, glycolipids, lipoproteins and derivatives thereof. More specifically, the biological target molecules include membrane receptors, signal transduction proteins, scaffolding proteins, nuclear receptors, steroid receptors, intracellular receptors, transcription factors, enzymes, allosteric enzyme regulatory proteins, growth factors, hormones, neuropeptides and immonoglobulins. A very interesting group of biological target molecules are membrane proteins such as, e.g., transmembrane protein (e.g. 7 TMs).

The methods described herein make it possible to construct and screen libraries of compounds specifically directed against predetermined epitopes on the biological target molecules. The compounds are initially constructed to be bi-functional, i.e. having both a metal-ion binding moiety, which conveys them with the ability to bind to either a natural or an artificially constructed metal-ion binding site as well as a variable moiety, which is varied chemically to probe for interactions with specific parts of the biological target molecule located spatially adjacent to the metal-ion binding site. Compounds may subsequently be further modified to bind to the unmodified biological target molecule without help of the bridging metal-ion. The methods according to the invention may be performed easily and quickly and lead to unambiguous results. The compounds identified by the methods described herein may themselves be employed for various applications or may be further derivatised or modified to provide novel compounds.

### FIELD OF THE INVENTION

The present invention relates to a novel method useful for identifying small organic molecule ligands (in the following also denoted “compounds”) for binding to specific sites on biological target molecules such as proteins, nucleic acids, carbohydrates, nucleoproteins, glycoproteins and glycolipids. The compounds are capable of interacting with the biological target molecule, in particular with a protein, in such a way as to modify the biological activity thereof.

The invention further relates to methods of identifying compounds acting as ligands of biological target molecules such as, e.g., proteins involving the introduction of metal ion binding sites into the biological target molecules, including a method of identifying compounds that bind to orphan receptors. Small organic ligands identified according to the methods of the present invention find use, for example, as novel therapeutic drug compounds or drug lead compounds, enzyme inhibitors, labelling compounds, diagnostic reagents, affinity reagents e.g. for protein purification etc.

### INTRODUCTION

The initial phase in developing novel biologically active compounds such as, e.g., therapeutically or propylactically active drug compounds is to identify and characterize one or more binding ligand(s) for a given biological target. Many molecular techniques have been developed and are currently being employed for identifying novel ligands or compounds that bind to the biological target. In the following proteins are used as an example on a biological target molecule,

Proteins as drug targets

Most drug compounds act by binding to and altering the function of proteins. These can be intracellular proteins such as, for example enzymes and transcription factors, or they can be extracellular proteins, for example enzymes, or they can be membrane proteins. Membrane proteins constitute a numerous and varied group whose function is either structural, for example being involved in cell adhesion processes, or the membrane proteins are involved in intercellular communication and communication between the cell exterior and the interior by transducing chemical signals across cell membranes, or they facilitate or mediate transport of compounds across the lipid membrane. Membrane proteins are for instance receptors and ion channels to which specific chemical messengers termed ligands bind resulting in the generation of a signal, which gives rise to a specific intracellular response (this process is known as signal transduction). Membrane proteins can, for example also be enzymes which are associated to the membrane for functional purposes, e.g. proximity to their substrates. Most membrane proteins are anchored in the cell membrane by a sequence of amino acid residues, which are predominantly hydrophobic to form hydrophobic interactions with the lipid bilayer of the cell membrane. Such membrane proteins are also known as integral membrane proteins. In most cases, the integral membrane proteins extend through the cell membrane into the interior of the cell, thus comprising an extacellular domain, one or more transmembrane domains and an intracellular domain. A large fraction of current drugs act on membrane proteins and among these the majority are targeted towards the G protein coupled receptors (GPCR) with their seven transmembrane segments, also called 7TM receptors.

Identification of lead compounds in drug discovery

Drug discovery traditionally involves a process where a lead compound first is identified and then subsequently chemically optimised for high affinity and selectivity for the protein target (or another biological target molecule) and optimised for other drug-like properties such as lack of toxic effects and desirable pharmacokinetics.

Recent drug development has focused on screening of large libraries of chemical compounds in order to identify lead compounds, which are capable of either upregulating (called agonists) or downregulating the activity of the protein target (called antagonists), as required. Screening has usually been performed in a “shot-gun” fashion by setting up an assay for screening large numbers of compounds, e.g. large files of compounds or compounds in combinatorial libraries, in order to identify compounds with the desired activity. The subsequent chemical optimization of the lead compounds obtained from such screening procedures has been performed very much in a trial-and-error fashion and has been quite cumbersome and resource-demanding, involving procedures such as described by E. Sun and F. E. Cohen, Gene 1993 137(1), 127-32, or J. Kuhlmann, Int J Clin Pharmacol Ther. 1999 37(12), 575-83. A major disadvantage of the drug discovery process is that it is difficult to identify active compounds with sufficient selectivity and specificity for a given target protein or in many cases it is even difficult at all to identify suitable lead compounds, for example for interfering with protein-protein interactions.

Optimization of lead compounds to high affinity ligands

Through the generation of chemical analogs of the lead compound and testing of these for binding or activity on the biological target molecules such as a protein target, the lead compound is gradually improved in affinity for the target. Also this process in to a large degree done by trial-and-error, although the medicinal chemist usually is guided by a gradually increasing knowledge in the structure activity relationship (SAR) of the compounds, i.e. the observation of which modification at which site in the compound that increase or decrease the activity of the compound. The SAR can provide a great deal of information regarding the nature of ligand-receptor interactions, but no detailed information about the location and actual chemical nature of the binding site in the target protein is provided. A number of closely related chemical structures are used to direct the orientation of the ligand within the putative binding cavity and to determine what part of the ligand is involved in binding to the receptor. This technique has its limitations due to the fact that changing the structure of the ligand may result in a actual change in the binding site of the receptor (Mattos et al. Struct. Biol., 1995 1:55-58), a fact which obviously still would be un-know to the medicinal chemist. Thus, in most cases the lack of knowledge of tee precise molecular interaction with the receptor of the lead compounds found by chemical screening has prevented a rational chemical approach to the optimisation of the lead compound.

Identification of ligand binding sites

Determination of the three-dimensional structure of the target protein either alone or even better in complex with the ligand by X-ray crystallography provides high-resolution and very high quality information about the molecular recognition of the compound in the target protein structure. In the case, where the target is a soluble protein it is often possible to perform rationalized lead compound optimization through crystallisation of the lead compound in complex with the target protein, analyse the molecular interactions and identity possible ways of improving these interactions and on this basis new compounds with improved affinity are synthesised. Subsequent X-ray analysis of complexes of these improved compounds and the target protein can then lead to the synthesis of a new series of further improved compounds, new compound-target crystalisations and so on until the desired affinity has been obtained.

However, these methods of structure based lead compound optimization or “rational drug discovery” can only be applied to soluble proteins, which are relatively easy to crystallise. For example, membrane proteins which constitute a majority of drug targets are very difficult or in most cases still impossible to crystallise. A variety of methods have been employed in order to characterize ligand-receptor interactions in proteins where three-dimensional structures cannot be obtained, For example, site-directed mutagenesis is used to eliminate a ligand binding site or part of a ligand binding site by substitution of selected amino acid residues with other residues, e.g. alanine. Only a few cases have been presented where ligand binding sites have been thoroughly investigated by an extensive and systematic mutational analysis of all possible residues in a given area ad with combination of both mutational analysis of the receptor and chemical analysis of the ligand (e.g. the β-adrenergicreceptor, Strader et al., FASEB J. 3, 1989, pp. 1825-1832; Strader et al, J. Biol. Chem. 266(1), 1991, pp. 5-8; Schambye et al, Mol. Pharm., 1995 47:425-431).

A general problem of the site-directed mutagenesis method is that it is not clear whether the substitution of a residue affects the binding of a ligand directly (i.e. the residue is directly involved in ligand binding) or indirectly (i.e. the residue is only involved in the structure of the receptor). Another problem of Ala substitution is false negative results because the procedure basically creates another “hole” in the presumed binding pocket through removal of the side chain on the residue replaced by Ala. The effect of Ala substitution is highly dependent on the relative contribution to the binding energy of the replaced residue. An alternative, to Ala substitution is steric hindrance mutagenesis where for example a larger side chain, e.g. Trp, are introduced in a presumed binding pocket as described by Holst et al., Mol Pharmacol. 53(1), 1998, pp. 166-175.

Methods such as photoaffinity labelling has also been proven to be a useful tool in identifying domains of receptors involved in ligand binding (Dohlman et al., Ann. Rev. Biochem. 60, 1991, pp. 653-688) A photoreactive group is attached or built into the ligand. After binding, the ligand-receptor complex is exposed to UV light, resulting in crosslinking of the ligand to the receptor. Finally the complex is digested with proteases and the ligand-binding part of the receptor can be identified. It should be noted however, that except for proteins where crystal- or NMR-structures can be made, it is only in a few cases where binding pockets for ligands in fact have been identified with a reasonable degree of accuracy. This is especially the case for membrane proteins. In even fewer cases have the actual pattern of chemical recognition been determined well in these proteins, i.e. identification of which chemical moiety of the ligand interacts with which side-chain or with which part of the backbone in the target (Schwartz et al. Current Opin. Biotechnol., 1994 4:434-444). In the very few cases of for example membrane proteins where some information is available concerning the presumed binding pocket or perhaps even about actual chemical interactions, this is only the case for final, high-affinity optimized drugs. No information along these lines are today known for lead compounds found by chemical screening in for example membrane proteins. Even in the case where an X-ray structure is known for a complex between a compound or a drug and its target protein, it is often not possible to predict the binding mode of close analogs of this since modification of the compound may seriously alter the overall binding mode involving also parts of the compound which have not been chemically modified (Mattos et al. Struct. Biol., 1995 1:55-58). Thus, a chemical “anchor”, i.e. a well identified binding point between a chemical moiety in the compound and a particular site in the target protein, would be highly beneficial in order to efficiently apply structure based drug discovery techniques to both proteins with known three dimensional structures and to protein targets for which meaningful molecular models can be built based on homology to known protein structures.

The present invention deals with methods involving a chemical “anchor” by making use of a metal binding site in the target biological molecule as well a metal binding site in a chemical compound. The metal binding site in the biological target molecule such as, e.g., a target protein may be a natural metal-ion binding site or it may be a metal-ion binding site that has been introduced into the protein by artificial means such as, e.g., engineering means.

### BACKGROUND OF THE INVENTION

Natural metal-ion sites in proteins

Many proteins contain metal-ion binding sites. These metal-ion sites serve either structural purposes, for example stabilizing the three-dimensional structure of the protein, or they serve functional purposes, where the metal-ion may for example be part of the active site of an enzyme. It is well known that also several integral membrane proteins include binding sites for metal ions. The coordination of metal ions to metal ion binding sites is well characterized in numerous high-resolution X-ray and NMR structures of soluble proteins; for example, distances from the chelating atoms to the metal ion as well as the preferred conformation of the chelating side chains are known (e.g. J. P. Glusker, Adv. Protein Chem. 42, 1991, pp. 3-76; P. Chaklrabarty, Protein Eng. 4, 1990, pp. 57-63; R. Jerigan et al., Curr. Opin. Struct. Biol. 4, 1994, pp 256-263). Thus, metal-ion binding in proteins is one of the most well characterised forms of ligand-protein interactions known. Hence, characterising a metal ion-binding site in a membrane protein using, for example, molecular models and site directed mutagenesis can yield information about the structure of the membrane protein and importantly where the “ligand” (metal ion) binds (e.g. Elling et al. Fold. Des. 2(4), 1997, pp. S76-80).

Metal-ion site engineering in proteins

Engineering of artificial metal ion binding sites into membrane proteins has been employed to explore the structure and function of these proteins. Thus, C. E. Elling et al., Nature 374, 1995, pp. 74-77, have reported how the binding site for a proto-type antagonists for the tachykinin NK-1 receptor could be converted into a metal ion-binding site by systematic substitution of residues in the binding pocket with His residues. If side chains of amino acid residues participating in metal ion binding are known, it imposes a distance constraint on the protein structure which can be used in the interpretation of unknown protein structures (C. E. Elling and T. W. Schwartz, EMBO J. 15(22), 1996, pp. 6213-6219; C. E. Elling et al., Fold. Des. 2(4), 1997, pp. S76-80). Recently the generation of an activating metal ion binding site has been reported for the β₂-adrenergic receptor, where the binding site for the normal catecholamine ligands was exchanged with a metal-ion site through specific substitutions in the binding pocket for the agonists (C. E. Elling et al, PNAS 96, 1999, pp. 12322-12327). This metal-ion binding site could be addressed also with metal-ions in complex with metal-ion chelators, i.e. small organic compounds binding metal-ions.

However, none of the above-mentioned documents address the concept of using a chemical “anchor” in the drug discovery process.

### SUMMARY OF THE INVENTION

The present invention provides a molecular approach for rapidly and selectively identifying small organic molecule ligands, i.e. compounds, that are capable of interacting with and binding to specific sites on biological target molecules. The methods described herein make it possible to construct and screen libraries of compounds specifically directed against predetermined epitopes, on the biological target molecules. The compounds are initially constructed to be bi-functional, i.e. having both a metal-ion binding moiety, which conveys them with the ability to bind to either a natural or an artificially constructed metal-ion binding site as well as a variable moiety, which is varied chemically to probe for interactions with specific parts of the biological target molecule located spatially adjacent to the metal-ion binding site. Compounds may subsequently be further modified to bind to the unmodified biological target molecule without help of the bridging metal-ion. The methods according to the invention may be performed easily and quickly and lead to unambiguous results The compounds identified by the methods described herein may themselves be employed for various applications or may be further derivatised or modified to provide novel compounds.

The methods of the present invention are applicable to any biological target molecule that has or can be manipulated to have a metal-ion binding site. However, in the following proteins are used as examples of biological target molecules.

Parts of the present invention utilise the finding that many proteins in their natural form posses a metal-ion binding site, which may or may not have been record previously, However, in order to obtain a general applicability of the technology to a broad range of biological target molecules, the invention especially utilizes the possibility to mutate proteins, for example a receptor, an enzyme or a transcriptional regulator in such a way, that they comprise a metal ion binding site. The metal-ion site is then used as an anchor-point for the initial parts of the medicinal chemistry drug-discovery process, during which test compounds ran be synthesized, which due to their specific interaction with the metal-ion binding site can be deliberately directed towards interaction with specific, functionally interesting parts of the biological target molecule. The test compounds are subsequently structurally optimised for interaction with spatially neighboring parts of the proteins (that is, interaction with the side chains or backbone of one or more neighbouring amino acid residues). These compounds can then be utilized as leads or starting points for the construction of ligands binding to the wild-type protein. In this way it is possible to predetermine the binding site of a compound to a particular location in a protein structure and thereby target the optimised compounds to sites where binding of the compound will alter the biological activity of the protein in a desired way, for example to increase or decrease its biological activity. By selecting the binding site for a test compound at will and thereby selecting the binding site for the optimised compound (such as a drug candidate) in a protein, it is for example possible to:

1) speed up the process of development of high affinity drug candidates or other compounds with biological activity because a more efficient structure-based compound optimisation process can be applied;

2) obtain high selectivity for a given member of a protein family by targeting the compound to a site in the protein which differs between different members of the protein family;

3) obtain new functionalities of compounds by targeting them to allosteric modulatory sites in proteins.

These constitute some of the advantages of the present invention.

In the course of research leading to the present invention, the inventors have found that certain small organic compounds which bind metal ions (i.e. metal ion chelators) are also able to bind to metal ion binding sites in various proteins, including membrane proteins for example receptors, in such a way that the metal ion acts as a bridge between the small organic compound and the protein. Importantly, the present invention has made it possible to predetermine or identify and localise the exact binding site and binding mode of such metal ion chelates used as test compounds, contrary to what has been known in the art for test compounds in general. Based on the identification or confirmation of the binding site of the test compounds, using for example site-directed mutagenesis, three-dimensional structure determination by for example X-ray crystallography or NMR or molecular models of the protein and techniques such as those described above, a rational approach may be taken to the chemical optimisation of the test compounds. Thus, relatively small chemical libraries may be made, the compounds in which may be designed to interact with specific amino acid residues of the protein in question. Compounds that exhibit a high affinity binding to the protein and affect the biological activity of the protein in a desired way may then be selected for further optimisation.

The metal-ion binding portion of the test compounds may subsequently be removed or altered to no longer posses metal-ion binding properties, and the test compounds, as well as chemical derivatives thereof may be constructed to interact with side chains of other amino acids in the vicinity of the artificial metal ion binding site, and tested for binding to the wild-type protein which does not include a metal ion binding site. Accordingly, relatively small chemical libraries may be made, the compounds in which may be designed to interact with the specific amino acid residues found in the wild-type protein at or spatially surrounding the location where the metal ion site had initially been engineered.

Thus, the present invention is based on the general principle, applicable to any biological target molecule including a protein, of introducing metal ion binding sites at any position in e.g. the protein where a test compound binding to the protein is likely to exert an effect on the biological activity of the protein. This may for example be 1) at a site where the test compound will interfere with the binding to another protein, for example a regulatory protein, or to a domain of the same protein; 2) at a site where the binding of the test compound will interfere with the cellular targeting of the protein; 3) at a site where the binding of the test compound will directly or indirectly interfere with the binding of substrate or the binding of an allosteric modulatory factor for the protein; 4) at a site where the binding of the test compound may interfere with the intra-molecular interaction of domains within the protein, for example the interaction of a regulatory domain with a catalytic domain; 5) at a site where binding of the test compound will interfere with the folding of the protein, for example the folding of the protein into its active conformation; or 6) at a site which will interfere with the activity of the protein, for example by an allosteric mechanism. Subsequent to identifying test compounds that bind to the artificial metal ion binding site of the protein, information may be acquired of the structure of the binding site and of amino acid residues in its immediate vicinity. Such information may be used in the design of compounds with improved binding affinity to the proteins resulting from interaction with one or more amino acid residues in the vicinity of the metal ion binding site. Such compounds may, in turn, be used in the design of potential drug candidates or other compounds with a desired activity on the corresponding wild-type, non-mutated protein.

Accordingly, the present invention relates to a drug discovery process for identification of a small organic compound that is able to bind to a biological target molecule, the process comprising mutating a biological target molecule in such a way that at least one amino acid residue capable of binding a metal ion is introduced into the biological target molecule so as to obtain a metal ion binding site as an anchor point in the mutated biological target molecule.

The mutated biological target molecule may furthermore be contacted with a test compound which comprises a moiety including at least two heteroatoms for chelating a metal ion, under conditions permitting non-covalent binding of the test compound to the introduced metal ion binding site of the mutated biological target molecule, and then followed by detection of any change in the activity of the mutated biological target molecule or determination of the binding affinity of the test compound to the mutated biological target molecule.

The present invention relates also to a drug discovery process for identification of a small organic compound that is able to bind to a biological target molecule which has at least one metal ion binding site, the process comprising

(a) contacting the biological target molecule with a test compound which comprises a moiety including at least two heteroatoms for chelating a metal ion, under conditions permitting non-covalent binding of the test compound to the metal ion binding site of the biological target molecule, and

(b) detecting any change in the activity of the biological target molecule or determining the binding affinity of the test compound to the biological target molecule.

A very important class of biological target molecules amenable to testing according to the present invention are proteins such as membrane proteins which includes proteins that are involved in intercellular communication and other biological processes of profound importance for cellular activity. Thus, in another aspect, the present invention relates to a method of identifying a metal ion binding site in a protein, the method comprising

(a) selecting a nucleotide sequence suspected of coding for a protein and deducing the amino acid sequence thereof,

(b) expressing said nucleotide sequence in a suitable host cell,

(c) contacting said cell or a portion thereof including the expressed protein with a test compound which comprises a moiety including at least two heteroatoms for chelating a metal ion, under conditions permitting non-covalent binding of the test compound to the protein, and detecting any change in the activity of the protein or determining the binding affinity of the test compound to the protein, and

(c) determining, based on the generic three-dimensional model of the class of proteins to which the protein or suspected protein belongs, at least one metal ion binding amino acid residue located in said protein to locate the metal ion binding site of said protein.

In a still further aspect the invention relates to a method of mapping a metal on binding site of a protein, it method comprising

(a) contacting the protein with a test compound which comprises a moiety including at least two heteroatoms for chelating a metal ion, under conditions permitting non-covalent binding of the test compound to the protein, and detecting any change in the activity of the protein or determining the binding affinity of the test compound to the protein, and

(b) determining, based on the primary structure of the specific protein in question and the generic three-dimensional model of the class of proteins to which the specific protein of step (a) belongs, at least one metal ion binding amino acid residue located in the membrane protein to identify the metal ion binding site of said membrane protein.

In a further aspect the invention relates to chemical libraries comprising test compounds in chelated or non-chelated form and to a chemical library comprising metal ions suitable for chelating test compounds. The metal ions are generally presented in salt form or in the form of complexes or solvates.

In still further aspects the invention relates to the use of test compounds as tracers in binding assays for orphan receptors and in pharmacological knock-out experiments.

Further aspects of the invention as well as preferred embodiments of the invention appear from the appended claims.

The details and particulars described for e.g. the drug discovery process aspect apply mutatis mutandis—whenever relevant—to all other aspects of the invention.

### DETAILED DESCRIPTION OF THE INVENTION

Essential parts of the present invention relates to methods of identifying compounds that are capable of binding to specific sites on biological target molecules. Much of the detailed description of the invention is dealt with in the description of the examples presented in “EXPERIMENTAL”. In a typical form of this process the following steps are involved:

(1) Identification or engineering, of metal-ion binding sites to be exploited as anchor points for lead compounds—In one embodiment of the invention, the biological target molecule already has a suitable metal-ion site, which may or may not previously have been recognized. In another sore broadly applicable form of the invention such metal-ion sites are introduced, for example through mutagenesis, at specific sites in the biological target molecule expected to be useful as anchor points for the development of compounds affecting the function of the target molecule in a desired way. In one form of the invention a number of such sites are introduced and one or more are selected for further use.

(2) Selection of lead compound from library of metal ion chelating compounds—Basic libraries of metal-ion chelators exposing a systematic range of chemical moieties differing in potential chemical interaction-mode with the surrounding parts of the biological target molecule are screened for lead or test compounds which will bind to the metal-ion site in the biological target molecule and affect its function in a desired way.

(3) Chemical optimisation of lead compound for secondary interaction points In the biological target molecule—Based on the selected lead compound, libraries of basic bi-functional compounds are being constructed in which the compounds have both a anchoring metal-ion binding moiety, which conveys them with the ability to bind to the metal-ion binding site in the biological target molecule, as well as a variable moiety, which is varied chemically to probe for improved interactions with specific parts of the biological target molecule located spatially adjacent to the metal-ion binding site. In one preferred form of the invention these libraries are constructed based on structural knowledge of the chemical target moiety in the biological target molecule. In another form a more broad screening of larger libraries of compounds is performed without detailed knowledge of the structure of the biological target molecule surrounding the anchoring metal-ion site.

(4) Chemical optimisation of lead compound for high affinity interaction with wild type biological target molecule—exchange of metal ion anchor with “ordinary” chemical interaction—When a compound has been developed having a suitable, detectable affinity also on the wild-type form of the biological target molecule usually without metal-ion present, then this compound is further optimized for high affinity binding and effect on the wild-type molecule. In one form of the invention structure-based construction of chemical libraries will be performed in order to take advantage of the possibility to directly exchange the metal-ion bridge with other types of chemical interactions with the amino acid residues found in the wild type molecule.

The present invention is directed to methods directly or indirectly involved in the above-mentioned drug discovery process. Furthermore, it is directed to the use of chemical libraries and to a method for selecting a chemical compound from a library.

The following detailed description of the invention is mainly concerned with methods of identifying compounds interacting with proteins such as, e.g., membrane proteins. It should be understood, however, that the discussion of the detailed method steps apply equally to other biological target molecules like nucleic acids, carbohydrates, nucleoproteins, glycoproteins and glycolipids.

In the following some definitions are first dealt with following by a detailed description of the invention according to the four main steps of the drug discovery process:

### DEFINITIONS

Throughout the text including the claims, the following terms shall be defined as indicated below.

A “test compound” is intended to indicate a small organic molecule ligand or a small organic compound which is capable of interacting with a biological target molecule, in particular with a protein, in such a way as to modify the biological activity thereof. The term includes in its meaning metal ion chelates of the formulas shown below. Furthermore, the term includes in its meaning metal ion chelates of the formulas shown below as well as chemical derivatives thereof constructed to interact with other part(s) of the biological target molecule than the metal ion binding site. In proteins such an interaction may take place with side chains of amino acids or amino acid residues in the vicinity of the natural or artificial metal ion binding site. A test compound may also be an organic compound which in its structure includes a metal atom via a covalent binding. Such test compounds will generally contain at least one heteroatom such as, e.g., N, O, S, Se and/or P.

A “metal ion chelator” is intended to indicate a compound capable of forming a complex with a metal atom or ion. Such a compound will generally contain a heteroatom such as N, O, S, Se or P with which the metal atom or ion is capable of forming a complex.

A “metal ion chelate” is intended to indicate a complex of a metal ion chelator and a metal atom or ion.

A “metal ion binding site” is intended to indicate a part of a biological target molecule which comprises an atom or atoms capable of complexing with a metal atom or ion. Such an atom will typically be a heteroatom, in particular N, O, S, Se or P. With respect to proteins a metal ion binding site is typically an amino acid residue of the protein which comprises an atom capable of complexing with a metal ion. These amino acid residues are typically but nor respricted to histidine, cysteine, and aspartate.

A “ligand” is intended to include any substance that either inhibits or stimulates the activity of the membrane protein or that competes for the receptor in a binding assay. An “agonist” is defined as a ligand increasing the functional activity of a membrane protein (e.g. signal transduction through a receptor). An “antagonist” is defined as a ligand decreasing the functional activity of a membrane protein either by inhibiting the action of an agonist or by its own intrinsic activity. An “inverse agonist” (also termed “negative antagonist”) is defined as a ligand decreasing the basal functional activity of a membrane protein.

A “biological target molecule” is intended to include proteins such as e.g., membrane proteins, nucleic acids, carbohydrates, nucleoproteins, glycoproteins and glycolipids. In the present context the biological target molecule contains or has been manipulated to contain a metal ion binding site.

A “protein” is intended to include any protein, polypeptide or oligopeptide with a discernible biological activity in any unicellular or multicellular organism, including bacteria, fungi, plants, insects, animals or mammals, including humans. Thus, the protein may suitably be a drug target, i.e. any protein which activity is important for the development or amelioration of a disease state, or any protein which level of activity may be altered (i.e. up- or down-regulated) due to the influence of a biologically active substance such as a small organic chemical compound.

A “membrane protein” is intended to include but is not limited to any protein anchored in a cell membrane and mediating cellular signalling from the cell exterior to the cell interior. Important classes of membrane proteins include receptors such as tyrosine kinase receptors, G-protein coupled receptors, adhesion molecules, ligand- or voltage-gated ion channels, or enzymes. The term is intended to include membrane proteins whose function is not known, such as orphan receptors. In recent years, largely as part of the human genome project, large numbers of receptor-like proteins have been cloned and sequenced, but their function is as yet not known. The present invention may be of use in elucidating the function of the presumed receptor proteins by making it possible to develop methods of identifying ligand for orphan receptors based on compounds developed from metal ion chelates that bind to mutated orphan receptors into which artificial metal ion binding sites have been introduced.

“Signal transduction” is defined as the process by which extracellular information is communicated to a cell by a pathway initiated by binding of a ligand to a membrane protein, leading to a series of conformational changes resulting in a physiological change in the cell in the form of a cellular signal.

A “functional group” is intended to indicate any chemical entity which is a component part of the test compound and which is capable of interacting with an amino acid residue or a side chain of an amino acid residue of the membrane protein. A functional group is also intended to indicate any chemical entity which is a component part of the biological target molecule and which is capable of interacting with other parts of the biological target molecule or with a part of the test compound. Examples of such functional groups include, but are not limited to, ionic groups involved in ionic interactions such as e.g. the ammonium ion or carboxylate ion; hydrogen bond donor or acceptor groups such as amino, amide, carboxy, sulphonate, etc.; and hydrophobic groups involved in hydrophobic interactions, pi-stacking and the like.

A “wild-type” membrane protein is understood to be a membrane protein in its native, non-mutated form, in his case not comprising an introduced metal ion binding site

The term “in the vicinity of” is intended to include an amino acid residue located in the area defining the binding site of the metal ion chelate and at such a distance from the metal ion binding amino acid residue that it is possible, by attaching suitable functional groups to the test compound, to generate an interaction between said functional group or groups and said amino acid residue.

### IDENTIFICATION OR ENGINEERING OF METAL-ION BINDING SITES IN BIOLOGICAL TARGET MOLECULES TO BE EXPLOITED AS ANCHOR POINTS FOR LEAD COMPOUNDS

Nature of the Biological Target Molecules

The biological target molecules include but are not restricted to proteins, nucleoproteins, glycoproteins, nucleic acids, carbohydrates, and glycolipids. In the present context the biological target molecule contains or has been manipulated to contain a metal ion binding site. In preferred embodiments the biological target molecule is a protein, which may be for example a membrane receptor, a protein involved in signal transduction, a scaffolding protein, a nuclear receptor, a steroid receptor, a transcription factor, an enzyme, and an allosteric regulator protein, or it may be a growth factor, a hormone, a neuropeptide or an immunoglobulin.

In particularly preferred embodiments the biological target molecule is a membrane protein which suitably is an integral membrane protein, which is to say a membrane protein anchored in the cell membrane. The membrane protein is preferably of a type comprising at least one transmembrane domain. Interesting membrane proteins for the present purpose are mainly found in classes comprising 1-14 transmembrane domains.

ITM—membrane proteins of interest comprising one transmembrane domain include but are not restricted to receptors such as tyrosine kinase receptors, e.g. a growth factor receptor such as the growth hormone, insulin, epidermal growth factor, transforming growth factor, erythropoietin, colony-stimulating factor, platelet-derived growth factor receptor or nerve growth factor receptor (TrkA or TrkB).

2TM—membrane proteins of interest comprising two transmembrane domains include but are not restricted to, e.g., purinergic ion channels.

3, 4, 5TM—membrane proteins of interest comprising 3, 4 or 5 transmembrane domains includes but are not restricted to e.g. ligand-gated ion channels, such as nicotinic acetylcholine receptors, GABA receptors, or glutamate receptors (NMDA or AMPA).

6TM—membrane proteins of interest comprising 6 transmembrane domains include but are not restricted to e.g., voltage-gated ion channels, such as potassium, sodium, chloride or calcium channels.

7TM—membrane proteins of interest comprising i transmembrane domains include but are not restricted to G-protein coupled receptors, such as receptors for: acetylcholine, adenosine, norepinephrin and epinephrine, anaphylatoxin chemotactic factor, angiotensin, bombesin (neuromedin), bradykinin, calcitonin, calcitonin gene related peptide, conopressin, corticotropin releasing factor, amylin, adrenomedullin, calcium, cannabinoid, CC-chemokines, CXC-chemokines, cholecystokinin, conopressin, corticotropin-releasing factor, dopamine, eicosanoid, endothelin, fMLP, GABA_{B}, galanin, gastrin, gastric inhibitory peptide, glucagon, glucagon-like peptide I and II, glutamate, glycoprotein hormone (e.g. FSH, LSH, TSH, LH), gonadotropin releasing hormone, growth hormone releasing hormone, growth hormone releasing peptide (Ghrelin), histamine, 5-hydroxytryptamine, leukotriene, lysophospholipid, melanocortin, melanin concentrating hormone, melatonin, motilin, neuropeptide Y, neurotensin, nocioceptin, odor components, opiods, retinal, orexin, oxytocin, parathyroid hormone/parathyroid hormone-related peptide, pheromones, platelet-activating factor, prostanoids, secretin, somatostatin, tachykinin, thrombin and other proteases acting through 7TM receptor, thyrotropin-releasing hormone, pituitary adenylate activating peptide, vasopressin, vasoactive intestinal peptide and virally encoded receptors; in particular: adenosin, galanin. CC-chemokines, CXC-chemokines, melanocortin, bombesin, cannabinoid, lysophospholipid, fMLP, neuropeptide Y, tachykinin, dopamine, histamine, 5-hydroxytyptamine, histamine, mas-proto-oncogene, acetylcholine, oxytocin, human herpes virus encoded receptors, Epstein Barr virus induced receptors, cytomegalovirus encoded receptors and bradykinin receptors; preferably the galanin receptor type 1, leukotriene B4 receptor, CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, melanocortin-1 receptor, melanocortin-3 receptor, melanocortin-4 receptor, melanocortin-5 receptor, bombesin receptor subtype 3, cannabinoid receptor 1, cannabinoid receptor 2, EDG-2, EDG-4, FMLP-related receptor I, FMLP-related receptor II, NPY Y6 receptor, NPY Y5 receptor, NPY Y4 receptor, NK-1 receptor, NK-3 receptor, D2 receptor (short), D2 receptor (long), duffy antigen; US27, US28, UL33 and U78 from human cytomegalovius; U12 and U51 from human herpes virus 6 or 7, ORF74 from human herpes virus 8, and histamine H1 receptor, MAS proto-oncogene, muscarinic M1 receptor, muscarinic M2 receptor, muscarinic M3 receptor, muscarinic M5 receptor, oxytocin receptor, XCR1 receptor, EBI2 receptor, RDC1 receptor, GPR12 receptor or GPR3 receptor.

8, 9, 10, 11, 12, 13, 14TM—Membrane proteins of interest comprising 8 to 14 transmembrane domains include but are not restricted to e.g., transporter proteins, such as a GABA, monoamine or nucleoside transporter.

The membrane protein may also be a multidrug resistance protein, e.g. a P-glycoprotein, multidrug resistance associated prot, drug resistance associated protein, lung resistance related protein, breast cancer resistance protein, adenosine triphosphate-binding cassette protein, Bmr, QacA or EmrAB/TolC pump.

The membrane protein may also be a cell adhesion molecule, including but not restricted to for example NCAM, VCAM, ICAM or LFA-1.

Furthermore, the membrane protein may be an enzyme such as adenylyl cyclase.

In a particularly preferred embodiment of the invention, the biological target molecules are 7 transmembrane domain receptors (7TM receptors) also known as G-protein coupled receptors (GPCRs).

7TM overview—This family of receptors constitutes the largest super-family of proteins in the human body and a large number of current drugs are directed towards 7TM receptors, for example: antihistamines (for allergy and gastric ulcer), beta-blockers (for cardiovascular diseases), opioids (for pain), and angiotensin antagonists (for hypertension). These current drugs are directed against relatively few receptors, which have been known for many years. To date, several hundred 7TMs have been cloned and characterized, and tie total number of different types of 7TMs in humans is presumed to be between 1 and 2,000. The spectre of ligands acting through 7TMs includes a wide variety of chemical messengers such as ions (e.g. calcium ions), amino acids (glutamate, γ-amino butric acid), monoamines (serotonin, histamine, dopamine, adrenalin, noradrenalin, acetylcholine, cathecolamine, etc.), lipid messengers (prostaglandins, thromboxane, anandamide, etc.), purines (adenosine, ATP), neuropeptides (tachykinin, neuropeptide Y, enkephalins, cholecystokinin, vasoactive intestinal polypeptide, etc.), peptide hormones (angiotensin, bradykinin, glucagon, calcitonin, parathyroid hormone, etc.), chemokines (interleukin-8, RANTES, etc.), glycoprotein hormones (LH, FSH, TSH, choriogonadotropin, etc.) and proteases (thrombin). It is expected that a large number of the members of the 7TM superfamily of receptors will be suitable as drug targets. This notion is based on the fact that these receptors are involved in controlling major parts of the chemical transmission of signals between cells both in the endocrine and the paracrine system in the body as well as within the nervous system.

7TM receptor signalling—In 7TMs, binding of the chemical messenger to the receptor leads to the association of an intracellular G-protein, which in turn is linked to a secondary messenger pathway. The G-protein consists of free submits, an α-subunit that binds and hydrolyses GTP, and a βγ-subunit. When GDP is bound, the subunit associates with the βγ subunit to form an inactive heterotrimer that binds to the receptor. When the receptor is activated, a signal is transduced by a changed receptor conformation that activates the G-protein. This leads to the exchange of GDP for GTP on the α subunit, which subsequently dissociates from the receptor and the βγ dimer, and activates downstream second messenger systems (e.g. adenylyl cyclase). The a subunit will activate the effector system until its intrinsic GTPase activity hydrolyses the bound GTP to GDP, thereby inactivating the α subunit. The βγ subunit increases the affinity of the a subunit for GDP but may also be directly involved in intracellular signalling events.—7TM ligand-binding sites Mutational analysis of 7TMs has demonstrated that functionally similar but chemically very different types of ligands can apparently bind in several different ways and still lead to the same function. Thus monoamine agonists appear to bind in a pocket relatively deep between TM-III, TM-V and TM-VI, while peptide agonists mainly appear to bind to the exterior parts of the receptors and the extracellular ends of the TMs (Strader et al, (1991) J. Biol. Chem. 266: 5-8; Strader et al., (1994) Ann. Rev. Biochem. 63: 101-132; Schwartz et al. Curr. Pharmaceut. Design (1995), 1: 325-342). Moreover, ligands can be developed independent on the chemical nature of the endogenous ligand, for example non-peptide agonists or antagonists for peptide receptors, Such nonpeptide antagonists for peptide receptors often bind at different sites from the peptide agonists of the receptors. For instance, non-peptide antagonists may bind in the pocket between TM-III, TM-V, TM-VI and TM-VII corresponding to the site where agonists and antagonists for monoamine receptors bind. It has been found that in the substance P receptor, when the binding site for a non-peptide antagonist has been exchanged for a metal ion binding site through introduction of His residues, no effect on agonist binding was observed (Elling et al., (1995) Nature 374: 74-77; Elling et al. (1996) EMBO J. 15: 6213-6219). It is believed that the non-peptide antagonist and the zinc ions act as antagonists by selecting and stabilizing an inactive conformation of the receptor that prevents the binding and action of the agonist. This illustrates that drugs can be developed totally independent on knowledge of the endogenous ligand, since there need not be any overlap in their binding sites.

Generic numbering system for 7TMs—a useful tool in the identification and engineering of metal-ion sites is the generic numbering system for residue of 7TM receptors. The largest family of 7TM receptors is composed of the rhodopsin-like receptors, which are named after the light-sensing molecule from our eye. Within the many hundred members of the rhodopsin-like receptor family, a number of residues especially within each of the transmembrane segments are highly but not totally conserved. However, due to differences in the length of especially the N-terminal segment, residues located at corresponding positions in different 7TM receptors are numbered differently in different receptors. However, based on the conserved key residues in each TM, a generic numbering system has been suggested (J M Baldwin, EMBO J. 12(4), 1993, pp. 1693-1703; T W Schwartz, Curr. Opin Biotech. 5, 1994, pp. 434-444) In FIG. IV a schematic depiction of the structure of rhodopsin-like 7TMs is shown with one or two conserved, key residues highlighted in each TM: AsnI:18, AspII:10; CysIII:01 and ArgIII:26; TrpIV:10; ProV: 16; ProVI:15; ProVII:17. In relation to the present invention it is important that residues involved in for example metal ion binding sites can be described in this generic numbering system. For example, a tri-dentate metal ion site constructed in the tachyinin NK1 receptor (Elling et al., (1995) Nature 374, 74-77) and subsequently transferred to the kappa-opioid receptor (Thirstrup et al, (1996) J. Biol. Chem. 271, 7875-7878) and to the viral chemokine receptor ORF74 Rosenkilde et al., J. Biol. Chem. Jan. 8, 1999; 274(2), 956-61) can be described to be located between residues V:01, V:05, and VI:24 in all of these receptors although the specific numbering of the residues is very different in each of the receptors. It is only in the rhodopsin-like receptor family that a generic numbering system has been established; however, it should be noted that although the sequence identity between the different families of 7TM receptors is very low, it is believed that they may share a more-or-less common seven helical bundle structure. Thus, all the techniques described in the present invention can be applied to the other families of 7TM receptors with minor modifications. This generic numbering system together with general knowledge of the 3D structure of the 7TM receptors and knowledge from systematic metal-ion site engineering makes it possible to predict or identify the presence of metal-ion sites based on the DNA sequence coding for the 7TM receptor (see examples).

Orphan 7TM receptors—one embodiment of the invention is directed to a method of developing assay for orphan 7TM receptors by the introduction of metal-ion sites in the orphan receptor. During the cloning of 7TM receptors many “extra” receptors were discovered for which no ligand was known, the so-called orphan receptors. Today there are several hundreds of such orphan 7TM receptors. Based on characterization of their expression pattern in different tissues or expression during development or under particular physiological or patho-physiological conditions and based on the fact that the orphan receptors sequence-wise appear to belong to either established sub-families of 7TM receptors or together with other orphans in new families, it is believed that the majority of the orphan receptors are in fact important entities. As stated by representatives from the big pharmaceutical companies: Orphan 7TMs are “the next generation of drug targets” or “A neglected opportunity for pioneer drug discovery” (Wilson et al. Br. J. Pharmacol. (1998) 115: 1387-92; Stadel et al. Trends Pharmacol. Sci. (1997) 18: 430-37). Over the years ligands have been discovered for some of the orphan 7TM receptors, which then immediately have been recognized as “real” drug-targets, for example: nocioceptin (for pain) (Reinscheid et al. Science (1995) 270: 792-94), orexin (for appetite regulation and regulation of energy homeostasis) (Sakurai et al. Cell (1998) 92: 573-85), melanin-concentrating hormone (for appetite regulation) (Chambers et al. Nature (1999) 400: 261-65), and cysteinyl leukotrienes (inflammation, especially asthma) (Sarau et al. Mol. Pharmacol. (1999) 56: 657-63). In the latter case, a number of drugs (for example pranlukast, zafirlulast, montelukast, pobilukast) had in fact been developed in recent years against the receptor as a physiological entity without having access to the cloned receptor—which turned out to be a “well known” orphan receptor. The problem is that it is very difficult to characterize orphan receptors and find their endogenous ligands, since no assays are available for these receptors due to the lack of specific ligands—a “catch 22” situation. The present invention is aimed at eliminating this problem. By introducing metal ion binding sites in orphan receptors at locations where it is known from previous work on multiple other 7TM receptors with known ligands and with binding and functional assays that binding of metal ions and metal ion chelates will act as either agonists or more common as antagonists, then it will be possible to establish binding assays and functional assays for the orphan receptors. Binding of metal ion chelates can be monitored either through functional assays in cases where agonistic metal ion sites are created, or through ligand binding assays. For example, many aromatic metal ion chelators are by themselves fluorescent and can therefore directly be used as tracers in binding assays. Or, radioactive or other measurable indicators can be incorporated into the metal ion chelator. By establishing a metal ion chelator based receptor analysis for the orphan receptors, it will become possible to search for the elusive endogenous ligands or it will be possible to use the orphan receptors in various forms for drug discovery technology, for example high throughput screening. It should be noted that due to the initial lack of knowledge of the endogenous ligand and therefore also lack of knowledge of the binding site for this ligand in the 7TM receptor, there is a certain danger that the introduced metal ion binding site can interfere with ligand binding or signal transduction. However, based on metal ion site engineering in multiple 7TM receptors and on mutational mapping of binding sites in multiple 7TM receptors, it will be possible to introduce such metal ion sites at different locations in the receptor in an attempt to eliminate this problem. Moreover, an artificial binding site and binding analysis, which may interfere with the binding of the natural ligand, may still be useful for screening for receptor ligands, for example antagonists.

Source of the Biological Target Molecules

The biological target molecules of interest may be obtained in a useful form by different ways including but not limited to recombinantly, synthetically or commercially.

Cloning and expression—In a preferred embodiment the biological target molecule being a protein is obtained recombinantly. This can be achieved through cloning of the gene for the protein from genomic or cDNA libraries generally by the use of PCR techniques in accordance with standard techniques (eg. Sambrook et al. Molecular Cloning: A laboratory manual, 2. Ed Cold Spring Harbor Laboratory, New York 1989) and expression of the gene in a suitable cell. The nucleotide sequence encoding the target protein—and mutant versions thereof (see below)—may be inserted into a suitable expression vector for the purpose of expression and analysis in a host organism. Thus regulatory element ensuring either constitutive or inducible expression of the protein of interest should be present in the vector, including promoter elements. The host organism into which the nucleotide sequence is introduced may be any cell type or cell line, which is capable of producing the target molecule in a suitable form for the test to be performed including but not restricted to eg. yeast cells and higher eukarotic cells such as eg. insect or mammalian cells. Transformation of the cell line of choice may be performed by standard techniques routinely employed in the field as described eg. in Wigler et al. Cell (1978) 14: 725 and in accordance with standard techniques (Sambrook et al. Molecular Cloning A Laboratory Manual, 2. ed. Cold Spring Harbor Laboratory, Cold Spring Harbor, N.Y., 1989), In a particularly preferred embodiment the biological target molecule being a membrane protein is expressed and tested in mammalian cells usually within the membrane and usually in whole cells or in isolated membrane preparations, which is dealt with and described further in the examples presented in “EXPERIMENTAL”. Examples of suitable mammalian cell lines are the COS (ATCC CRL 1650. and 1651), BHK (ATCC CRL 1632, ATCC CCL 10), CHL (ATCC CCL39), CHO (ATCC CCL 61), HEK293 (ATCC CRL 1573) and NIH3T3 (ATCC CRL 1658) cell lines.

Isolation and purification—In the case where the biological target molecules is a soluble protein, for example an enzyme, a preferred source may be recombinantly produced protein, which subsequently is isolated and purified to a suitable purity and in a form suited for functional testing by various standard protein chemistry methods well known to those skilled in the art.

Functional testing of the biological target molecules

As part of the drug discovery process of the current invention, the biological target molecule comprising a natural or an engineered metal-ion binding site is contacted with a test compound for example consisting of a metal-ion in complex with a metal-ion chelator and any change in the biological activity of the biological target molecule is detected or the binding affinity of the test compound is determined.

Due to the diversity of biological target molecules, a wide variety of functional test can be performed depending on the individual target molecule and its functions. For example, for a soluble enzyme a suitable enzymatic analysis could be used on the purified enzyme (as described for Factor VIIa in the examples). For certain transcription factors a suitable gene-expression reporter assay could, for example be performed in a whole cell preparation. In a preferred embodiment of the invention the biological target molecule is a membrane protein and the effect of test compounds is monitored on the signal transduction process of the receptor, i.e. its ability to influence intracellular levels of for example cAMP, inositol phosphates, calcium mobilization etc. in response to the natural ligand (as described in “EXPERIMENTAL”). For instance, in the case of a 7TM receptor, this may entail the effect on signalling mediated trough the intracellular G-protein. In this way, the testing may reveal whether the binding of a metal-ion (complex) may affect the activity of the target in for instance an antagonistic or an agonistic fashion. For the most part tests are performed as dose-response analysis in which a range of concentration of metal-ion chelator complexes are exposed to the biological target molecule.

When appropriate, the binding affinity of the test compound to the biological target molecule is determined, for example in competition binding experiments against a suitable radioactively labelled ligand for the protein target (as described in “EXPERIMENTAL”). Or, the affinity of the test compound can in some cases be determined by use of a chelating agent which is in itself is detectable or which can be labelled with a detectable labelling agent.

Structure Testing of the Biological Target Molecules

In a preferred embodiment of the invention, the 3D structure of the test compound in complex with the biological target molecule is determined, for example by techniques such as X-ray analysis of crystals of the ligand-protein complex or, for example by nuclear magnetic resonance (NMR) spectroscopic analysis of complexes in solution—all known to those skilled in the art. In this way the amino acid residues located in the vicinity of the metal-ion site and the chemical interaction of the bifunctional test compound with specific residues in the biological target molecule can be determined as control and as basis for the structure-based design of further modifications of the lead test compound and design of new libraries of compounds. Further, the effect of the test compound on the structure of the biological target protein, domains of this and or effect on the interaction of the target protein with other proteins can be determined.

Identification of Metal-Ion Sites in Biological Target Molecules

In a preferred embodiment of the present invention, naturally occurring metal-ion sites are used as initial attachment sites for metal-ion chelating test compounds in the drug discovery process. In general, such natural metal-ion sites can be identified functionally by studying the effect of either free metal-ions or by the effect of a library of metal-ion chelator complexes on any function of the biological target molecule. Metal-ion sites can also be identified or confirmed by structural means as described above and location of the site can also be identified by careful, controlled mutagenesis, i.e. exchanging of the residues involved in metal-ion binding with residues not having this property. Natural metal-ion sites are interesting drug targets since binding of a drug at or close by a natural metal-ion site often will act as an allosteric agent, i.e. affecting the structure and function of the biological target molecule at a site different from the usual active site, where most ligands will bind and act (see below).

Natural metal-ion sites in proteins in general—Metal-ion sites are known to occur in many biological target molecules including but not restricted to, for example proteins, glycoproteins, RNA, etc. These sites can serve either structural or functional purposes. Some metal-ion sites are known to occur solely from functional data, for example Zn(II)-sites in ligand gated ion channels. Or previously unknown metal-ion sites are discovered in the crystal structure of the protein, as for example Zn(II) sites in rhodopsin. Independent on the physiological purpose of the naturally occurring metal-ion site they may be targeted by the technology of the present invention, where they are addressed not only by a metal-ion, but by a metal-ion in complex with a metal-ion chelator, which can affect the protein structure and function differently than the free metal-ion.

Natural metal-ion sites in 7TM receptors—naturally occurring metal ion sites have been described in two 7TM receptors: the tachykinin NK3 receptor (Rosenkilde et al. (1998) FEBS Lett. 439: 35-40) and the galanin receptor (Kask et al. (1996) EMBO J. 15: 236-240). In the NK3 receptor Zn(II) was shown to act as an enhancer (positive modulator) for agonist binding and action without itself being au agonist. Through mutagenesis the metal ion binding site was mapped to residues V:01 and V:05 at the extra-cellular end of TM-V. In the galanin receptor Zn(II) was shown to act as an antagonist for galanin binding, but the site was not characterized further (see “EXPERIMENTALS”). However, based on knowledge from metal-ion site engineering in 7TM receptors in general (see below) it is possible based on sequence analysis and molecular models to find previously unnoticed and often physiologically silent metal-ion sites in 7TM receptors. Some of these sites, for example the known one in the NK3 receptor, may be affected physiologically by free metal ions, for example when a receptor is expressed in brain regions where extra-cellular zinc concentrations may vary around 10⁻⁵ molar. However, many of the previously unnoticed metal ion sites may just be a reflection of the fact that polar, metal-ion binding ammo acid residues (for example: His, Cys, Asp etc.) frequently are found in the water-exposed main ligand-binding crevice of 7TM receptors. In one embodiment of the present invention, these residues are used as initial attachment sites for metal ion chelating test compounds, i.e. lead compounds in the drug discovery process (see for example the LTB4 receptor in “EXPERIMENTAL”).

Engineering of Metal-Ion Sites in Biological Target Molecules

It is generally known that metal-ion sites can be built into proteins by introduction of metal-ion chelating residues at appropriate sites. In a particularly preferred embodiment of the invention such sites are constructed at strategic sites in the biological target molecule with the purpose to serve as anchor sites for test compounds in a drug discovery process and thereby target the medicinal chemistry part of the process towards particularly interesting epitopes on the target molecule.

Mutagenesis—the nucleotide sequence encoding the target protein of interest may be subjected to site-directed mutagenesis in order to introduce the amino acid residue, which includes the metal-ion binding site. Site-directed mutagenesis may be performed according to well-known techniques. Eg. as described in Ho et al. Gene (1999) 77: 51-59. In a specific, non-limiting example the mutation is introduced into the coding sequence of the target molecule by the use of a set of overlapping oligonucleotide primer both of which encode the mutation of choice and through polymerisation using a high-fidelity DNA polymerase such as eg. Pfu Polymerase (Stratagene) according to manufacturers specifications. The presence of the site-directed mutation event is subsequently confirmed through DNA sequence analysis throughout the genetic segment generated by PCR. In order to generate a metal-ion binding site this may involve the introduction of one or more amino acid residues capable of binding metal-ions including but not restricted to, for example His, Asp, Cys or Glu residues.

Generally the mutated target molecule will initially be tested with respect to the ability to still constitute a functional, although altered, molecule through the use of an activity assay suitable in the specific case. It should be noted that although mutations in proteins may obviously occasionally alter the structure and affect the, function of the protein, this is by far always the case. For example, only a very small fraction (less than ten) of the many hundred Cys mutations performed in rhodopsin as the basis for site directed spin-labelling experiments and in for example the dopamine and other 7TM receptors as the basis for Cys accessibility scanning experiments have impaired the function of these molecules. Similarly, in the bacterial transport protein Lac-permease almost all residues have been mutated and only a few of these substitutions directly affect the function of the protein. Mutations will often also be performed in the biological target molecule to confirm or probe for the chemical interaction of test compounds with other residues in the vicinity of the natural or the engineered metal-ion site as an often integrated part of the general drug discovery method of the invention.

Metal-ion site engineering in protein targets in general—The method of the invention may suitably include a step of determining the location of, for example the metal ion binding amino acid residue(s) in a mutated protein and determining the location of at least one other amino acid residue in the vicinity of the metal ion binding amino acid residue, based on ester the actual three-dimensional structure of the specific biological target molecule in question (e.g. by conventional X-ray crystallographic or NMR methods) or based on molecular models based on the primary structure of the specific molecule together with the three-dimensional structure of the class of molecules to which the specific molecule belongs (e.g. established by sequence homology searches in DNA or amino acid sequence databases).

In the biological target molecule, the metal-ion binding site may suitably be introduced to serve as an anchoring, primary binding site for the test compound, which can thereby be targeted to affect a site in the biological target molecule having one or more of the following properties (the metal-ion site may be placed either within or close to this site):

a site where the biological target molecule binds to another biological target molecule, for example a regulatory protein.

a site which will control the activity of the biological target molecule in a positive or negative fashion (i.e. up-regulating or down regulating the activity of the biological target molecule), for example by an allosteric mechanism.

a site where the binding of the test compound will directly or indirectly interfere with the binding of the substrate or natural ligand or the binding of an allosteric modulatory factor for the biological target molecule.

a site where the binding of the test compound may interfere with the intramolecular interaction of domains within the biological target molecule, for example the interaction of a regulatory domain with a catalytic domain.

a site where binding of the test compound will interfere with the folding of the biological target molecule, for example the folding of a protein into its active conformation.

a site where the binding of the test compound will interfere with the cellular targeting of the biological target molecule.

a site where the binding of the test compound will stabilise a conformation of the biological target molecule, which presents an epitope normally involved in protein-protein interactions in a non-functional form.

This list of properties is by no means exhaustive and only serves to give some examples of the possibilities which can be obtained by targeting the test compound and thereby the final drug candidate to specific epitopes in the biological target molecule through the drug discovery process of the present invention.

This will potentially provide the ligand with other pharmacological properties than agents normally acting at the active site. It is for example likely that compounds binding at allosteric sites will be more efficacious in interfering with for example protein-protein interactions, which notoriously have been difficult as drug targets. Allosteric agents will, for example have the possibility of stabilising a conformation of the biological target molecule where major parts of the protein-protein interface are vastly different from the one enabling the normal interaction.

Metal-ion site engineering in 7TM proteins—in a preferred embodiment of the invention metalsites are introduced in 7TM receptors as part of the drug discovery process. Much experience has been obtained in building artificial metal-ion sites in 7TM receptors in general (Elling et al. Nature (1995) 374: 74-7; Elling et al. EMBO J. (1996)15:6213-9; Elling et al. Fold Des. (1997) 2: S76-SO; Elling et al., Proc Natl Acad Sci USA (1999) 96:12322-7; Sheikh et al. Nature. (1996) 383: 347-50). Based on this protein engineering work and on mutational analysis of ligand-binding sites as such at multiple locations in a number of wild-type 7TM receptors (Schwartz, T. W. (1994) Curr. Opin. Biotech 5: 434-444, Schwartz et al. Curr. Pharmaceut. Design (1995) 1: 325-342). However, in the present context such metal-ion sites are introduced in 7TM receptors as anchor points for lead compounds with the purpose of improving these compounds for high affinity binding and particular pharmacological profiles depending on their molecular interactions with the target molecule. The introduction of the sites is helped by molecular models of the 7TM receptors established on the basis of e.g., X-ray crystallographic data of a membrane protein of the same family, electron density maps of the membrane protein generated by cryo-electronmicroscopic analysis of two-dimensional membrane crystals (Baldwin, EMBO J. 12(4), 1993, pp. 1693-1703; Baldwin, Curr. Opinion. Cell. Biol. 6, 1997, pp. 180-190; Herzyk et al. J. Mol. Biol. 291(4), 1998, p. 741-754).

### SELECTION OF LEAD COMPOUND FROM LIBRARY OF METAL ION CHELATING COMPOUNDS

Test Compounds

Test compounds which have been found suitable for use in the present methods are any compound which is capable of forming a complex with a metal ion. All of the groups of a test compound which is attached directly to the metal atom or metal ion (central metal or coordinated metal)—whether ions or molecules—are the coordinating groups or ligands. A ligand attached directly through only one coordinating atom (or using only one coordination site on the metal) is called a monodentate ligand. A ligand that may be attached through more than one atom is multidentate, the number of actual coordinating sites being indicated by the terms bidentate, tridentate, tetradentate and so forth. Multidentate ligands attached to a central metal by more than one coordinating atom are called chelating ligands. A test compound for use in the present context is at least bidentate, i.e. it is a so-called metal ion chelator.

In the present context useful metal ion chelators generally have a log K value in a range of from about 3 to about 18 such as, e.g. from about 3 to about 15, from about 3 to about 12, from about 4 to about 10, from about 4 to about 8, from about 4.5 to about 7, from about 5 to about 6.5 such as from about 5.5 to about 6.5. K is an individual complex constant (also denoted equilibrium or stability constant). The constant's subscript 1, 2, 3 etc. indicates which coordination step the constant is valid for, i.e. K₁ is the complex constant for the coordination of the first ligand, K₂ is for the second ligand and so forth. log K can be determined as described in W. A. E. McBryde, “A Critical Review of Equilibrium Data for Protons and Metal Complexes of 1,10-Phenanthroline, 2,2′-bipyridyl and related Compounds.” Pergamon Press, Oxford, 1978.

In general, metal ion chelators can form complexes with different metal ions. In such cases it suffices for the purpose of the present invention that only one of the log K values for a given metal ion chelator is within the ranges specified above. Metal atoms or ions of particular relevance are: Co, Cu, Ni, Pt and Zn including he various oxidation steps such as, e.g., Co (II), Co (III), Cu (I), Cu (II), Ni (II), Ni (III), Pt (II), Pt (IV) and Zn (II).

More specifically, a test compound for use in a method according to the invention has at least two heteroatoms, similar or different, selected from the group consisting of nitrogen (N), oxygen (O), sulfur (S), selenium (Se) and phosphorous (P).

Test compounds which have been found to be useful in the present methods are typically compounds comprising a heteroalkyl, heteroalkenyl, heteralkynyl moiety or a heterocyclyl moiety for chelating the metal ion. The term “heteroalkyl” is understood to indicate a branched or straight-chain chemical entity of 1-15 carbon atoms containing at least one heteroatom. The term “heteroalkenyl” is intended to indicate a branched or straight-chain chemical entity of 2-15 carbon atoms containing at least one double bond and at least one heteroatom. The term “heteroalkynyl” is intended to indicate a branched or straight-chain chemical entity of 2-15 carbon atoms containing at least one triple bond and at least one heteroatom. The term “heterocyclyl” is intended to indicate a cyclic unsaturated (heteroalkenyl), aromatic (“heteroaryl”) or saturated (“heterocycloalkyl”) group comprising at least one heteroatom. Preferred “heterocyclyl” groups comprise 5- or 6-membered rings with 1-4 heteroatoms or fused 5- or 6-membered rings comprising 1-4 heteroatoms. The heteroatom is typically N, O, S, Se or P, normally N, O or S. The heteroatom is either an integrated part of the cyclic, branched or straight-chain chemical entity or it may be present as a substituent on the chemical entity such as, e.g., a thiophenol, phenol, hydroxyl, thiol, amine, carboxy, etc. Examples of heteroaryl groups are indolyl, dihydroindolyl, furanyl, benzofuranyl, pyridinyl, pyrimidinyl, quinolinyl, triazolyl, imidazolyl, thiazolyl, tetrazolyl and benzimidazolyl. The heterocycloalcyl group generally includes 3-20 carbon atoms, and 1-4 heteroatoms.

Particularly useful test compounds are those having at least two heteroatoms of general formula I

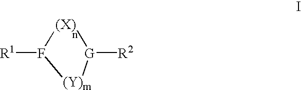

wherein F is N, O, S, Se or P, and G is N, O, S, Se or P;

at least one of (X)ₙ and (Y)ₘ is present and if n is O, then —(X)ₙ— is absent, and if m is 0, then —(Y)ₘ— is absent, and both n and m are not 0,

R¹ and R², which are the same or different, are radicals preferably selected from the group consisting of: hydrogen, a C₁-C₁₅ alkyl, C₂-C₁₅ alkenyl C₂-C₁₅ alkynyl, aryl, cycloalkyl, alkoxy, ester, —OCOR′, —COOR′, heteroalkyl, heteroalkenyl, heteroalkynyl, heterocycloalkyl, heterocycloalkenyl, heterocycloalkynyl or heteroaryl group, an amine, imine, nitro, cyano, hydroxyl, alkoxy, ketone, aldelhyde, carboxylic acid, thiol, amide, sulfonate, sulfonic acid, sulfonamide, phosphonate, phosphonic acid group or a combination thereof, optionally substituted with one or more substituents selected from the same group as R¹ and/or a halogen such as P, Cl, Br or I;

R′ is hydrogen, alkyl, substituted alkyl, alkenyl substituted alkenyl, alkynyl, substituted alkynyl, aryl, substituted aryl, arylalyl, substituted arylallyl, heteroalkyl, substituted heteroalkyl, heteroalkenyl, substituted heteroalkenyl, heteroalkynyl, heteroaryl, substituted heteroaryl, cycloalkyl, substituted cycloalkyl, cycloalkenyl, substituted cycloalkenyl, heterocycloallyl, substituted heterocycloalkyl, heterocycloalkenyl or substituted heterocycloalkenyl;

R¹ and/or R² optionally forming a fused ring together with any of F, (X)ₙ or a part of (X)ₙ G, (Y)ₘ or a part of (Y)ₘ or R¹ and R² themselves forming a fused ring;

X and Y are the same or different and have the same meaning as R′ such as, e.g., —CH₂—, CH₂—CH₂—, —CH₂—S—CH₂—, —CH₂—N—CH₂′, —CH═CH—CH═CH—, —(CH₂)_{d}—(Z)ₑ—(V)_{f}—(W)_{g}—(CH₂)ₕ—, —CH₂—O—CH₂—, wherein

each of Z and W are independently C, S, O, N, Se or P and

V is —CH— or —CH₂—;

(X)ₙ and/or (Y)ₘ optionally being substituted with one or more substituents selected from the same group as R¹ and/or a halogen such as F, Cl, Br or I;

n is 0 or an integer of 1-5,

m is 0 or a integer of 1-5,

e and/or g are an integer of 1-3,

d, f and/or h are an integer of 1-7.

As mentioned above m and n are not 0 at the same time. When m=0, the formula I is

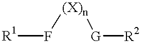

In the present context, the term “allyl” is intended to indicate a branched or straight-chain, saturated chemical group containing 1-15 such as, e.g. 1-10, preferably 1-8, in particular 1-6 carbon atoms, such as methyl, ethyl, propyl, isopropyl, butyl, sec. butyl, tert, butyl, pentyl, isopentyl, hexyl, isohexyl, heptyl etc.

The term “alkenyl” is intended to indicate an unsaturated alkyl group having one or more double bonds between two adjacent carbon atoms.

The term “alkynyl” is intended to indicate an unsaturated alkyl group having one or more triple bonds between two adjacent carbon atoms.

The term “cycloalkyl” is intended to denote a cyclic, saturated alkyl group of 3-7 carbon atoms.

The term “cycloalkenyl” is intended to denote a cyclic, unsaturated alkyl group of 3-7 carbon atoms having one or more double bonds between two adjacent carbon atoms.

The term “aryl” is intended to denote an aromatic (unsaturated), typically 5- or 6-membered, ring, which may be a single ring (e.g. phenyl) or fused with other 5- or 6-membered rings (e.g. naphthyl or anthracyl).

The term “alkoxy” is intended to indicate the group alkyl-O—.

The term “amino” is intended to indicate the group —NR′R″ where R′ and R″, which are the same or different, have the same meaning as R′ in Formula I. In a primary amine group, both R′ and R″ are hydrogen, whereas in a secondary amino group, either but not both R′ and R″ is hydrogen. R′ and R″ may also be fused to form a ring.

The term “ester” is intended to indicate the group COO—R′, where R′ is as indicated above except hydrogen, —OCOR″, or a sulfonic acid ester or a phosphonic acid ester.

Examples of halogen include fluorine, chlorine, bromine and iodine.

In the formula I above it is contemplated that if the valency of the heteroatoms F and/or G is more than 2 then further R¹ and/or R² groups are present adjacent to the F and/or C groups.

For the purpose of the present invention, other particular useful test compounds are those having the general formula II below

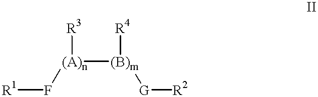

In the above formula II F, G, R¹ and R² have the same meaning as above. R³ and R⁴ have the same meaning as R¹ and/or R², and A and B have independently the same meaning as X and Y in formula I. n and m have the same meaning as in formula I except that n and m may be 0 at the same time and then the basic structure is R¹—F—G—R² and when n or m are O, respectively, then the basic structures of formula II are

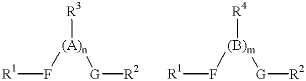

In formulas II (A) and (B) above the radicals R³ and R⁴ may be situated anywhere on A and B, respectively, or anywhere on (A)ₙ and (B)ₘ, respectively. For repeating units of e.g. A (or B) the group R³ (or R⁴) may be independently chosen in each of the repeating units.

Examples of interesting structures contained in test compounds for use in methods according to the present invention are given below.

The following formulas are based on the formula II above and F and/or G are nitrogen (N) or oxygen (O). T and Q are heteroatoms, and q and s independently are 0 or an integer of from 1 to 4. The meanings of q and s for q and/or s being 0 are he same as in Formula II for n and m. As an example, if q is 0 in Formula IIIA then the heterocyclic ring containing N is present, but the ring system does not contain any T. A circle indicates a fused alkyl, alkenyl, aryl, heteroalkyl, heteroalkenyl, heteroalkynyl or heteroaryl ring having from 3-7 atoms in the ring. R⁵ has the same meaning as R¹ and/or R². In Formulas III C-G, IV C and V C-D, T and/or Q may be placed anywhere in the cyclic system. This means for example that when q is 1, then one heteroatom T is present in the ring system and the position of the heteroatom is in principle freely chosen (of course the heteroatom F is also present, i.e. a total of two heteroatoms in the ring, when q is 1).

In the formulas below, the structure of the compounds are given in different structure levels. Firstly, a in very general form and then in more and more specific forms. Furthermore, an Formulas III are based on the same F-G-structure. The same applies for Formulas IV, V, VI and VII, respectively.

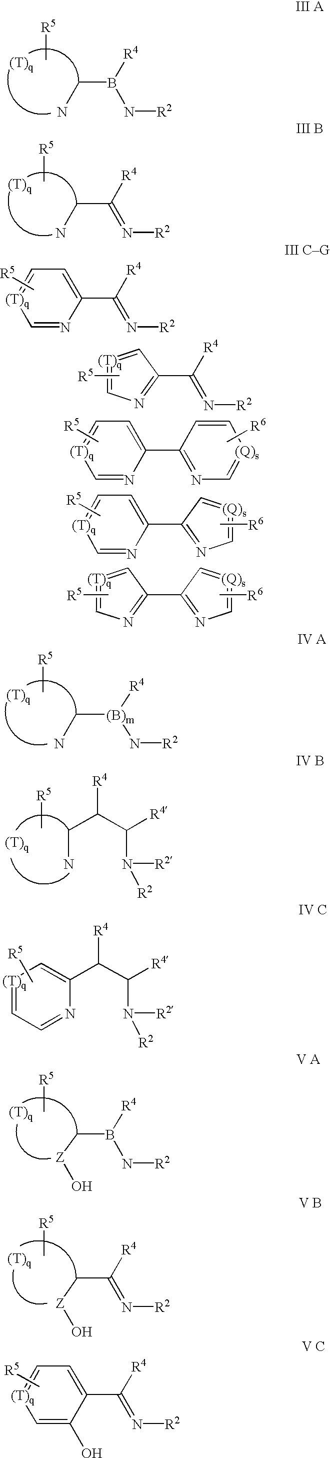

For the purpose of the present invention test compounds having a structure based on Formula III are suitable for use. Such compounds may comprise a heterocyclic moiety of the general formula VIII.

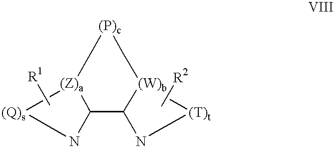

wherein R³, R⁴, Z, W and P are as defined herein before, a and/or b are an integer of 1-7 and c is 0 or an integer of 1-7, and each of Q and T is independently —CH— or —CH₂—, s is an integer of 1-7, and t is an integer of 1-7, are believed to be particularly suitable. When c is 0 in the above Formula VIII then —(P)_{c}— is absent, i.e. there is no bond between (Z)ₐ and (W)_{b}.

Test compounds in which the heterocyclyl moiety has the general formula IX.

[chemical structure]

wherein R³, R⁴, P, X and n are as indicated above, and r is 0 or an integer of 1-3, are also believed to be useful for the use in the present invention. When r is 0 then —Pᵣ— is absent.

Other suitable test compounds are those in which the structure corresponds to Formula VII. More specifically, the heterocyclyl moiety may have the general formula X

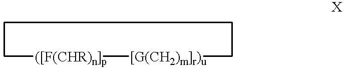

wherein F is N, O or S and G is N, O or S,

n is an integer from 1 to 5,

m is 0 or a integer from 1 to 5,

p and/or r are 0 or an integer from 1 to 8,

u is a integer from 1 to 8, and

R has the same meaning as R¹ in Formula I.

As an example of the meaning of p, r and/or u in the above formula the following applies: When r is 0 in the above Formula X then the Formula is

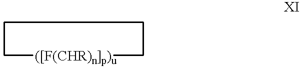

In analogy, the same meaning applies for p equal to 0, respectively, i.e. when p is 0 then the Formula is

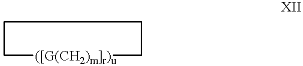

In all of the formulas given herein it is contemplated that when the valency of the heteroatoms F and/or G is more than 2 then, whenever relevant, further R¹ and/or R² groups are present adjacent to the F and/or G atoms.

Further useful test compounds are those in which the heterocyclic moiety is selected from a compound of formula XIIIa, XIIIb or XIIIc.

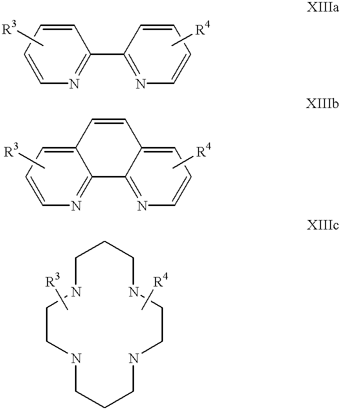

wherein R³ and R⁴ are as indicated above in formula I.

In Formulas VII, VIII, IX, X and XI the groups R³ and R⁴ only indicate that the ring(s) may be substituted with a group similar to R³ and/or R⁴. R¹ and R² in the meaning of formula I are included in the structures given above. Furthermore, it is understood that more than one R or substituent may be present whenever relevant and any R may also be substituted, cf. the meaning of eg. R¹ given under Formula I.

Examples of test compounds may be those in which the heterocyclic moiety is selected from a compound shown in Table 1:

TABLE I

| | |
|---|---|
| 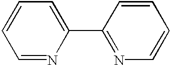 | 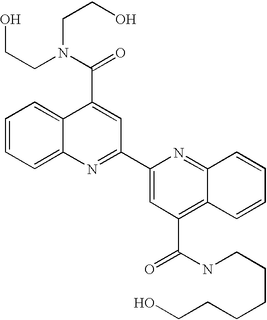 |
| 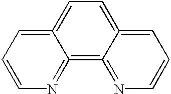 | 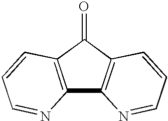 |
| 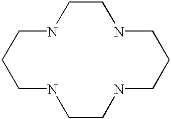 | 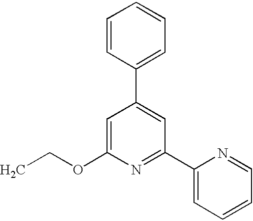 |
| 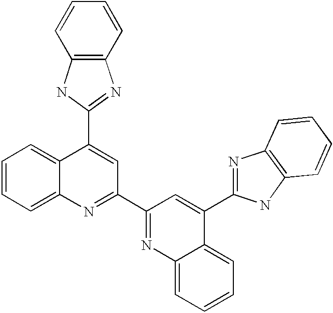 | 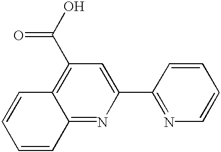 |
| 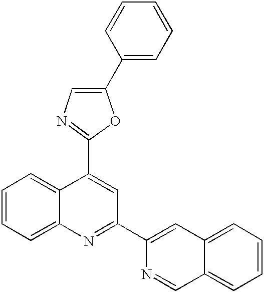 | 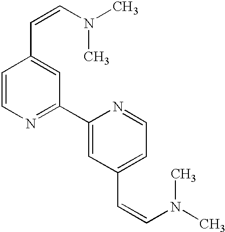 |

In the following Table II is given further examples of useful test compounds. The number given refers to an internal numbering system applied in the experiments performed.

TABLE II

| | |
|---|---|
| 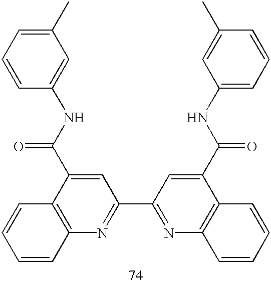 | 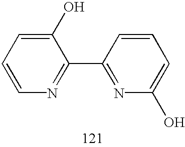 |
| 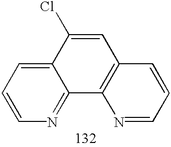 | 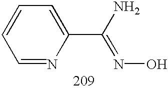 |
| 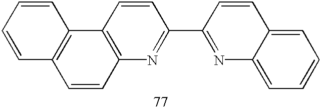 | 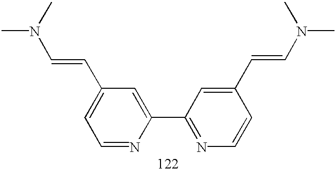 |
| 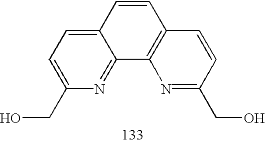 | 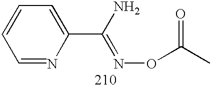 |
| 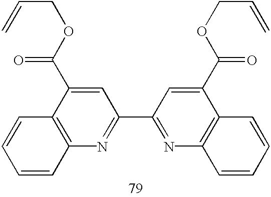 | 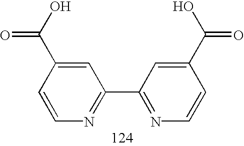 |
| 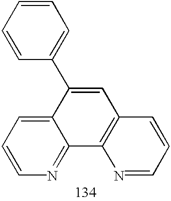 | 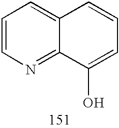 |
| 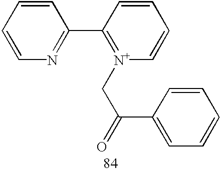 | 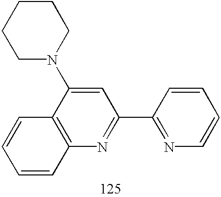 |
| 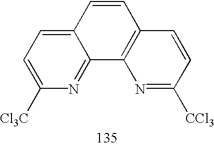 | 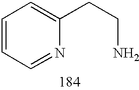 |
| 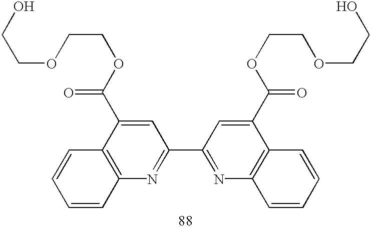 | 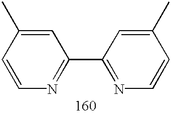 |
| 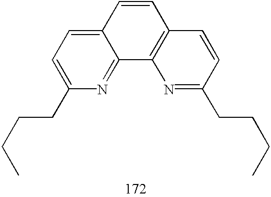 | 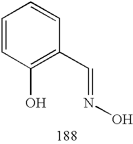 |
| 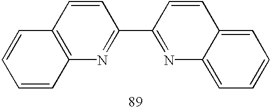 | 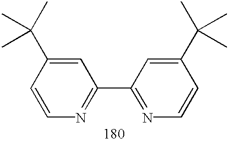 |
| 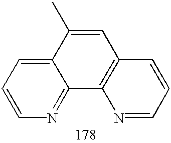 | 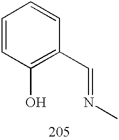 |
| 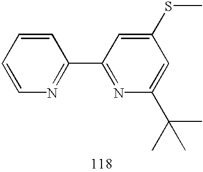 | 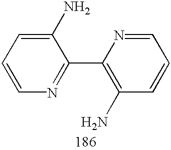 |
| 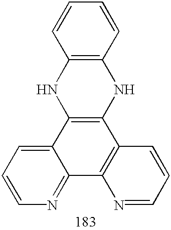 | 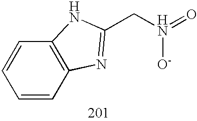 |
| 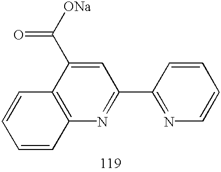 | 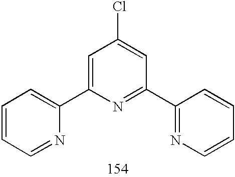 |
| 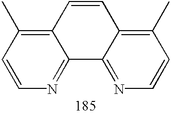 | 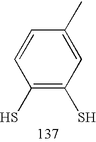 |
| 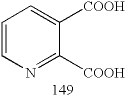 | 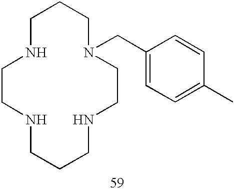 |
| 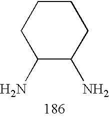 | 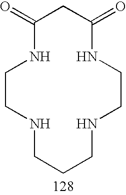 |

Metal atoms or ions forming the complex with the heteroalkyl or heterocyclyl moiety in the test compounds may advantageously be selected from metal atoms or ions which have been tested for or are used for pharmaceutical purposes.

Such metal atoms or ions belongs to the groups denoted light metals, transition metals, posttansition metals or semi-metals (according to the periodic system).

Thus the metal ion is selected from the group consisting of aluminium, antimony, arsenic, astatine, barium, beryllium, bismuth, boron, cadmium, calcium, cerium, cesium, chromium, cobalt, copper, dysprosium, erbium, europium, gadolinium, gallium, geranium, gold, hafnium, holmium, indium, iridium, iron, lanthanum, lead, lutetium, magnesium, manganese, mercury, molybdenum, neodymium, nickel, niobium, osmium, palladium, platinum, polonium, praseodymium, promethium, rhenium, rhodium, rubidium, ruthenium, samarium, scandium, selenium, silicon, silver, strontium, tantalum, technetium, tellurium terbium, thallium, thorium, thulium, tin, titanium, tungsten, vanadium, ytterbium, yttrium, zinc, zirconium, and oxidation states and isotopes thereof; in particular aluminium, antimony, barium, bismuth, calcium, chromium, cobalt, copper, europium, gadolinium, gallium, germanium, gold, indium, iron, lutetium, manganese, magnesium, nickel, osmium, palladium, platinum, rhenium, rhodium, rubidium, ruthenium, samarium, silver, strontium, technetium, terbium, thallium, thorium, tin, yttrium, zinc, and oxidation states or isotopes thereof; in particular cobalt, copper, nickel, platinum, ruthenium, and zink, and oxidation states and isotopes thereof, preferably calcium (II), cobalt (II) and (III), copper (I) and (II), europium (III), iron (II) and (III), magnesium (II), manganese (II), nickel (II) and (III), palladium (II), platinum (II) and (V), ruthenium (II), (III), (IV), (VI) and (VIII), samarium (III), terbium (III), zinc (II), or isotopes thereof, preferably cobalt (II) and (III), copper (I) and (II), nickel (II) and (III), zink (II) and platinum (II) and (V), or isotopes thereof.

For the present purpose, a particularly favourable test compound is a chelate between any of the test compounds of the formulas mentioned above and any of the metal atoms or ions mentioned above. In particular chelates between any of the test compounds and any of atom or ion of Co, Cu, Ni, Zn, Rn and Pt are of interest in methods of the present invention. Especially, chelates like e.g. metal ion-phenanthroline complex, metal ion-bipyridyl complex and metal ion-1,4,8,11-tetraazacyclotetradecane complex are suitable for use in methods of the present invention such as, e.g., a Cu²⁺-phenanthroline complex, a Zn²⁺-phenanthroline complex, a Cu²⁺-bipyridyl complex, a Zn²⁺-bipyridyl complex, a Ca²⁺-bipyridyl complex, a Cu²⁺-1,4,8,11-tetraazacyclotetadecane, a Zn²⁺-1,4,8,11-tetraazacyclotadecane.

Libraries

The invention also relates to chemical libraries of test compounds and their use in drug discovery processes. More specifically, a chemical libery is claimed comprising test compounds according to the above-mentioned formula I and wherein the test compound is or is not in chelated form with any of the metal ions mentioned above. A chemical library of salt, solvates or complexes of the above-mentioned metal ions is also claimed. Besides the chemical structure, the test compounds contained in the libraries must fulfil certain criteria with respect to molecular weight (at the most 2000 such as, e.g., at the most 1500, at the most 1000, at the most 750, at the most 500), lipophilicity (log P at the most 7 such as, e.g., at the most 6 or at the most 5), number of hydrogen bond donors (at the most 15 such as, e.g. at the most 13, 12, 11, 10, 8, 7, 6 or at the most 5) and number of hydrogen bond acceptors (at the most 15 such as, e g, at the most 13, 12, 11, 10, 9, 7, 6 or of the most 5).

Libraries of test compounds or of salt, solvates or complexes of the above-mentioned metal ions which find use herein will generally comprise at least 2 compounds, often at least about 25 different compounds such as, e.g., at least about 100 different compounds, at least about 500 different compounds, at least about 1000 different compounds or at least about 1000 different compounds. The method by which the population of compounds are prepared are not critical to the invention and a person skilled in the field of chemistry will be able to select suitable synthetic methods for the preparation of the compounds.

### CHEMICAL OPTIMIZATION OF LEAD COMPOUND FOR SECONDARY INTERACTION POINTS IN THE BIOLOGICAL TARGET MOLECULE

Identification of Chemical Interactions

The chemical optimization of the test compound can be guided by detailed knowledge of the 3D structure(s) of the biological target molecule, preferentially determined in complex initially with the un-substituted metal-ion chelator and subsequently in complex with the chemically modified metal-ion chelator in which attempts have been made to establish first one secondary interaction and subsequently further secondary or tertiary interactions. For some biological target molecules such as soluble proteins this can be achieved through for example crystallization and standard X-ray analysis procedures or through, for example NMR analysis of the complex in solution again using standard procedures.

For membrane proteins high resolution structures are in general not available. However determination of chemical interactions may be performed using a generic three-dimensional model of the membrane protein showing the spatial arrangement of the amino acid residues defining the area of the metal ion binding site. Such a determination is then performed using site-directed mutagenesis of a least one amino acid residue potentially involved in interaction with said functional group of the test compound other than he metal ion. Followed by expression of the mutated membrane protein in a suitable cell, contacting said cell or a portion thereof including the mutated membrane protein with the test compound, and determining any effect on binding in a competitive binding assay using a labelled ligand of the membrane protein, detection of any changes in signal transduction from the membrane protein or using a chelating agent which is in itself detectable or labelled with a detectable labelling agent. If an amino acid residue involved in interaction with such a functional group of the test compound is mutated to one, which is not this may be detected as a decrease in binding or other activity

Generation of New Specific Interactions

During the chemical optimisation of the test compound methods developed for structure-based drug discovery in general can be utilized, as knowledge of the 3D structure of the target epitopes makes it possible to apply classical structure-based approaches such as structure-based library design for the establishment of secondary and tertiary interaction sites for the lead compound in the target molecule. However, it should be noted, that a major advantage and difference of the present method is, that the lead compound is anchored to a particular site and thereby to a certain degree in a particular conformation in the biological target molecule through binding to the bridging metal-ion site while the compound is being optimized for chemical recognition with the target molecule.

In the case of membrane proteins suitable X-ray structures are generally not available. However, the molecular models are often rather detailed and in the case of the 7TM receptors they are in fact rather precise and correspond well with the X-ray structure of rhodopsin which was recently published. Thus the combination of relatively good molecular models (which have allowed for the construction of interhelical metal-ion sites) and the present method does to a certain degree compensate for the lack of detailed knowledge of the 3D structure of the target molecule because the lead compound is anchored and thereby create a fix-point for the subsequent medicinal chemical optimization point guided by the molecular models.

By using relatively flexible spaces in between the metal-ion chelating moiety and the variable chemical moiety of the test compound it becomes possible to probe for interaction or binding to structurally and functionally interesting epitopes of the biological target molecule with chemical moieties, which due to their intrinsic low affinity would normally not be detectable in the analytical systems on their own. Due to the local high concentration of the chemical moieties, which is created by the tethering to the metal-ion chelating moiety bound to the metal-ion site, these compounds can now be detected.

Use of Test Compounds in In Vivo Target Validation

In an embodiment of the invention the method will be used to increase the affinity and specificity of metal-ion chelator compounds to be used in pharmacological knock-out applications for in vivo target validation; i.e. to determine the effect of a specific agonist or antagonist for a biological target molecule. Here, the compounds will be used as metal-ion chelator complexes. This procedure has in principle been described previously (Elling et al. (1999) Proc. Natl. Acad. Sci. USA 96:12322-12327); however only for basic metal-ion chelating agents. The technology is based on the introduction of a silent metal-ion site in a potential drug target, i.e. creation of a metal-ion site in which the mutations do not affect the binding and action of the endogeneous ligand for the receptor. When such a metal-ion site engineered receptor is introduced into an animal by classical gene-replacement technology, i.e. exchange of the endogenous receptor with the metal-ion site engineered receptor, then the animals will develop normally without any compensatory mechanisms, which otherwise frequently impair the interpretations of the phenotypes of the animals in classical gene knock-out technology. In the adult animals or whenever it is found appropriate the animals are then treated with an appropriate metal-ion-chelating agent which then will act as an antagonist (or agonist) and turn off (or on) the function of the metal-ion site engineered receptor. Currently, this approach is impaired by the fact, that the generally available metal-ion chelating agents only will bind with at best ?M affinity to the metal-ion site engineered biological target molecule, which will give similar ?M or lower antagonistic potencies. These relatively low potencies and the relative low specificity of the basic test compounds impairs the general applicability of the technology due to simple pharmacokinetic and toxicology problems. With the technology presented in the present invention above it will be possible to increase the affinity of metal-ion chelators significantly, which will make it considerably more easy to reach therapeutic, efficient antagonistic concentrations of the metal-ion chelator in the animals and also to increase the “therapeutic window” due to the higher degree of selectivity of the compounds caused by the establishment of more than one molecular interaction point. Establishment of just a single suitable charge-charge interaction will increase the affinity of the metal-ion chelator by 10 to 100-fold or more.

### CHEMICAL OPTIMIZATION OF LEAD COMPOUND FOR HIGH AFFINITY INTERACTION WITH WILD TYPE BIOLOGICAL TARGET MOLECULE

Exchange of Metal Ion Anchor with “Ordinary” Chemical Interaction

In the case, where the initial binding of the metal-ion chelator was obtained through mutational introduction of an anchoring metal-ion site in the biological target molecule, a final step of optimization will have to be performed to obtain high affinity binding or potency on the wild-type target molecule without the metal-ion bridge. Through the methods described in the previous experiments, the metal-ion chelator lead compound will gradually be optimized for interactions with chemical groups in the biological target molecule spatially surrounding the metal-ion site—i.e. interactions with chemical groups found also in the wild-type target molecule. Thus, the test compound will gradually increase its affinity not only for the metal-ion site engineered molecule but also for the wild-type biological target molecule. When two to three secondary interaction points have been established, the affinity of the test compound for the wild-type target molecule, which is being tested in parallel with the metal-ion site engineered molecule, will have reached micro-molar affinities, i.e. a lead compound on the wild-type target molecule has been created. At this point one or more of the following three approaches will be followed: 1) structure-based further chemical optimization of the compound in general aiming at improving recognition at various known chemical moieties of the target molecule; 2) structure based further chemical optimization of the compound at which the “metal-ion site bridge” is exchanged by a more classical type of chemical interaction with the residue(s) which had been modified to create the metal-ion site in the biological target molecule. Here advantage can be taken of the fact that the geometry of the metal-ion site anchor is well known in general and, that relatively limited structure-based libraries can be established to create a new type of interaction; 3) further chemical optimization of the compound through more-or-less random generation of chemical diversity in general in the compound.

Applications

The small organic molecular ligands (compounds) identified according to the methods of the present invention will find use as e.g. drug compounds with abortifacient, acromegalic, alcohol deterrent, amebicidic, anabolic, analeptic, analgesic, anesthetic, antiacne, antiallergic, ophthalmic, anti-Alzheimer's disease, antianginal, antiarrhythmic, antiarthritic, antiasthmatic, antibacterial, antibiotic, anticancer, anticholelithogenic, anticoagulant, anticonvulsant, antidepressant, antidiabetic, antidiarrheal, antiemetic, antiepileptic, antiestogen, antifungal, antiglaucoma, antihistamine, antihypertensive, antiinflammatory, antilipidemic, antimalarial, anitimigraine, antinauseant, antineoplastic, antiobesity, antiparasitic, antiparkinsonian, antiperistaltic, antiprogestogen, antiprolactin, antiprostatic hypertrophy, antipsoriatic, antipsychotic, antirheumatic, antisecretory, antiseptic, antispasmodic, antithrombotic, antitussive; antiulcer, antiviral, anxiolytic, bronchodilator, calcium regulator, cardioprotective, cardiostimulant, cardiotonic, cephalosporin, cerebral vasodilator, chelator, choleretic chrysotherapeutic, cognition enhancer, congestive heart failure, coronary vasodilator, cystic fibrosis, cytoprotective, dependence treatment, diuretic, dyslipidemia, enzyme, expectorant, fertility enhancer, fibrinolytic, gastoprokinetic, Gaucher's disease, growth hormone, growth hormone insensitivity, haemophilia, heart failure, hematologic, hematopoetic, hemostatic, hepatroprotective, hormone, hypecphenylalaninemia, hyperprolactinemia, hypertensive, hypnotic, hypoammonnuric, hypocalciurc, hypocholesterolemic, hypoglycemia, hypolipaemic, hypolipidemic, idiopathic hypersomnia, immunomodulator, immunostiulant, immunosuppressant, beta-lactamase inhibitor, leukopenia, lung surfactant, mucolytic, muscle relaxant, multiple sclerosis, muscle relaxant, narcotic antagonist, nasal decongestant, neuroleptic, neuromuscular blocker, neuroprotective blocker, neuroprotective, nootropic, non-steroid antiinflammatory disease disease (NSAID), osteoporosis, Paget's disease, platelet aggregation inhibitor, platelet antiaggregant, pneumonia, precocious puberty progestogen, protease inhibitor, psychostimulant, 5-alpha-reductase inhibitor, respiratory surfactant, subarachnoid hemorrhage, thrombolytic, ulcerative colitis, urolithiasis, urologic, vasoprotective, vulnerary and wound healing properties. Important proteins for the present purpose are proteins, which may be stabilised in an active or inactive conformation by a biologically active substance. In this way, it may be possible to obtain an effect of a test compound of the type described herein irrespective of whether the active site of the protein is known, or whether the structure of the active site has been resolved (e.g. by X-ray crystallisation). Examples of such proteins are enzymes, receptors, hormones and other signalling molecules, transcriptional factors and regulators, intra- or extracellular structural proteins, in particular actins; adaptins; antibodies; ATPases; cyclins, dehydrogenases; GTP-binding proteins; GTP/GDP-exchange factors; GTPase activating proteins; GTP/GDP dissociation inhibitors; chaperones; histones; histone acetyltransferases & deacetyltransferases; hormones and other signalling proteins and peptides; kinases; lipases; major facilitator superfamily proteins; motorproteins; nucleases; polymerases; isomerases; proteases; protease inhibitors; phosphatases; ubiquitin-system proteins; membrane proteins including receptors, transporters and channels; transcription factors and tubulins; preferably membrane receptors; nuclear receptors, zinc finger proteins; proteases, tyrosine kinases and matrix proteins. Other important proteins for the present purpose are proteins whose biological activity is regulated by their cellular targeting and whose biological activity therefore can be modulated by drugs, which alter their cellular targeting with or without altering their actual intrinsic activity.

The invention is further illustrated in the following non-limiting examples.

### LEGEND TO FIGURES

FIG. I.1

Identification of naturally occurring metal-ion binding site in the 7TM leukotriene LTB4 receptor

Whole cell competition binding experiment with COS-7 cells expressing the wild type and mutant variants of the leukotriene LTB4 receptor using [³H]-LTB4 as the radioligand.

Panel A. Pity of Cu(II), 2,2′-bipyridine and the complex therof in the wild type LTB4 receptor.

Panel B. Affinity of Cu(biprydine) in mutant forms of the LTB4 receptor in which the metal-ion binding is severely imparired.

Panel C. Helical wheel diagram illustrating the transmembrane segments of the LTB4 receptor, The two cysteine residues within the a membrane segment III which have been identified as critical for metal-ion chelator complex binding, Cys93 and Cys97 are indicated in dark gray.

FIG. I.2

Identification of naturally occurring metal-ion binding site in the 7TM galanin receptor

Whole cell competition binding experiment with COS-7 cells expressing the wild type and mutant forms of the galanin receptor using [¹²⁵I]-galanin as radioligand.

Panel A. Affinity of the free copper metal-ion, the free chelator and the phenanthroline complex on the wild-type galanin receptor.

Panel B. Affinity of the copper-phenanthroline copmplex on two mutant forms of the galanin receptor, in which the binding of the metal-ion complex is impaired.

FIG. I.3

Identification of naturally occurring metal-ion binding site in the 12TM protein, the dopamine transporter.

Competition analysis of uptake of [³H]-dopamine in whole COS-7 cells expressing the dopamine transporter.

Panel A. Uptake of [³H]-dopamine by the wild-type dopamine transporter in the presence of free metal zinc-ion and zinc in complex with the chelator 2,2′-bipyridine.

Panel B. Dopamine uptake analysis in a mutant form of the dopamine transporter, [H193K], in which binding of the metal-ion complex has been eliminated (Noregaard et al. EMBO J. (1998) 17: 4266-4273).

Panel C. Effect of metal-ion complex formation on the ability to inhibit [³H]-dopamine uptake in the wild-type and [193K] mutant dopamine transporter. (or compounds, 209 and 210, see list of compounds in Appendix).

FIG. II.1

Binding of various metal-ion complexes to a library of inter-helical metal-ion sites engineered into the tachykinin NK1 receptor.

COS-7 cells expressing various engineered forms of the NK1 receptor were analyzed by competition binding using [¹²⁵I]-Substance P as radioligand

Panel A. IC₅₀ values for the zinc and copper metal-ions and complexes thereof with the chelators, 2,2′-bipyridine and phenanthroline are presented in the table. N indicated the number of experiments performed.

Panel B. Data obtained using the chelator cyclam are presented for the NK1 mutant in which an inter-helical metal-ion site has been generated through the introduction of the His V:05 ;His VI:24 exchanges.

Panel C. A helical diagram representing the four sets of inter-helical metal-ion sites which appear in Panel A are indicated.

FIG. II.2

Re-engineering of a metal-ion chelator binding site in the 12TM dopamine transporter.

Dopamine uptake was analysed in COS-7 cells expressing the wild type and mutant forms of the dopamine transporter in competition with the metal-ion chelator complex, zinc(II)-2,2′-bipyridine. The two panels show two forms of re-engineered dopamine transporters in which the ability to bind the metal-ion chelator complexes have been reconstituted following the elimination of the His193 interaction point.

FIG. III.1

Structure-activity-relationship of antagonist metal-ion complexes in the galanin and the leukotiene LTB4 receptors.

Panel A. Competition binding analysis in COS-7 cells expressing the galanin receptor. Binding of [¹²⁵I]-galanin was analysed in the presence of various copper-ion chelator complexes.

Panel B. Competition binding analysis in COS-7 cells expressing the LTB4 receptor. Binding of [³H]-LTB4 was analysed in the presence of various copper-ion chelator complexes.

For structures of the chelators employed in both panels, see Appendix.

FIG. III.2

Structure-activity relationship of antagonist metal-ion complexes in the metal-ion site engineered tachykinin NK1 receptor

Binding of [¹²⁵I]-Substance P was analysed in COS-7 cells expressing NK1 receptor which have been engineered to bind the zinc metal-ion. Ligand binding is presented in competiton with the zinc metal-ion, the zinc-1,10-phenanthroline complex and with other zinc-chelator complexes as indicated. For structures of the chelators, see Appendix.

FIG. III.3

Structure-activity relation ship of agonistic metal-ion complexes in the metal ion site Beta2-adrenergic receptor.

The effect of Cu(II) and copper-chelator complexes on stimulation of accumulation of intracellular cAMP was analyzed in COS-7 cells expressing the beta2-adrenoceptor.

Panel A. Washing experiment demonstrating the reversibility of the simulatory action of the metal-ion complexes.

Panel B. The effect of copper and complexes in the wild-type beta2-AR and in engineered forms of the receptor.

Panel C. Dosis-response analysis of selected copper-chelator complexes on the [F289C;N312C] beta2-AR.

FIG. III.4

Structure-activity relationship of antagonistic metal-ion complexes in a soluble protein, the enzyme FVIIa.

A comparison of selected meal-ion complexes on the binding of [3H]-LTB4 and the inhibition of the enzymatic activity of the active form of Factor VII (FVIIa) in COS-7 cells expressing respectively the LTB4 receptor (Panel B) and the FVIIa (Panles A and C). For stucture of the chelators see the Appendix.

FIG. III.5

Structure-based optimization of metal-ion chelators for secondary interactions in the CXCR4 receptor and other biological targets.

Helical wheel diagram for the CXCR4 receptor. The Asp 171 residue present in the transmembrane segment IV, and which is considered a major attachment site for the binding of the cyclam chelator is shown in white on black. Positions which in combination are proposed to constitute putative metal-ion binding sites are high-lighted in pairs and in black on dark gray.

FIG. IV

Schematic depiction of the structure of rhodopsin-like 7TMs with one or two conserved, key residues highlighted in each TM: AsnI:18; AspII:10; CysIII:01 and ArgIII:26; TrpIV:10; ProV:15; ProVII:17.

FIG. V

A table of test compounds wherein log K values are given.

### EXAMPLES

The examples presented encompass naturally occurring as well as specifically engineered metal-ion binding sites in a number of different proteins representing several different classes of membrane proteins: 7TM proteins (examples being various G-protein coupled receptors), and 12TM proteins (example—the dopamine transporter) as well as an example comprising a soluble protein, Factor VIIa, the active form of the FVII protease.

The examples are chosen with the intent of illustrating the sequential and rational process through which small organic compounds, the metal-ion chelators, may be identified as ligands and subsequently optimized with respect to the affinity by which they recognize the protein targets.

Overall, the examples serve to illustrate how the activity of potential drug targets may be affected through interaction with small metal-ion chelators and importantly how the present technology provides the opportunity to aim the active drug candidates towards functionally significant domains of the target. Throughout this section, ‘the affinity’ of the metal-ion chelator complexes refers to the ability of the complex to displace the binding of a radioligand and the potency of the metal-ion chelator complexes refers to the ability of the substances to activate or inactivate the drug targets.

I. Binding Of Metal-Ions and Metal-Ions Complexes To Various Drug Targets With Natural Metal-Ion Sites

The examples compiled in this section illustrate how metal-ion binding sites may be identified in the native forms of various drug targets, and how these sites may be addressed by metal-ions in complex with certain chelators, as observed either through an effect on the binding affinity of a radioactive ligand or through a direct effect on activation or inactivation of the target.

### Example I.1

### Identification of a Naturally Occurring Metal-ion Chelator Binding-site in the 7TM Leukotriene LTB4 Receptor

The present example illustrates how the presence of a previously unnoticed, naturally occurring metal-ion binding site within a transmembrane segment of a 7TM receptor may be predicted through analysis of the nucleotide sequence of the gene coding for the protein and how it can subsequently be experimentally identified. Briefly, molecular models of 7TM receptors can be built based on the deduced amino acid sequence and identification of the seven transmembrane segments (eg. Unger at al, (1997) Nature 389: 203-206). In these molecular models, illustrated in the helical wheel diagram shown in FIG. I.1B, potential metal-ion sites can be identified by the presence of metal-ion binding residues, for example histidine, cyoteine, or aspartate residues located in suitable relative positions, for example in an i and i+4 arrangement (i.e. with three residues in between) on a helical face within the so-called main ligand-binding crevice of the receptor between TM-II, III, IV, V, VI, and VII (Schwartz et al, (1996) Trends Pharmacol. Sci. 17: 213-216).

Methods—The leukotriene LTB4 receptor cDNA was cloned by PCR from a leukocyte cDNA library, built into an eukaryotic expression vector and introduced into COS-7 cells by a standard calcium phosphate transfection method. One day after transfection the cells were transferred and seeded in multi-well plates for assay. The number of cells plated per well was chosen so as to obtain 5 to 10% binding of the radioligand added. Two days after transfection the cells were assayed for the presence of [³H]-LTB4 binding activity. Radioligand was bound in a buffer composed of 50 mM Tris-HCl (pH 7.4), 3 mM MgCl₂, 0.1 % BSA, 100 mg/ml Bacitracin and displaced in a dose dependent manner by unlabelled LTB4 ligand. The assay was performed in duplicate for 3 hours at 4° C., and stopped by washing twice in buffer. Cell associated, receptor bound radioligand was determined by the addition of lysis buffer (48% urea, 2% NP-40 in 3M acetic acid). The concentration of radioligand in the assay corresponds to a final concentration of 45 pM. The metal-ion chelating complex, 2,2′-bipyridine was added in a two-fold molar excess in order to ensure that no free metal-ion was present.

Results and discussion—As shown in the helical wheel diagram of the leukotriene LTB4 receptor (FIG. I.1C), two Cys residues are located on the face of TM-III pointing inwards, i.e. towards the main ligand-binding pocket of the receptor (positions III:04, Cys⁹³ and III:08, Cys⁹⁷). Theoretically these residues could constitute a metal-ion binding site. The actual presence of a naturally occurring metal-ion binding site in the leukotriene LTB4 receptor is demonstrated by the fact, that binding of the radioligand, [³H]-LTB4 to the receptor expressed in COS-7 cells could be displaced by Cu(II), IC₅₀=70 μM (FIG. I.1A). In agreement with the fact that the proposed metal-ion site is located in the main ligand-binding pocket of the receptor, i.e. with amble space towards the center of the receptor, the complex between the metal-ion and the chelator, 2,2′-bipyridine bound equally well as the free metal-ion, i.e. the 2,2′-bipyridine did neither impair nor improve the binding; affinity (FIG. I.1A). As shown in FIG. I.1B, Ala-substitution of Cys⁹³ severely deduced amino acid sequence and identification of the seven transmembrane impaired the effect of the metal-ion chelator complex on LTB4 binding, Ala-substitution of Cys⁹⁷ also clearly impaired the effect of the metal-ion complex. The combined substitution of both Cys residues totally eliminated the metal-ion chelator effect (FIG. I.1.A) demonstrating that these two residues on the central face of TM-III are involved in the binding of the metal-ion chelator complex. Thus, the two residues represent a naturally occurring intra-helical ‘bis-Cys-site’, which can be addressed with for example Cu(II) in complex with bipyridime.

### Example I.2

### Identification of Naturally Occurring Metal-ion Chelator Binding Site in the 7TM Galanin Receptor

Whereas the naturally occurring metal-ion site in the LTB4 receptor is located within a transmembrane helix, the metal-ion site in the receptor for the neuropeptide galanin exemplifies the identification of an inter-helical metal ion site in a 7TM receptor. Furthermore, this is an example in which the metal-ion chelator positively contributes to the affinity of the metal-ion.

Methods—The galanin receptor cDNA was introduced into COS-7 cells by the standard calcium phosphate transfection method. The cells were transferred and seeded in multi-well plates for assay one day following the transfection and the number of cells plated per well was adjusted for each individual (wild type and mutant) construct aiming at the binding of 5 to 10% of the radioligand present in the assay. Two days post-transfection the cells were assayed for the presence of [¹²⁵]-Galanin binding activity. Radioligand was bound in buffer composed of 25 mM Hepes (pH 7.4), 2.5 mM MgCl₂, 100 mg/ml Baciacin and displaced in a dose dependent manner by unlabelled ligand. The assay was performed in triplicate for 3 hours at 4° C., and terminated by the addition of lysis buffer (48% urea, 2% NP-40 in 3M acetic acid). The concentration of radioligand in the assay corresponds to a final concentration of 20 pM.

Results and discussion—Binding of [¹²⁵I]-galanin to the galanin R1 receptor expressed in COS-7 cells is displaced by Cu(II) with an IC₅₀ of 28 ?M and a Hill coefficient of −4; whereas the 1,10-phenanthroline alone has no effect on [¹²⁵I]-galanin binding (FIG. I.2A). 1,10′-Phenanthroline binds Cu(II) with very high affinity, 7.6×10⁻¹⁰ M, and importantly, the Cu(II)-phenanthroline complex inhibits galanin binding with an affinity of 2 ?M i.e. 14-fold better than the free metal-ion. Mutational substitution identified residues Cys⁸⁸ (II:17) and Cys²⁹⁰ (VII:07) as being essential for the binding of the metal-ion complex (FIG. I.2B).

These experiments demonstrate, that a naturally occurring inter-helical metal-ion site in a 7TM receptor can be addressed by a metal-ion chelator complex with even higher affinity than by the free metal-ion.

### Example I.3

### Identification of Naturally Occurring Metal-ion Chelator Binding Site in the 12TM Dopamine Transporter

In the literature, a naturally occurring allosteric metal-ion binding site has been demonstrated in the dopamine transporter, a membrane protein having supposedly 12 transmembrane spanning segments, 12TM (Norregaard et al EGMO J. 17:4266-4273 (1998)). Here Zn(II) binds in a two-component fashion to a tridentate metal-ion site composed of residues His¹⁹³, His³⁷⁵, and Glu³⁹⁶ and thereby blocks dopamine transport. This effect of Zn(II) can be eliminated by mutational exchange of any of the three residues with a non-chelating residue.

Methods—The dopamine transporter cDNA was introduced into COS-7 by the standard calcium phosphate transfection method. Two days post-transfection the cells were assayed for [³H]-Dopamine uptake activity. The uptake assays was performed in 25 mM Hepes pH 7.4, 120 mM NaCl, 5 mM KCl, 1.2 mM CaCl₂, 1.2 mM MgSO₄, 1 mM ascorbic acid and 5 mM D-glucose and in the presence of various concentrations of unlabelled dopamine as indicated in the figures. The assay was performed in triplicate at 37° C. for 10 minutes, and terminated by washing with buffer twice and the addition of lysis buffer (48% urea, 2% NP-40 in 3M acetic acid).

Results and discussion—As shown in FIG. I.3A, 2,2′-bipyridine in complex with Zn(II) inhibits the transport of [³H]dopamine by the dopamine transporter, transiently expressed in COS-7 cells, in a two component fashion, i.e. with IC₅₀ values of 0.16 and 20 ?M, corresponding to a slightly higher potency than the free metal-ion, which similarly acts in a two component fashion, i.e. with IC₅₀ values of 2.2 and 338 ?M,. Importantly, the chelator bipyridine had no effect on the dopamine transport without being on complex with the metal-ion (FIG. I.3A). That the metal-ion chelator complex acts trough the same site as the free metal-ion was demonstrated by the mutational exchange of residue His¹⁹³ (FIG. I.3B). Dopamine transport could be inhibited also by a structurally distinct class of metal-ion chelators, exemplified by 2-pyridylamidoxime, O-acetyl (compound 210), which like 2,2′-bipyridine does not affect dopamine transport by itself, but blocks dopamine transport with a potency approx 10-fold higher than free Zn(II) and interestingly acts in a mono-component fashion (FIG. I.3C). This effect of the metal-ion chelator complex was eliminated by mutational substitution of His¹⁹³ known to be involved in metal-ion binding (FIG. I.3C). This substitution is known not to affect the transport of catecholamine Norregaard et al (1998) EMBO J. 17: 4266-4273) indicating that the effect of the metal-ion chelator complexes is mediated through the binding to a site (i.e. the endogenous metal-ion site), which is different from the catecholamine binding site. Thus, the metal-ion chelator complexes act as blockers of transport trough a novel allosteric molecular mechanism and could therefore serve as lead compounds in the development of a new type of transport blockers. It should be noted that the affinity of, for example 2-pyridylidoxinme, O-acetyl (compound 210) corresponds to even a very good lead compound found by simple screening.

The experiments presented in this section demonstrate that metal-ion chelator complexes of very different chemical structures can act as allosteric blockers of function—in these cases of either 7TM receptors or 12TM transporter proteins—through binding to naturally occurring metal-ion sites. Furthermore, it is shown that these compounds can bind with affinities similar to that of lead compounds found by conventional drug screening techniques. Thus, these metal-ion chelators can function as lead compounds in a chemical optimization process to obtain high affinity compounds acting as drug candidates.

II. Binding Of Metal-Ion Complexes In Engineered Metal-Ion Sites In Various Potential Drug Targets

Natural metal-ion sites are only found in a subset of potential drug targets. However, through mutagenesis it is possible to introduce metal-ion binding sites in proteins by introduction of metal-ion binding residues such as His, Cys, or Asp. The examples in the present section demonstrate how metal-ion complexes can bind to and affect the function of proteins after mutational engineering of metal-ion sites into the proteins.

### Example II.1

### Binding of Various Metal-ion Complexes to a Library of Inter-helical Metal-ion Sites Engineered into the NK1 Receptor

This example illustrates that different epitopes of a target protein—here a NK1 receptor—can be addressed by metal-ion chelator complexes, i.e. potential lead compounds for antagonists, after systematic mutational engineering of metal-ion sites into these different epitopes. Previously, a series of metal-ion sites have been built into the tachykinin NK1 receptor to probe helix-helix interactions, i.e. providing distance constraints in molecular models of the receptor (Elling et al. (1995) Nature 374: 74-77, Elling et al. (1996) EMBO J. 15: 6213-6219; Holst et al. (2000) Mol. Pharmacol. 58: 263-270). Here, such metal-ion sites are used as anchor points for potential lead compounds—i.e. metal-ion chelator complexes—for the development of receptor antagonists with different molecular mechanisms of actions.

Methods—The tachykinin NK1 receptor cDNA was expressed in COS-7 cells. Two days after transfection whole cells were assayed with respect to binding of radioactively labeled substance P ([¹²⁵I]-Bolton Hunter labeled Substance P), in displacement with substance P, ZnCl₂, CuCl₂ or various chelator complexes thereof present in a three fold molar ratio with respect to the metal-ion concentration. The zinc(cyclam) complex was prepared by co-incubation at 60° C. for one hour followed by overnight incubation at 37° C. The assay was typically performed in 12 or 24 well plates. On the day of assay, the cells were washed with binding buffer (50 mM Tris-HCl (pH 7.4), 150 mM NaCl, 5 mM nCl₂, 0.1% BSA, 0.1% and Bacitracin (100 mg/ml). Unlabelled competitor ligand and radioligand (20,000 cpm—approximately 20 pM) was added to the cells in binding buffer and incubation continued for 3 hours at 4° C. The assay was terminated by washing of the cells and lysis. The assay was performed in duplicate.

Results and discussion—Four different inter-helical metal-ion sites located between respectively TM-II and -III, TM-III and -V, TM-III and -VII, and TM-V and VI (FIG. II.1, Table in Panel A) were here probed with metal-ion chelator complexes in competition binding experiments against [¹²⁵I]-substance P in COS-7 cells transiently transfected with the NK1, receptor. An increase in affinity from approx. 10-fold to around 50-fold was observed in the metal-ion site engineered receptors as opposed to the wild-type NK1 receptor for free Zn(II) as well as for Zn(II) in complex with either 1,10-phenanthroline or in complex with 2,2′-bipyridine (FIG. II.1A). Thus, single to double digit ?M affinities were obtained for the metal-ion chelator complexes in these metal-ion site engineered receptors, corresponding to affinities observed for lead compounds in general found by conventional chemical screening. In the sites between TM-II and -III and between TM-III and -VII a similar increase in affinity was found for Cu(II) and Cu(II) in complex with the chelators as observed with the zinc-ions. However in the sites between TM-III and -V and between TM-V and -VI no increase or just a marginal increase in affinity was observed for copper and the copper-chelator complexes. Thus, different metal-ions can be exploited in different sites. In FIG. II.B is demonstrated that an inter-helical bis-His site, in this case constructed between TM-V and TM-VI, can also be addressed by a metal-ion chelator complex where the ion, in this case Zn(II), is bound in a circular chelator, here cyclam. Cyclam binds ZN(II) with a very high affinity, 3.2×10⁻¹⁶ M, which can be noted by the fact that the Zn(II)-cyclam complex has no effect on the wild-type NK1 receptor even at 10⁻³ M conc. i.e. an even smaller effect than the free metal-ion. Thus, the effect of the metal-ion chelator complex on the metal-ion site engineered receptor cannot be caused by the presence of free metal-ions.

The present example demonstrates that metal-ion chelator complexes can bind with suitable affinity, i.e. corresponding to ordinary lead compounds, in different parts of the main ligand-binding crevice of a 7TM receptor. This can be utilized, for example to target the lead compound and thereby subsequently the chemically optimized compound, i.e. the drug candidate, to bind and interact with different parts of the target molecule. In the present case, the metal-ion site between TM-II and -III can be used as anchor point for lead compounds addressing chemical interactions with wild-type residues located in the pocket between TM-I, -II, -III, and VII; whereas the metal-ion sites located between TM-III and -V and TM-V and -VI can be used as anchor points for chelating lead-compounds addressing residues in the pocket between TM-III, -IV, -V, -VI and -VII (see helical wheel diagram in FIG. II.1C). The metal-ion site located between TM-III and -VII may in principle be used to address either of these pockets. This approach can be used to deliberately direct the chemical optimization process, i.e. the molecular recognition towards specifically interesting parts of the target protein in order to obtain for example selectivity for a certain receptor subtype or a certain member of a family of related proteins. For example, families of monoamine and adenosine 7TM receptors are generally very highly—if not totally—conserved in the binding pocket for the natural ligand, i.e. the pocket between TM-III, -IV, -V, -VI, and -VII; however, they differ more in the pocket between TM-I, -II, -III, and VII. Conventional drug discover, methods are for various reasons highly biased towards the binding pocket for the natural ligand. The present approach allows for deliberate targeting of the lead compound and thereby also the final drug candidate for allosteric sites, i.e. pockets or epitopes distinct from the one used by the natural ligand.

### Example II.2

### Re-engineering of a Metal-ion Chelator Binding Site in the 12TM Dopamine Transporter

In example I.3, it was shown that the Zn(II)-bipyridine inhibited dopamine transport in a two-component fashion. This complicated type of interaction could hamper a subsequent further medicinal chemistry optimization of the chelator for high affinity interaction. In this example, the naturally occurring metal-ion site was re-engineered by elimination of one part of the metal-ion binding site and by introduction a new metal-ion binding residue.

Methods—as in example I.3.

Results and discussion—Re-engineering of the metal-ion site in the dopamine transporter was done by eliminating His¹⁹³, i.e. the residue found in the proposed extra-cellular loop 1, by substitution with a Lys residue and by introduction of an alternative metal-ion chelating His residue either in exchange for Glu³⁹⁶ located at the extra-cellular end of TM-8 or in exchange for Val³⁷⁷ located in TM-7. Both of these introduced His residues are located in a potentially favorable configuration for participating in metal-ion binding with His³⁷⁵ in TM-7, As shown in FIG. II.2, in both cases—[H193K;E396H] and [H193K;V377H]—more mono-component interaction curves were obtained for the metal-ion chelator complex in the re-engineered transporter mutants as compared to the wild-type transporter protein. This example demonstrates that a natural metal-ion site can successfully be re-engineered to create a less complex molecular or pharmacological phenotype. In a subsequent medicinal chemical optimization process such re-engineered metal-ion sites will be used in parallel with the natural site during the screening of chemical libraries.

In biological target molecules in general, more than one version of an engineered metal-ion site can in a similar fashion be used in parallel in the screening process in order to exploit the chemical libraries more efficiently. This approach enables each compound to contact, for example the same amino acid side chain located on an opposing transmembrane helix in more than one configuration.

The experiments presented in this section demonstrate that metal-ion chelator complexes can act as blockers of the function of biological target molecules—in these cases of either 7TM receptors or 12TM transporter proteins—through binding to metal-ion sites introduced by mutagenesis. Furthermore, these compounds can bind with similar affinity as lead compounds found by conventional drug screening techniques. Thus, these metal-ion chelators can function as lead compounds in a chemical optimization process to obtain high affinity compounds acting as drug candidates.

III. Increasing The Affinity/Potency Of The Metal-Ion Chelator Complexes Through Chemical Modifications Of The Chelator Molecule

In the present collection of examples, the mention chelators are considered as being bi-functional compounds, i.e., being composed of a metal-ion chelating moiety and a variable chemical moiety which interacts positively or negatively—depending on the chemical recognition—with spatially surrounding parts of the biological target molecule to which the chelator binds through either a natural or an engineered metal-ion site.

### Example III.1

### Structure-activity Relationship of Antagonist Metal-ion Complexes in the Galanin R1 and the Leukotriene LTB4 7TM Receptors

As discussed in examples I.1 and I.2, the human galanin receptor possesses a natural, antagonistic metal-ion site located between Cys⁸⁸ in TM-II and Cys²⁹⁰ in TM-VII, whereas the human leukotriene LTB4 receptor has a metal-ion site located between Cys⁹³ and Cys⁹⁷, both located in TM-III.

Methods—as in examples I.1 and I.2.

Results and discussion—A small library of commercially available 1,10-phenanthroline analogs in complex with Cu(II) were tested in competition for binding against [¹²⁵I]-galanin to the galanin R1 receptor expressed in COS-7 cells. This demonstrated, that many metal-ion chelator complexes bound with a similar affinity as the basic chelator compound, i.e. 1,10-phenanthroline, indicating that the modifications of the variable chemical moiety of the metal-ion chelator neither increased nor decreased the binding affinity (in FIG. III.1 is shown 5-phenyl-1,10-phenanthroline, compound 134, as an example). However, some chemical modifications clearly decrease the affinity of the metal-ion chelator complex, for example 2,9-dimethanol-1,10-phenanthroline (compound 133), or—importantly—some chemical modifications increase the binding affinity, for example 5-methyl-1,10-phenanthroline (compound 176) (FIG. III.1). In the LTB4 receptor similar results were obtained, however here different phenanthroline analogs yield different results. For example, the 5-phenyl substitution of phenanthroline (compound 134), which had no effect on the binding affinity in the galanin R1 receptor, entirely eliminated the binding of the metal-ion chelator complex in the LTB4 receptor (FIG. III.1).

### Example III.2

### Structure-activity Relationship of Antagonistic Metal-ion Complexes in the Metal-ion Site Engineered Tachykinin NK1 7TM receptor

The tachykinin NK1 receptor, which currently in the industry is a major putative target for the development of anxiolytic, antidepressive, as well as anti-emetic drugs, is here used as an example of a biological target molecule, in which an engineered metal-ion site can be used as an anchor point for the discovery and development of antagonistic drug candidates. As demonstrated in example II.1 a number of metal-ion sites could be built into the NK1 receptor and addressed by metal-ion chelator complexes competing for binding against radioactive substance P through interactions at different sites in the main ligand-binding pocket of the receptor, depending on the location of the metal-ion. Here, structure-activity relationships are demonstrated for a series of chelator analogs in two of these sites, i.e. the site between V:05 and VI:24 and the site between III:08 and VII:06.

Methods—as in example II.1.

Results and discussion—As observed in for example the galanin R1 receptor, many of the chemical variations of the variable part of the chelator were tolerated in the structure of the NK1 receptor when bound to the engineered metal-ion sites in complex with Zn(II). However, as demonstrated in FIG. III.2, clear differences were observed for some of the analogs in the two selected sites. Thus, 2,9-bis(trichloromethyl)-1,10-phenanthroline (compound 135) and 1,10-phenanthroline-5,6-dione (compound 175) bound 6- and 10-fold better than 1,10-phenanthroline in the [HisV:05,HisVI:24] site, but almost similar to 1,10′-phenanthroline in the [HisIII:08;CysVII:06] site—all in complex with Zn(II). In contrast the 5-phenyl-1,10-phenanthroline (compound 134) was 7-fold more potent in the [HisIII:08;CysVII:06] site than phenanthroline but only slightly more potent in the [HisV:05,HisVI:24] site—again all in complex with Zn(II). It should be noted here, that 5-phenyl-1,10-phenanthroline (compound 134) bound like 1,10′-phenanthroline in the galanin receptor, but was totally inactive in the leukotriene LTB4 receptor (see FIG. III.1.)

This example together with the previous example demonstrate, that relatively minor chemical modification of the variable, “non-metal binding” part of the chelator molecule can alter the recognition and antagonistic property of the metal-ion chelator complex both in biological target molecules having naturally occurring metal-ion sites as well as in molecules into which metal-ion sites have deliberately been engineered. Importantly, increases in affinities are observed demonstrating that the metal-ion chelators can be utilized as lead compounds in a drug discovery process towards high affinity compounds.

### Example III.3

### Structure-activity Relationship of Agonist Metal-ion Complexes in the Metal-ion Site Engineered Beta₂-adrenergic 7M Receptor

It is generally known in the field that while it is possible to find antagonistic lead compounds and optimize these for high affinity through medicinal chemistry efforts in many biological target molecules, it is generally much more difficult to find and develop agonist compounds, that is compounds, drug candidates, which activate the biological target molecule. The present example demonstrates how an engineered agonistic metal-ion site can be used as anchor-point for the development of agonists in a 7TM receptor.

Methods—Mutations were created in the beta2-AR cDNA by the PCR-directed overlap-extension method (Ho et al. (1989) Gene 77; 51-59). The beta2-AR cDNA was expressed by transient transfection into COS-7 cells. Two days after transfection the cells were assayed for intracellular levels of basal and ligand-induced cyclic AMP. The assay employed is essentially as described in Solomon et al (Anal. Biochem. (1974) 58: 541). Labelled adenine (2 ?Ci, [³H]adenine, Amersham TRK311) was added to cells seeded in 6-well culture dishes. The following day the cells were washed twice wit HBS buffer [25 mM Hepes, 0.75 mM NaH₂PO₄, 140 mM NaCl (pH 7.2)] and incubated in buffer supplemented with 1 mM 3-isobutyl-1-methylxanthine (Sigma I-5879). Agonists were added and the cells were incubated for 30 min at 37° C. The assay was terminated by placing the cells on ice and aspiration of the buffer followed by addition of ice-cold 5% trichloroacetic acid containing 0.1 mM unlabelled camp (Sigma A-9062) and ATP (Sigma A-9501). Cyclic AMP was then isolated by application of the supernatant to a 50W-X4 resin (BioRad) and subsequently an alumina resin (A-9003; Sigma) eluting the cyclic AMP with 0.1 M imidazole (Sigma I-0125). Determinations were done in duplicate.

Results and discussion—The inventors have previously demonstrated that Cys-substitution of Asn³¹² (AsnVII:06) in TMVII in the beta2-adrenergic receptor creates a bi-dentate metal-ion binding site with AspIII:08 at which metal-ion chelator complexes such as 1,10-phenanthroline and 2,2′-bipyridine in complex with either Zn(II) or Cu(II) can bind and act as agonists for the receptor (Elling et al. PNAS 1999, 15: 6213-6219). As shown in FIG. III.3B an extended version of this site including also a substituted residue, Phe²⁸⁹ (PheVI:16) located in the important TM-VI, metal-ion chelator complexes, in this case Cu(II)-1,10-phenanthroline and Cu(II)-bipyridine display higher agonistic efficacy than in the TM-III to TM-VII site. The free metal-ion or the chelator by itself has no stimulatory effect in the metal-ion-site engineered receptor (FIG. III.3B). That the agonistic effect of the metal-ion chelator complex is not caused by some kind of covalent modification of the receptor—for example oxidation—is shown in FIG. III.3, where a simple washing experiment demonstrates how the stimulatory effect quickly disappears, when the metal-ion chelator is removed, while the stimulation continues if the metal-ion chelator complex is re-added. When a library of bipyridine analogs were tested for agonistic activity in this site, many were found not to be active (data not shown), while some were shown to be as potent as bipyridine itself (FIG. III.3C). Importantly, a compounds such as 2,2′-di(benzimidazol-2-yl)-quinoline), (compound 85) was found to stimulate signal transduction as determined in cAMP accumulation in the metal-ion site engineered receptor with an x-fold improved potency, i.e. EC₅₀=470 nM.

This example demonstrates, that the variable, non-metal-ion binding part of the chelators can be modified to create nanomolar affinity agonists in metal-ion site engineered biological target molecules, Such a compound could serve as an intermediate “chemical stepping-stone” in the process of developing high affinity agonists for the metal-ion site engineered receptor. And, similarly agonistic metal-ion sites can be engineered into other 7TM receptors and other biological target molecules in general to serve as anchor points for the initial identification as well as the initial optimization process for agonist leads for such target molecules.

### Example III.4

### Structure-activity Relationship of Antagonistic Metal-ion Complexes in a Soluble Proteins the Enzyme FVIIa

The previously presented examples have all represented membrane proteins which obviously constitute a very large group of biological target molecules for medical drugs. In the present example, Factor VIIa, i.e. the active form of the FVII protease involved in the coagulation cascade is used to demonstrate that metal-ion chelator complexes can modulate the function of a soluble protein, in this case an enzyme which is known to possess an appropriate, allosteric metal-ion site (Dennis et al. Nature (2000) 404: 465-470).

Method—The amidolytic activity of Factor VIIa (FVIIa) was measured by the incubation of 2.5 μl FVIIa (100 nM final concentration, obtained from American Diagnostica), 2.5 μl ligand and 4 μl substrate (10 mM, S2288 obtained from Chromogenix) in 42.5 μl buffer (50 mM Hepes pH 7.4, 1 mM CaCl₂, 100 mM NaCl, 0,02% Tween 20). The assay was performed in 96-well plates (Costar). Incubation was performed at room temperature for five hours with absorbance read every 10 minutes.

Results and discussion—As shown in FIG. III.4A, 2,2′-bipyridine without metal-ions has no effect on the activity of FVIIa; however in complex with Zn(II), 2,2′-bipyridine inhibits the enzymatic activity with a 100 ?M affinity. Many bipyridine analogs act with a similar potency as the basic chelator, however for example Zn(II)-4,4′-di-terbutyl-2,2′-dipyridyl (compound 180) inhibits FVIIa enzyme activity with an 8.5-fold increased potency as compared to Zn(II)-bipyridine (FIG. III.4.A). In contrast Zn(II)-4,4′-di-terbutyl-2,2′-dipyridyl (compound 180) inhibits LTB4 binding to the LTB4 receptor with a potency which is 10-fold lower than Zn(II)-bipyridine alone (FIG. III.4B). As shown in FIG. III.4C, 1,10-phenanthroline had no effect on FVIIa activity by itself however in complex with Zn(II) 1,10-phenanthroline inhibits the enzyme activity with a potency of 110 ?M. As with 2,2′-bipyridine, many phenanikoline analogs act with a potency similar to or lower than 1,10′-phenanthroline itself (data not shown); however, for example 2,9-bis(trichloromethyl)-1,10-phenanthroline in complex with Zn(II) inhibits FVIIa activity with increased potency as compared to Zn(II)-1,10′-phenanthroline (FIG. III.4C).

Most enzyme inhibitors act by binding at—or near by—the active site of the target molecule. However, as recently demonstrated for FVIIa, very efficient inhibition can be obtained also by binding instead at exosites or allosteric sites located far away from the active site in the biological target molecule (Dennis et al. Nature (2000) 404: 465470). The method described here can be utilized to specifically target the lead compound and thereby the final drug candidate to act at allosteric sites in the target molecule, as the binding site is determined by the site at which the anchoring metal-ion site is engineered. Inhibition of enzymes and proteins in general at allosteric sites is particularly interesting since the active site often is relatively similar in enzymes belonging to a particular protein family, for example kinases or phosphatases, which means that it can be difficult to obtain selectivity of drugs acting at the active site. This is not the case with drugs acting at allosteric sites.

### Example III.5

### Structure-based Optimization of Metal-ion Chelators for Secondary Interactions in the CXCR4 Receptor and Other Biological Target Molecules

The previous examples in this session have demonstrated, that it is possible to obtain both decreased, but importantly, also increased affinity by modifying the variable, non-metal binding part of metal-ion chelators, which in various biological target molecules bind to either natural or engineered metal-ion sites. These examples were gathered mainly from screenings of commercially available, small libraries of chelator analogs. In the present example it is described how the process of increasing the affinity or potency of the metal-ion chelator can be performed in a deliberate structure based fashion in this case through the establishment of a charge-charge interaction. The metal-ion-mediated binding of the metal-ion chelator is here considered as being the “primary interaction point” or the anchor point, while the subsequent establishment of other chemical interactions is considered to be “secondary interaction points”.

Methods—The cDNA coding for, for example the CXCR4 chemokine receptor can be expressed in COS-7 cells as described for other 7TM and 12TM proteins previously. Metal-ion sites may be engineered through PCCR-directed mutagenesis and the functional activity of the receptor be tested for instance by (established) binding experiments employing the radiolabelled ligand, [¹²⁵I]-SDF1α.

Results and discussion—The inventors have demonstrated that Asp¹⁷¹ (AspIV:20) located at the extracellular end of TM-IV on the face pointing inwards, towards the main ligand binding crevice of the CXCR4 receptor is exposed and can be used as attachment site for the positively charged cyclam ring of non-peptide bicyclam antagonists for this receptor. Metal-ion binding sites will be introduced in the CXCR4 receptor in the spatial vicinity of AspIV:20 by introduction of a His residue at position V:01 which will form a bis-His metal-ion binding site with the naturally occurring HisIII:05 in the CXCR4 receptor—as previously demonstrated in the NK1 receptor (Elling et al. EMBO J. (1996) 15: 6213-6219). Similarly an intra-helical bis-His site will be introduced between residues V:01 and V:05 through introduction of two His residues at these positions and between III:05 and IV:24 through His substitution at position IV:24. Thus three metal-ion sites will be constructed all within few Å's of AspIV:20 (see helical wheel diagram in FIG. III.5). A small library of 1,10-phenanthroline analogs will be obtained or synthesized in which amino-methyl, amino-ethyl, amino-propyl, and aminobutyl will be placed in either the 2, 3, 4, or 5 positions and a similar small library where the sane substituents will be placed in either the 3, 4, or 5 position of bipyridine will similarly be constructed. In a typical experiment, these libraries of amino-substituted chelators will be tested in complex with either Zn(II) or Cu(II) in the metal-ion-site engineered CXCR4 receptors, and the compounds ability to inhibit the binding of ¹²⁵I-SDF1? or the binding of [¹²⁵I]-12G5 monoclonal antibody or the ability of the compounds to inhibit the signal transduction mechanism induced by SDF-1a will be tested as performed for metal-ion chelators in the previous examples described above. Due to the spatial proximity as well as the relative conformational flexibility of the system, several of these compounds will in several of the sites have the opportunity of forming a salt-bridge between the amino function of the amino-substituted metal-ion chelator and the carboxylic acid function of Asp¹⁷¹ (AspIV:20). This formation of a secondary interaction will be quantified as an increased affinity or an increased potency of the metal-ion complex of the amino-substituted chelator in comparison to the corresponding metal-ion complex of the non-substituted phenanthroline or dipyridine. Due to the relatively high energy in the charge-charge-interaction a considerable increase in affinity or potency will be observed. The molecular interaction mode of the amino-substituted chelator(s) will be confirmed through mutational substitutions of Asp¹⁷¹ with Asn, Ala and other residues. Depending on the structure of the most optimal amino-substituted analog(s) a second and third round of analogs will be synthesized which conceiveably will present an appropriate basic moiety in a more conformationally constrained fashion.

These mini-libraries of amino-substituted metal-ion chelators can be utilized in several biological target molecules, which present Asp or Glu residues in an appropriate fashion. For example, in the CXCR4 receptor Asp²⁶² (AspVI:23) is equally available as Asp¹⁷¹ for interaction as previously described (Gerlach et al.). Similarly AspIII:08 is conserved among monoamine receptors and, for example opioid and somatostatin receptors and this residue is a known interaction point for amine functions (Strader et al (1991) 266: 5-8). These and other acidic, potential secondary interaction points for amino-substituted metal-ion chelators can be addressed through construction of a small number of metal-ion sites placed in their spatial vicinity—as described above for Asp¹⁷¹ (AspIV:20). Similarly amino-functions in a biological target molecule—for example, epsilon amino groups of Lys residues—can be addressed by, for example mini-libraries of tetrazol substituted metal-ion chelators. As described, charge-charge interactions will initially be pursued for establishing secondary interactions for the metal-ion chelator lead compounds. However, other types of weaker interactions such as hydrogen-bonds, amino-aromatic interactions, aromatic-aromatic interactions, aliphatic hydrophobic interactions, van der Walls interactions etc. will also be exploited in a similar, systematic fashion as described above for the charge-charge interactions.

In the present section, a 7TM receptor is for convenience used as an example of a biological target molecule. In this system, very useful molecular models are available, which have been refined and have allowed for, for example the construction of intra- and especially inter-helical metal-ion sites. However, due to lack of, for example an array of suitable X-ray structures of this or similar targets in complex with agonists and antagonists it is not possible to apply classical structure-based drug design methodology in full. Nevertheless, for example in these membrane proteins the present method does to a certain degree compensate for the lack of knowledge of the detailed 3D stucture of the target molecule by anchoring the lead compound and thereby creating a fix-point for the subsequent medicinal chemical optimization point guided by the molecular models.

The approach described above could be further helped and guided by detailed knowledge of the 3D structure(s) of the biological target molecule, preferentially determined in complex initially with the un-substituted metal-ion chelator and subsequently in complex with the chemically modified metal-ion chelator in which attempts have been made to establish first one secondary interaction and subsequently further secondary or tertiary interactions. For some biological target molecules such as soluble proteins this can be achieved through for example crystallization and standard X-ray analysis procedures or through, for example NMR analysis of the complex in solution again using standard procedures. Here, the method can take advantage of methods developed for structure-based drug discovery in general. This would make it possible to apply classical structure-based approaches such as structure-based library design for the establishment of secondary and tertiary interaction sites for the lead compound in the target molecule. However, it should be noted, that a major advantage and difference of the present method is, that the lead compound is anchored to a particular site and thereby to a certain degree in a particular conformation in the biological target molecule through binding to the bridging metal-ion site while the compound is being optimized for chemical recognition with the target molecule.

Also it should be noted that through the application of a more-or-less flexible spacer in between the metal-ion chelating moiety and the so-called variable chemical moiety of the test compound it becomes possible to probe for interaction or binding to structurally and functionally interesting epitopes of the biological target molecule with variable chemical moieties, which due to their intrinsic low affinity would not be detectable in the analytical systems on their own; but, which—due to the local high concentration of these created by the binding of the tethering metal-ion chelating moiety to the metal-ion site—now are detected.

### Example III.6

### Structure-based Optimization of Metal-ion Chelators to Use as Antagonists in “Pharmacological knock-out” Experiments

The approach described in the previous examples will be used as (a) step(s) in the drug development process in general to increase the affinity of lead compounds for the biological target molecule through establishment of chemical recognition between the ligand and structural elements found in the wild-type target molecule, i.e. in the unmodified vicinity of the engineered metal-ion site. However, the method will also be used for example to increase the affinity and specificity of metal-ion chelator compounds to be used in pharmacological knock-out applications. This procedure has in principle been described previously (Elling et al. (1999) Proc. Natl. Acad. Sci. USA 96:12322-12327); however only for basic metal-ion chelating agents. Briefly, the method is based on the introduction of a silent metal-ion site in a potential drug target, i.e. creation of a metal-ion site in which the mutations do not affect the binding and action of the endogenous ligand for the receptor. When such a metal-ion site engineered receptor is introduced into an animal by classical gene-replacement technology, i.e. exchange of the endogenous receptor with the metal-ion site engineered receptor, then the animals will develop normally without any development of compensatory mechanisms, which otherwise frequently impair the interpretations of the phenotypes in classical gene knock-out technology. In the adult animals or whenever it is found appropriate the animals are then treated with an appropriate metal-ion-chelating agent which then will act as an antagonist and turn off the function of the metal-ion site engineered receptor. Currently, this approach is impaired by the fact, tat the generally available metal-ion chelating agents only will bind with at best ?M affinity to the metal-ion site engineered biological target molecule, which will give similar ?M or lower antagonistic potencies. These relatively low potencies and the relative low specificity of the basic test compounds impairs the general applicability of the technology due to simple pharmacokinetic and toxicology problems.

By applying the technology described in the previous example and in the previous examples in general, it will be possible to increase the affinity of metal-ion chelators significantly, which will make it considerably more easy to reach therapeutic, efficient antagonistic concentrations of the metal-ion chelator in the animals and also to increase the “therapeutic window” due to the higher degree of selectivity of the compounds caused by the establishment of more than one molecular interaction point. Establishment of just a single suitable charge-charge interaction will increase the affinity of the metal-ion chelator by 10 to 100-fold or more. This will be performed as an example in the so-called RASSL a modified kappa-opioid receptor, which previously has been used in gene-knock out experiments (Redfern et al. Nat. Biotechnol. (1999) 17:165-169). By introduction of metal-ion sites, for example between TM-V and TM-VI or between TM-VI and TM-VII or between TM-II and TM-III or between TM-III and TM-VII in a kappa-opiod RASSL molecule and through screening of, for example the mini-library of amino-substituted metal-ion chelators it will be possible to select a nano-molar affinity antagonist because of the formation of a secondary charge-charge interaction with AspIII:08, i.e. the Asp in TM-III corresponding to the amine-binding Asp in monoamine receptors.

IV. Optimization Of Compounds On The Wild-Type Biological Target Molecule

In the case, where the initial binding of the metal-ion chelator was obtained through mutational introduction of an anchoring metal-ion site in the biological target molecule, a final step of optimization will have to be performed to obtain high affinity binding or potency on the wild-type target molecule without the metal-ion bridge. Through the methods described in the previous experiments, the metal-ion chelator lead compound will gradually be optimized for interactions with chemical groups in the biological target molecule spatially surrounding the metal-ion site—i.e. interactions with chemical groups found also in the wild-type target molecule. Thus, the test compound will gradually increase its affinity not only for the metal-ion site engineered molecule but also for the wild-type biological target molecule. When two to three secondary interaction points have been established, the affinity of the test compound for the wild-type target molecule, which is being tested in parallel with the metal-ion site engineered molecule, will have reached micro-molar affinities, i.e. a lead compound on the wild-type target molecule has been created. At this point one or more of the following three approaches will be followed: 1) structure-based further chemical optimization of the compound in general aiming at improving recognition at various known chemical moieties of the target molecule; 2) structure-based further chemical optimization of the compound at which the “metal-ion site bridge” is exchanged by a more classical type of chemical interaction with the residue(s) which had been modified to create the metal-ion site in the biological target molecule. Here advantage can be taken of the fact that the geometry of the metal-ion site anchor is well known in general and, that relatively limited structure-based libraries can be established to create a new type of interaction; 3) further chemical optimization of the compound through more-or-less random generation of chemical diversity in general in the compound.

The above-given examples describe specific methods that can be employed to practice the present invention. Based on the details given a person skilled in the art will be able to devise alternative methods at arriving in the same information using the concept of the invention. However, the examples are not to be construed to limit the invention in any way.

### APPENDIX I

### Patent EXAMPLES—FIGURES

I. BINDING OF METAL-IONS AND METAL-ION COMPLEXES TO VARIOUS DRUG TARGETS WITH NATURAL METAL-ION SITES

L.1 Identification of naturally occurring metal-ion chelator binding site in the 7TM leukotriene LTB4 receptor

L.2 Identification of naturally occurring metal-ion chelator binding site in the 7TM galanin receptor

L.3 Identification of naturally occurring metal-ion chelator binding site in a 12M protein, the dopamine transporter.

II. BINDING OF METAL-ION COMPLEXES IN ENGINEERED METAL-ION SITES IN VARIOUS POTENTIAL DRUG TARGETS.

II.1 Binding of various metal-ion complexes to a library of inter-helical metal-ion sites engineered into the tachykinin NK1 receptor

II.2 Re-engineering of a metal-ion chelator binding site in the 12TM dopamine transporter.

III. INCREASING THE AFFINITY/POTENCY OF THE METAL-ION CHELATOR COMPLEXES THROUGH CHEMICAL MODIFICATIONS OF THE CHELATOR MOLECULE.

III.1 Structure-activity relationship of antagonist metal-ion complexes in the galanin and the leukotrien LTB4 receptors.

III.2 Structure-activity relationship of antagonistic metal-ion complexes in the metal-ion site engineered tachykinin NK1 receptor.

III.3 Structure-activity relationship of agonist metal-ion complexes in the metal-ion site engineered beta2-adrenergic 7TM receptor.

III.4 Structure-activity relationship of antagonistic metal-ion complexes in a soluble protein, the enzyme FVIIa.

III.5 Structure-based optimisation of metal-ion chelators for secondary interactions in the CXCR4 receptor and other biological target molecules.

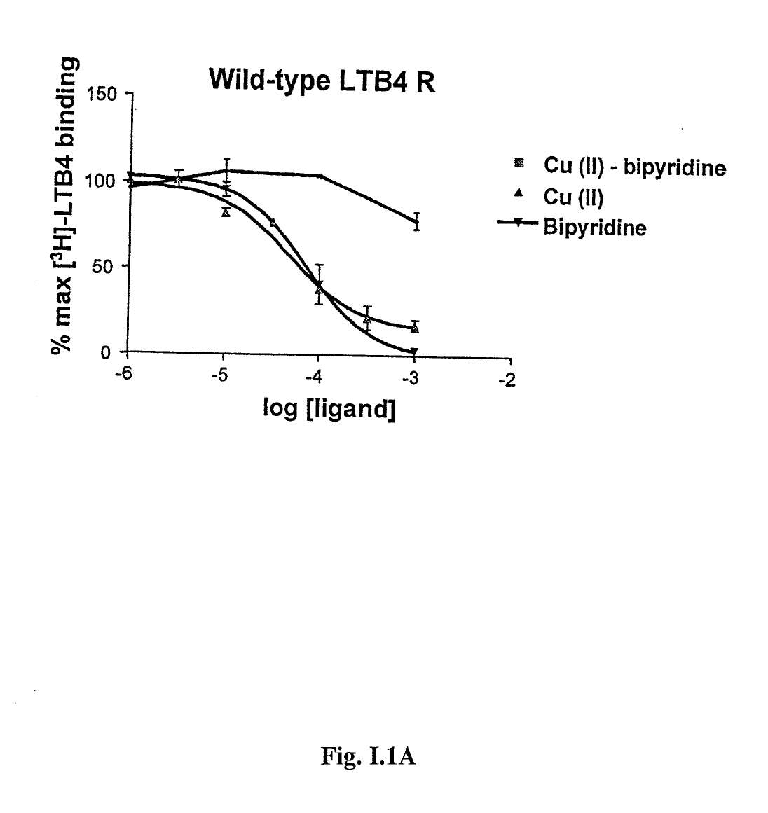

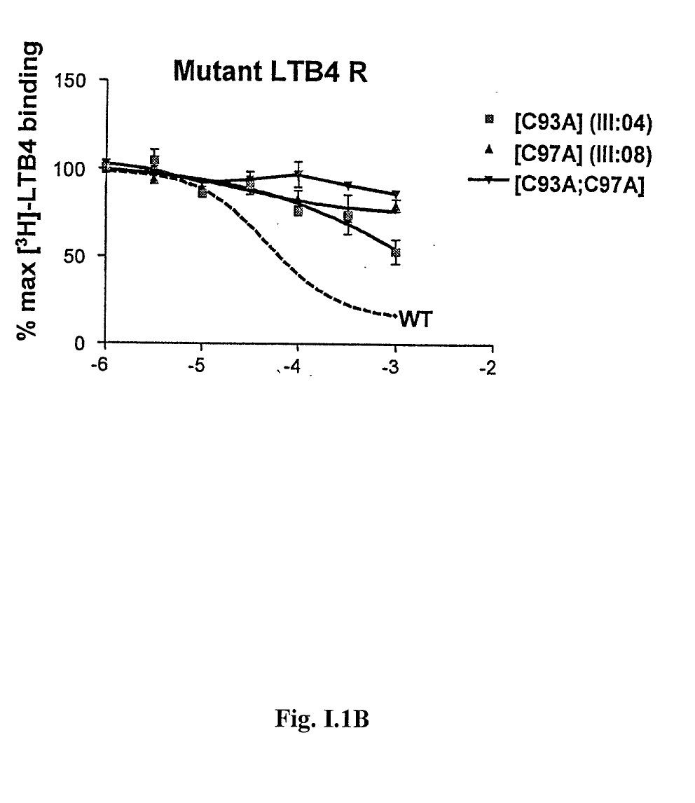

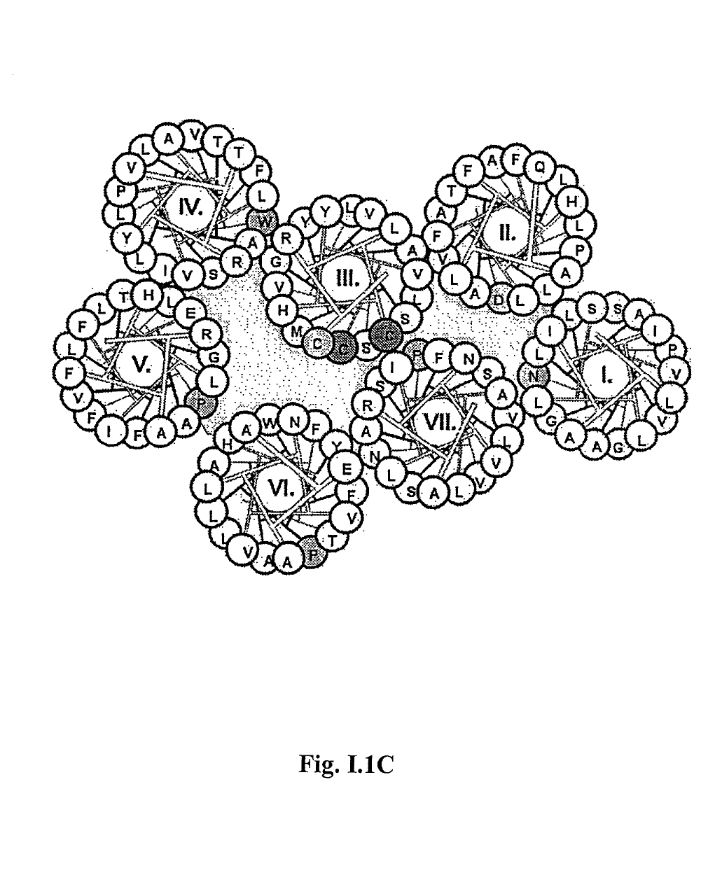

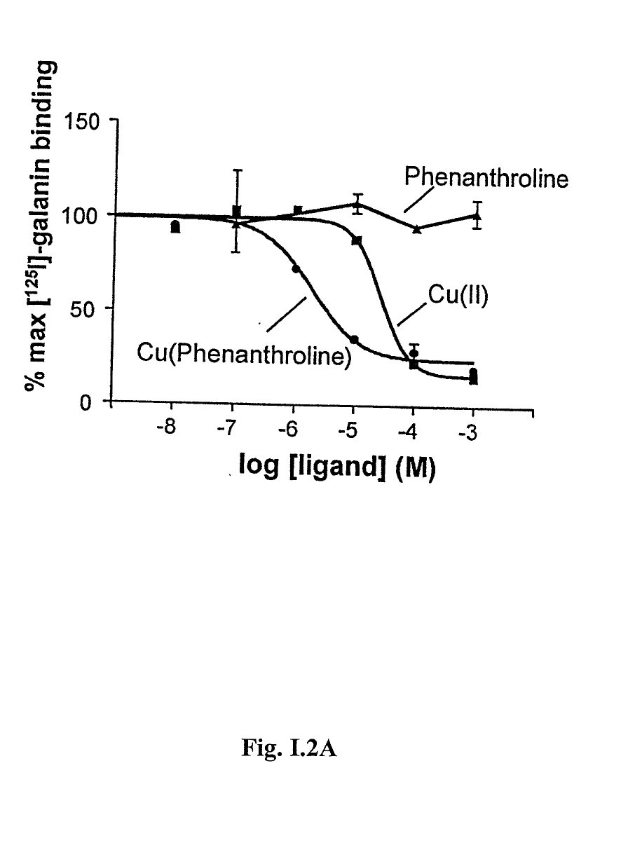

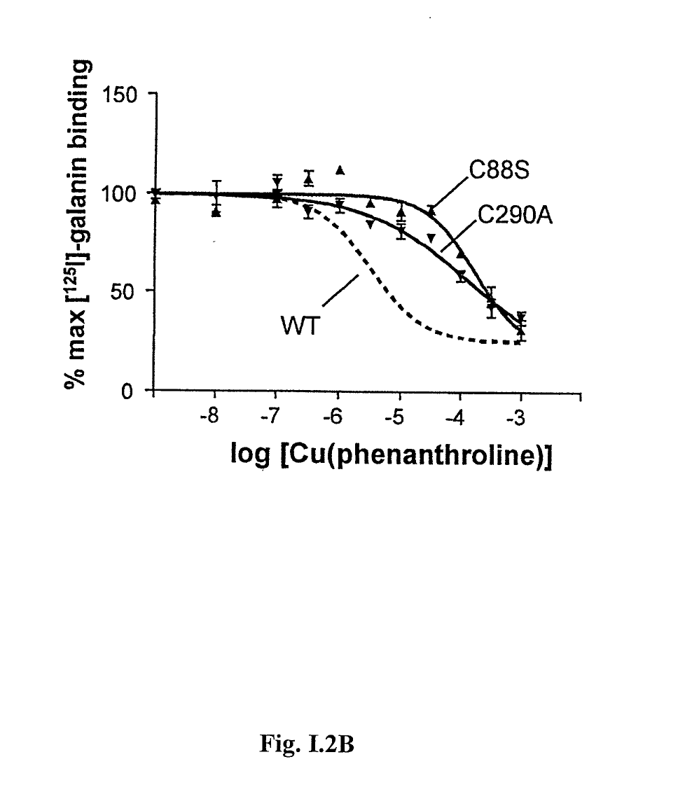

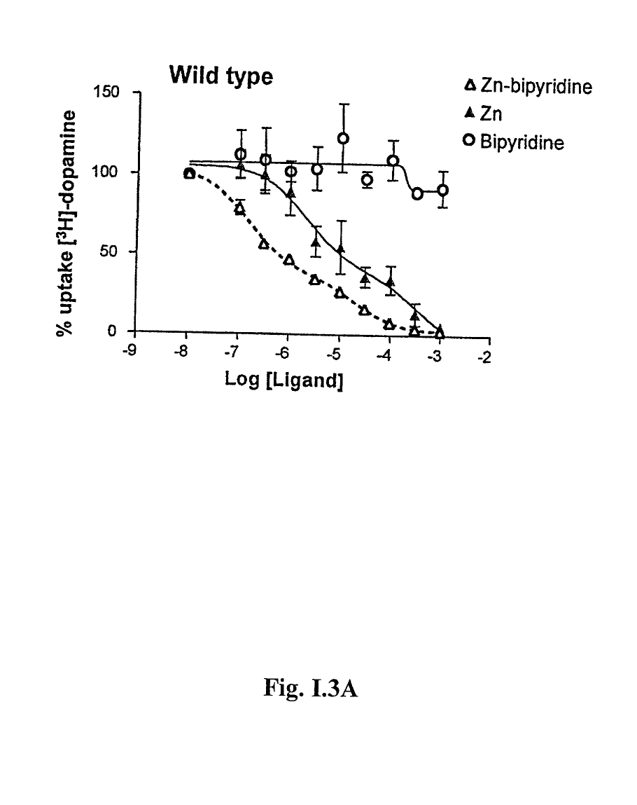

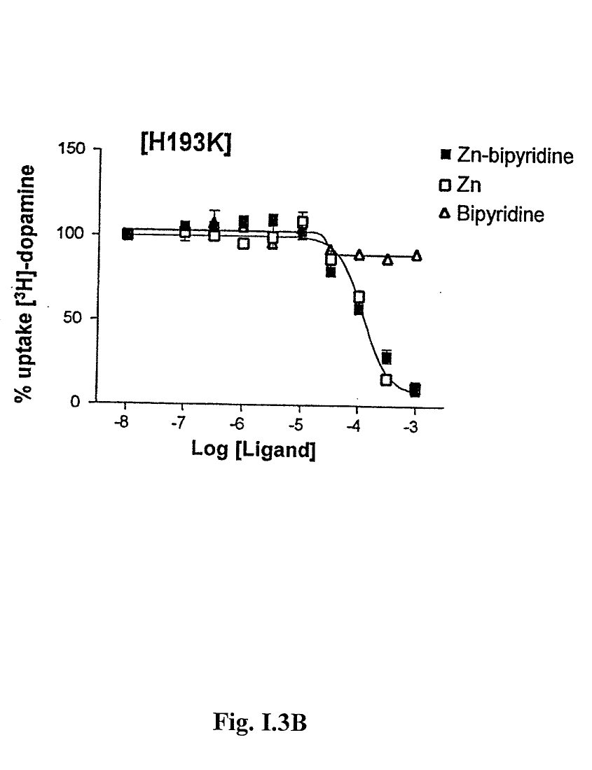

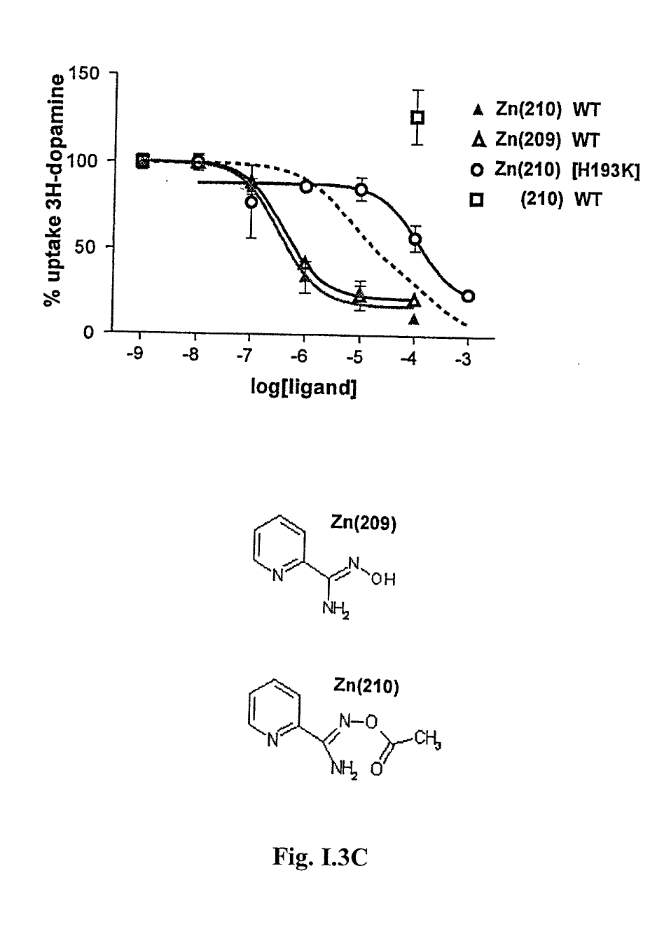

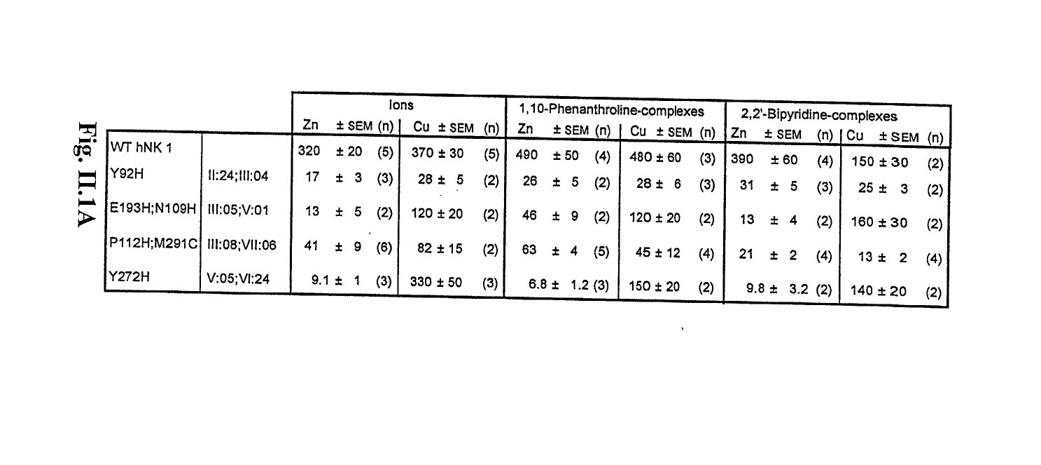

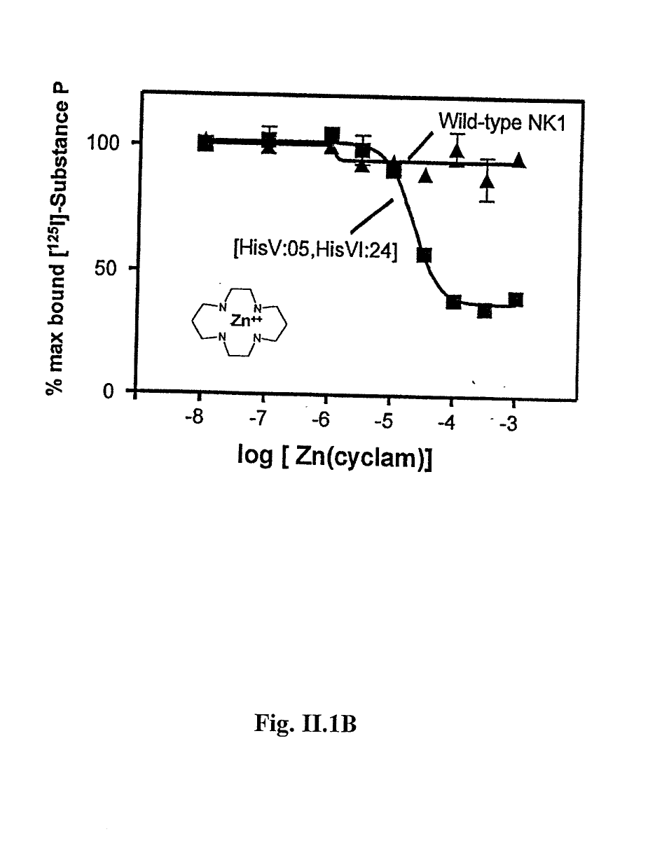

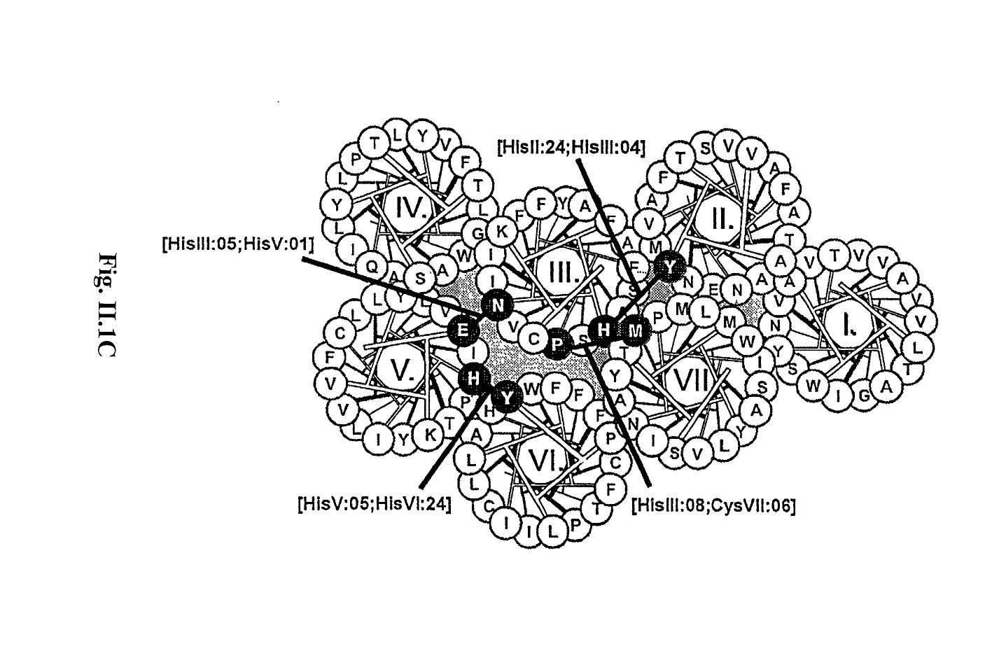

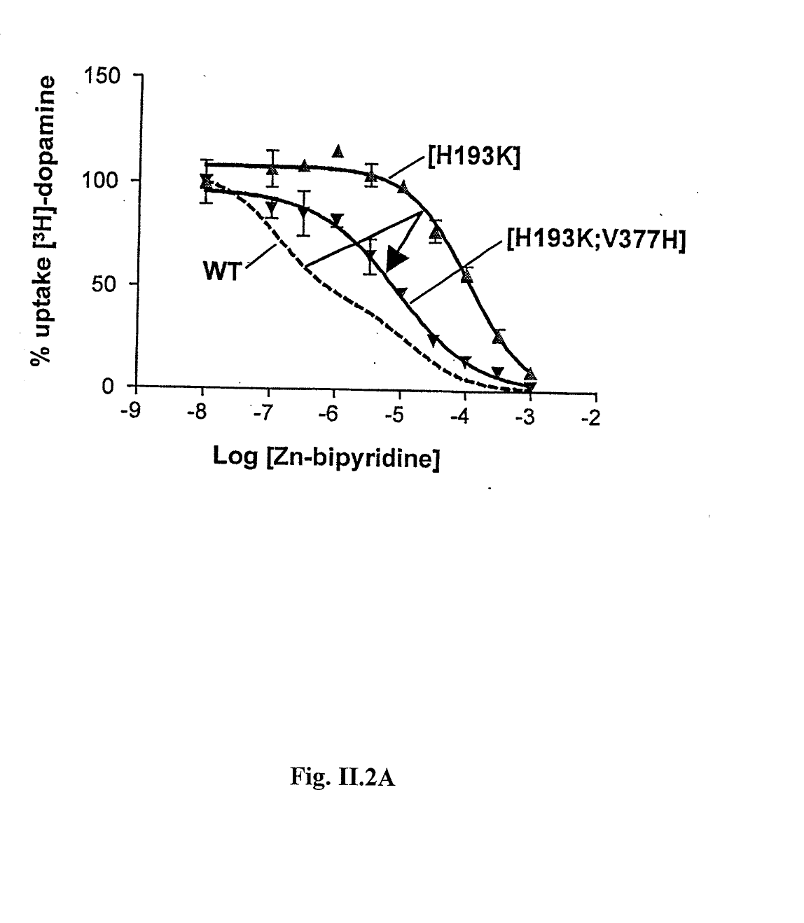

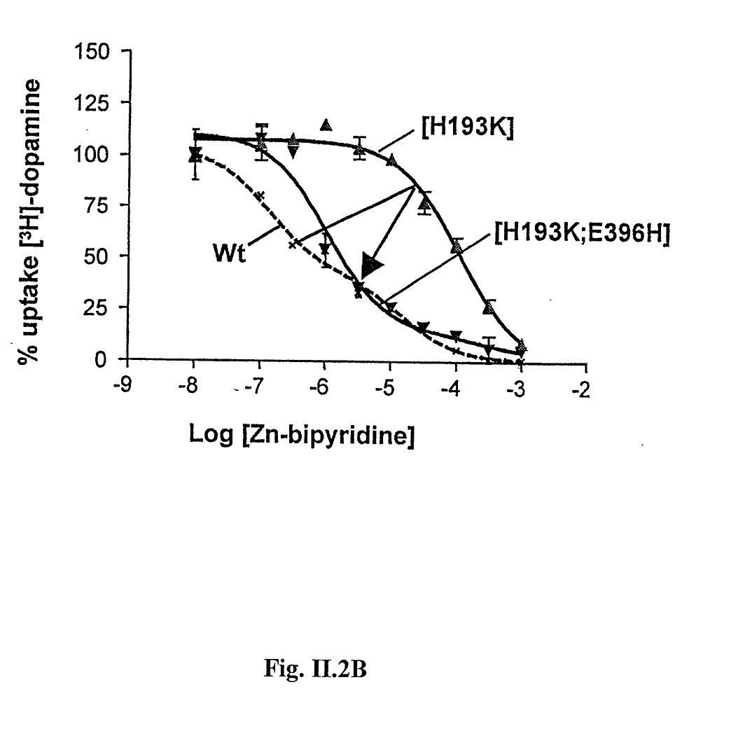

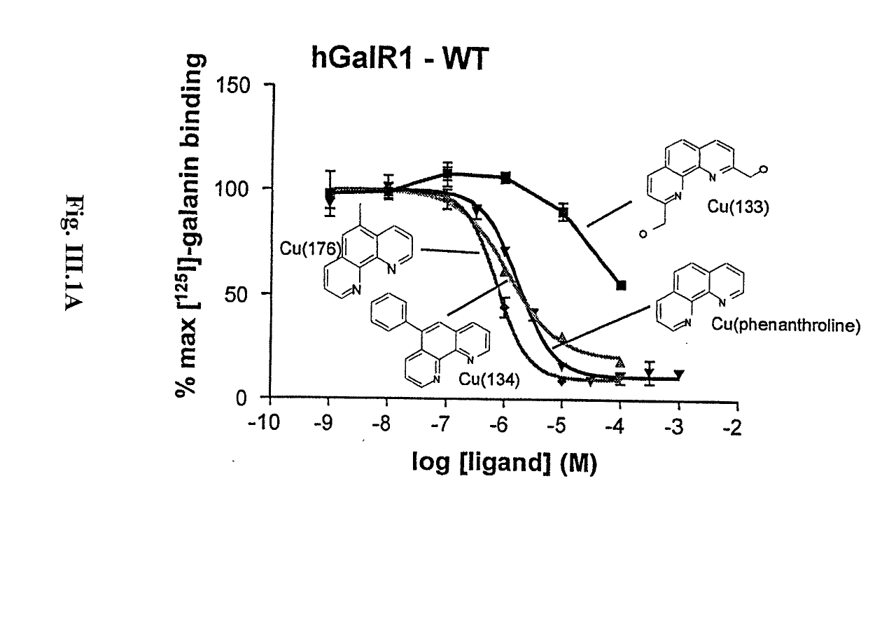

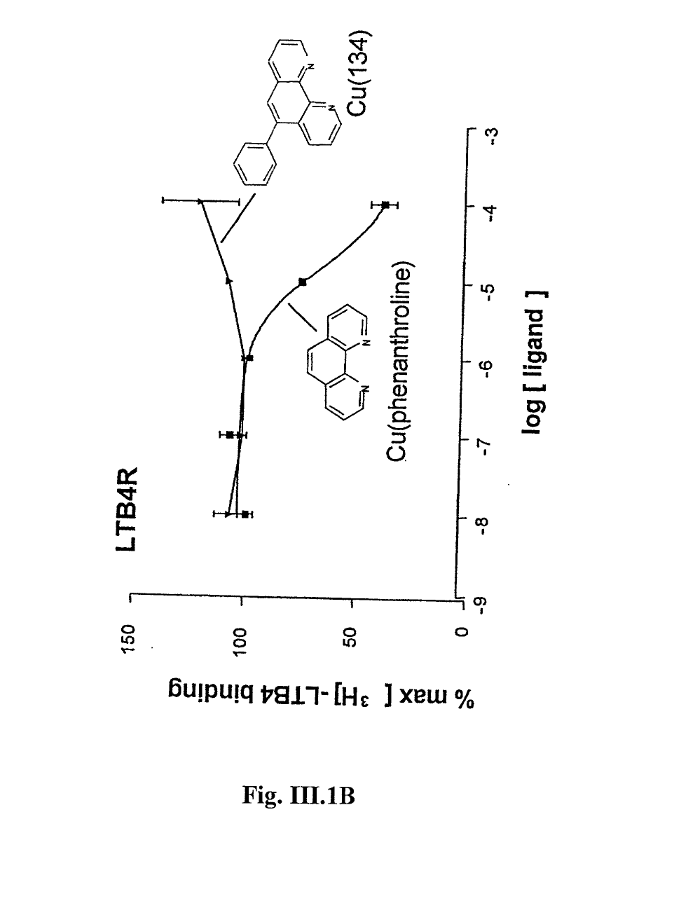

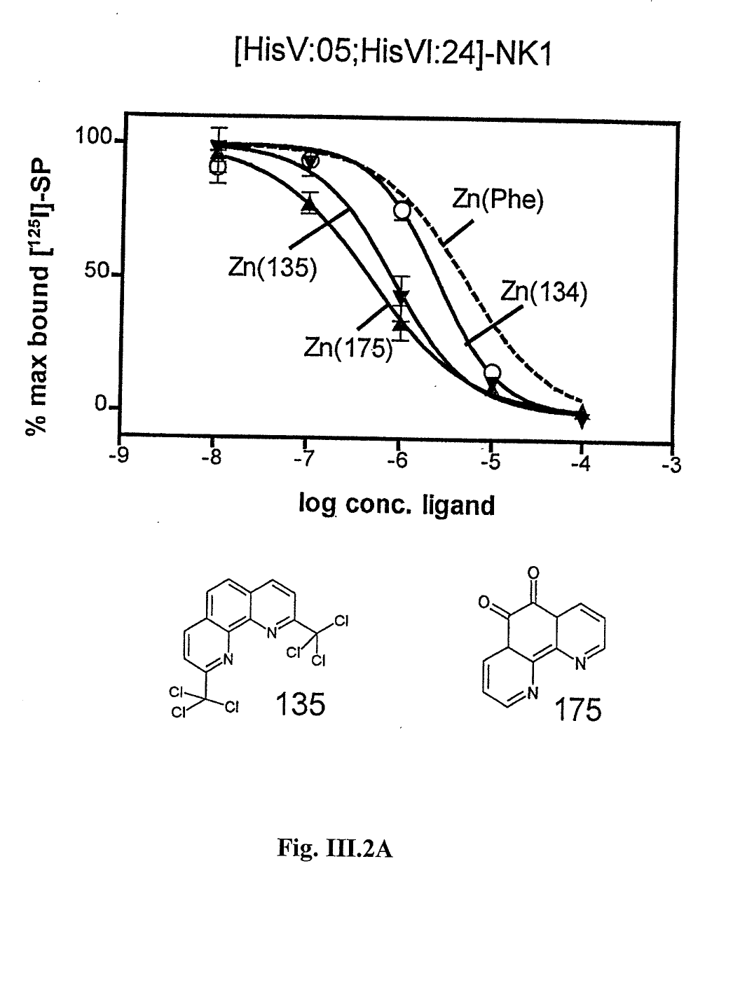

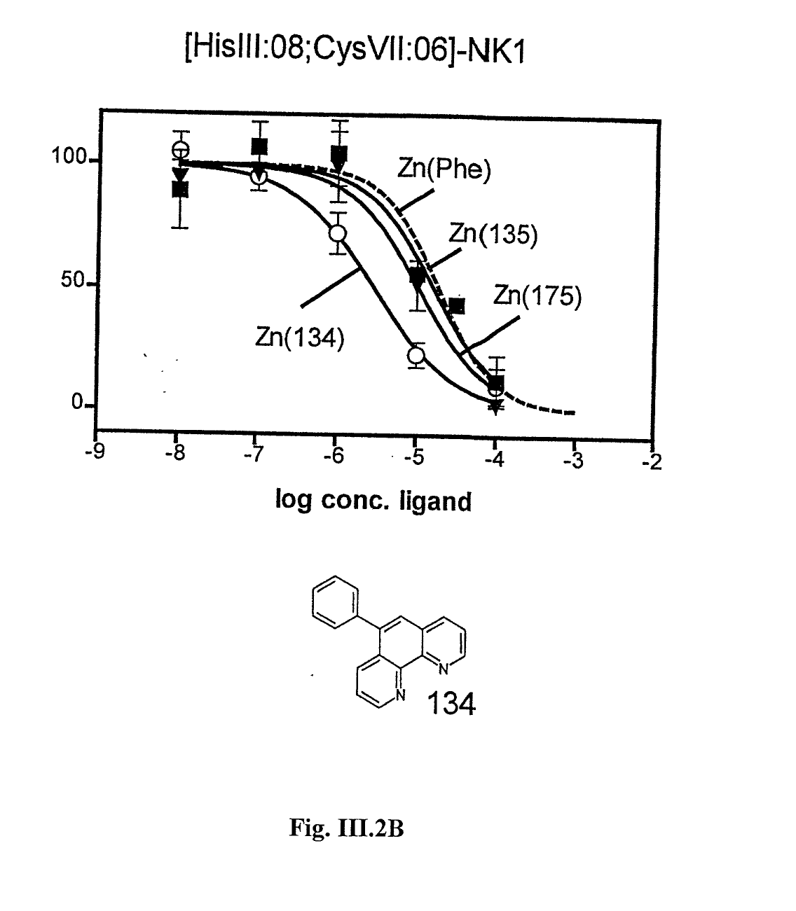

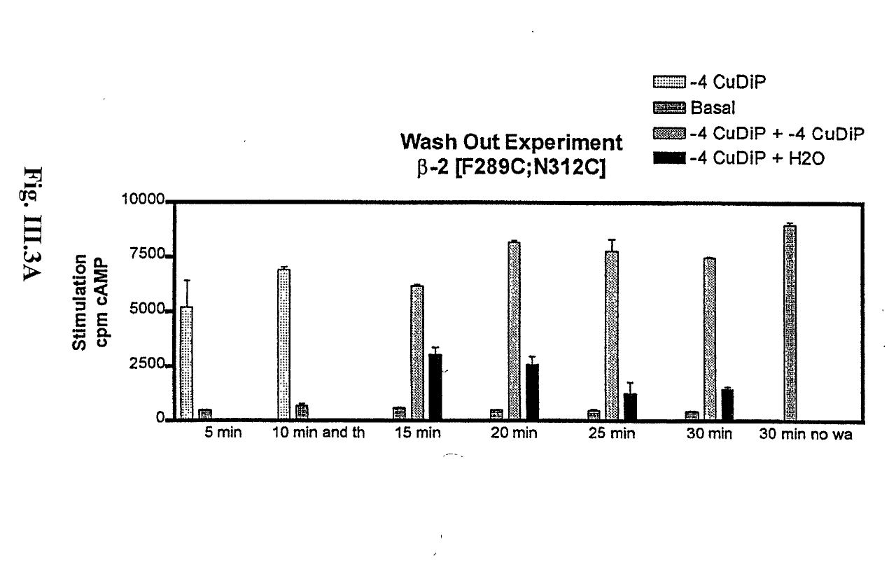

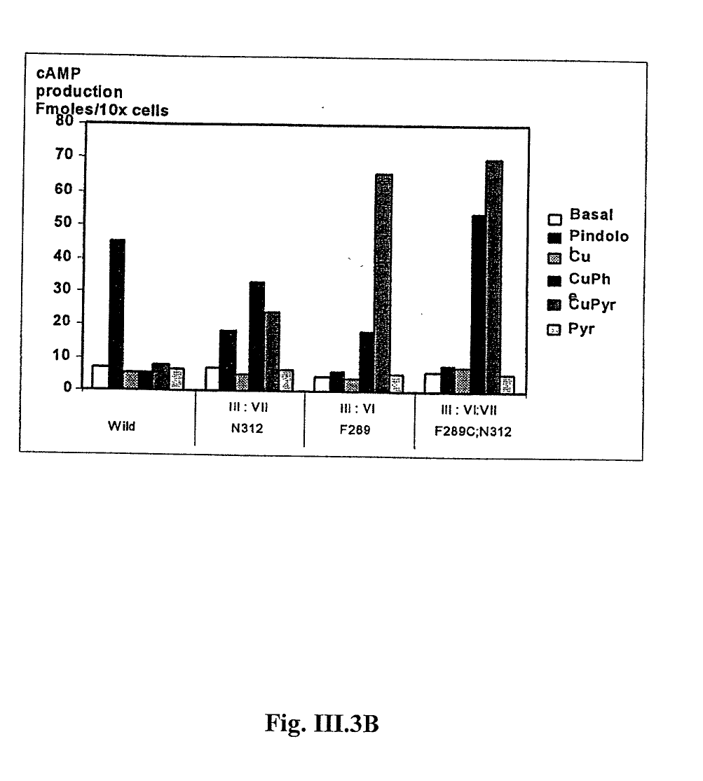

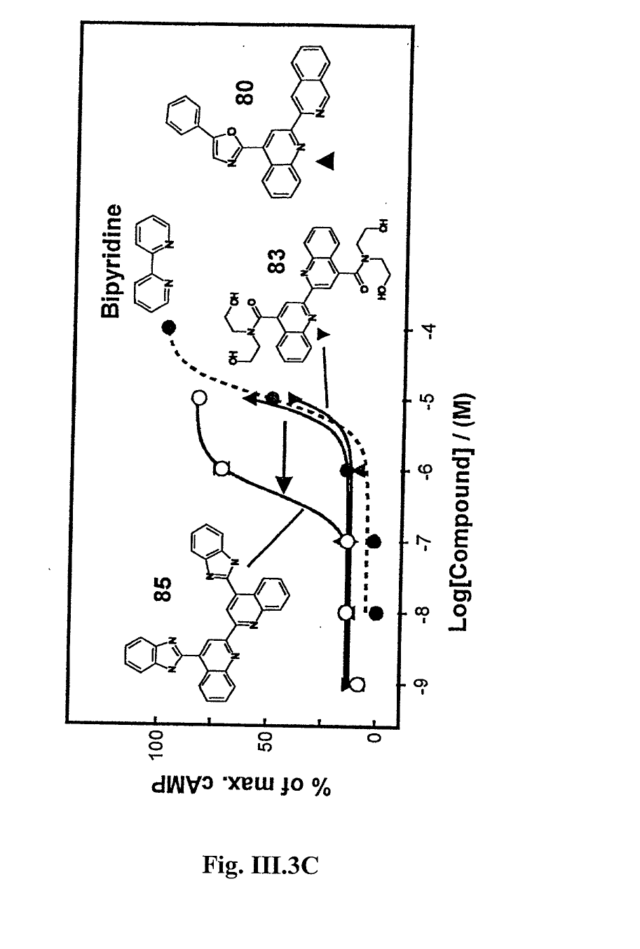

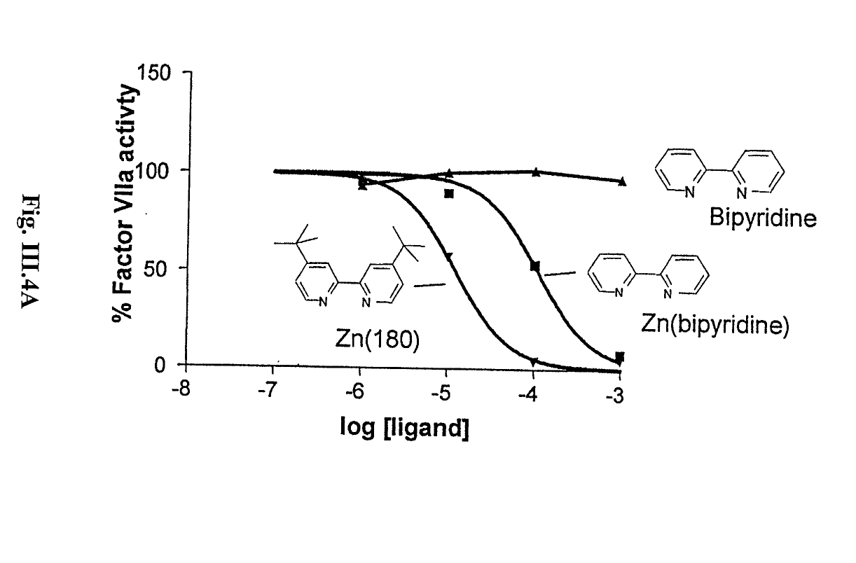

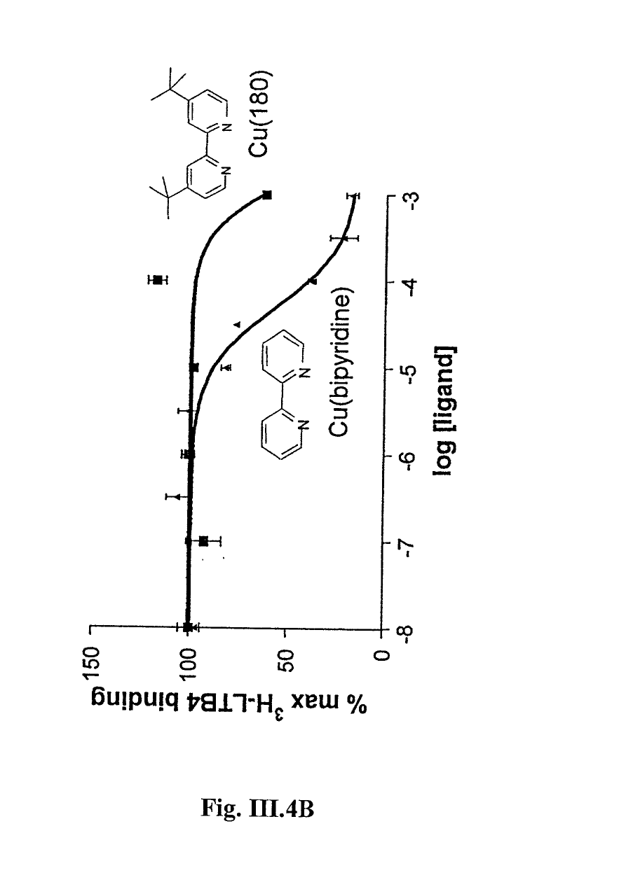

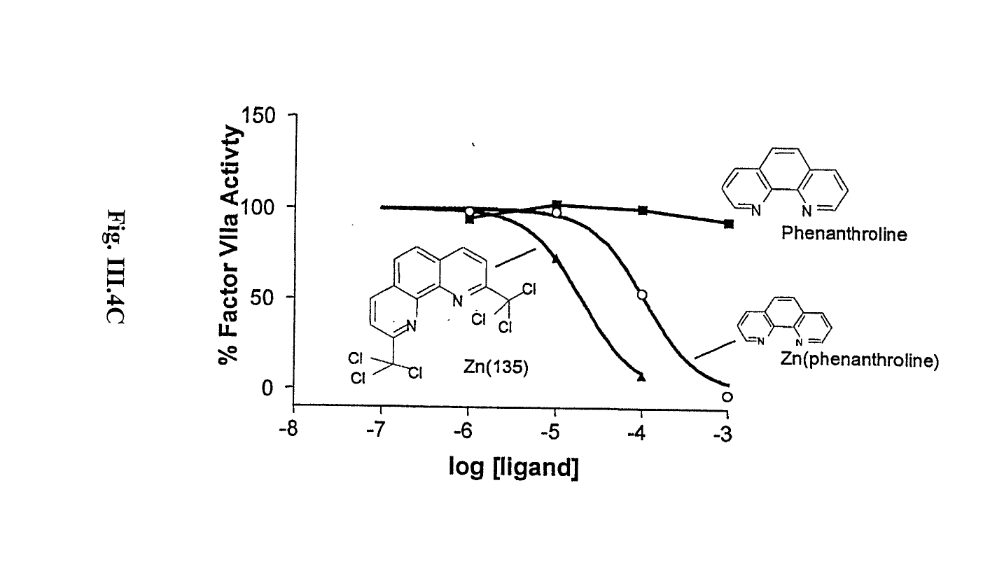

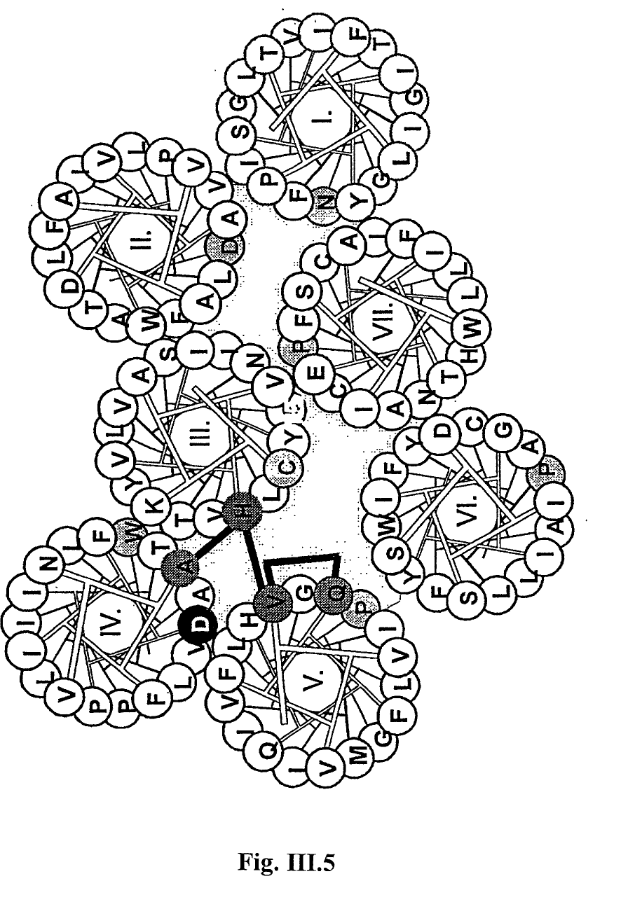

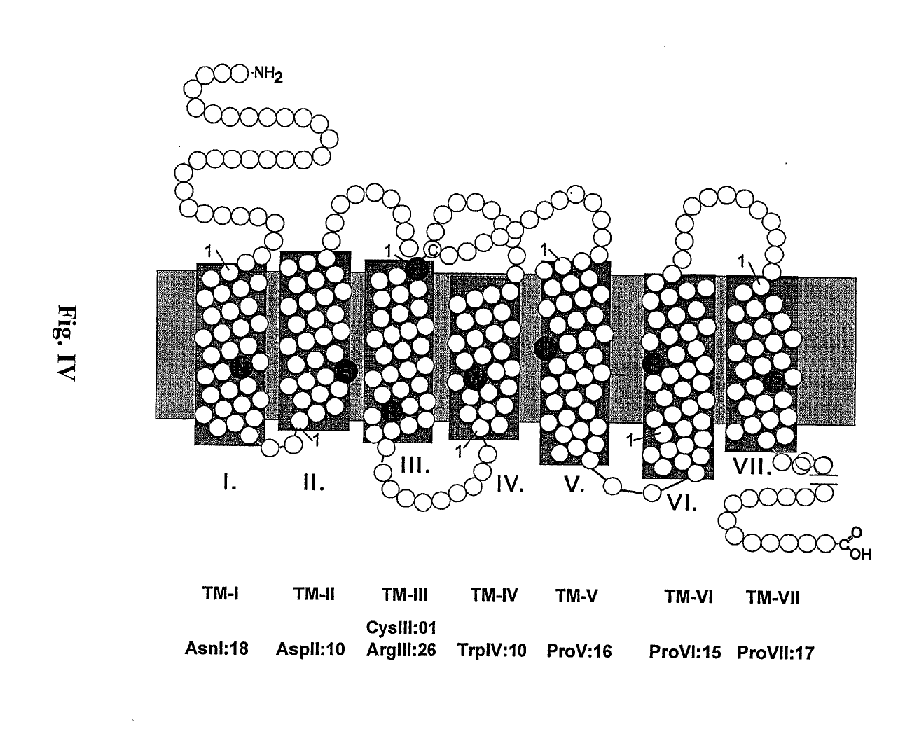

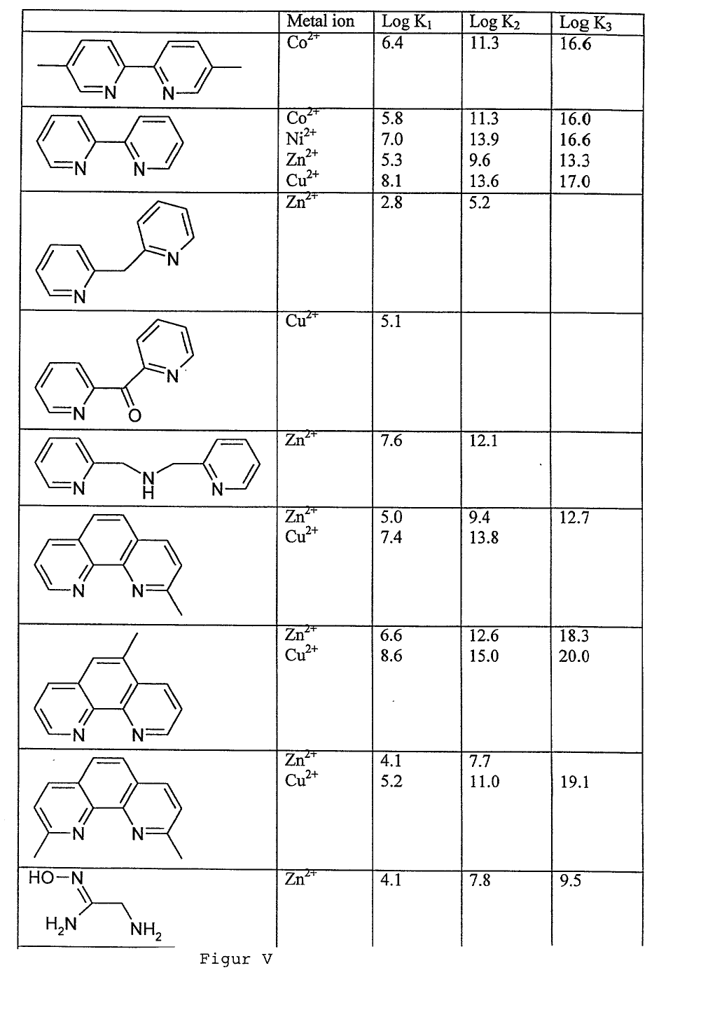

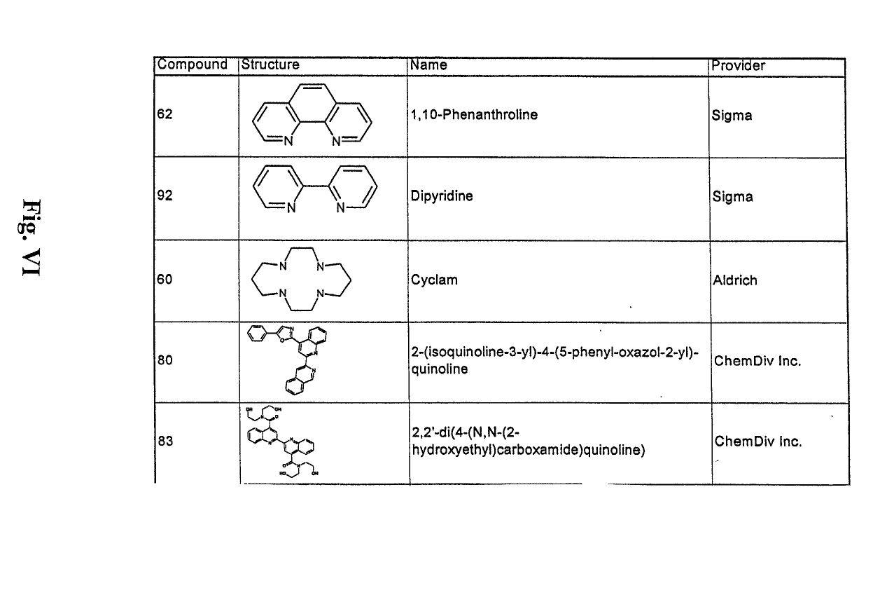

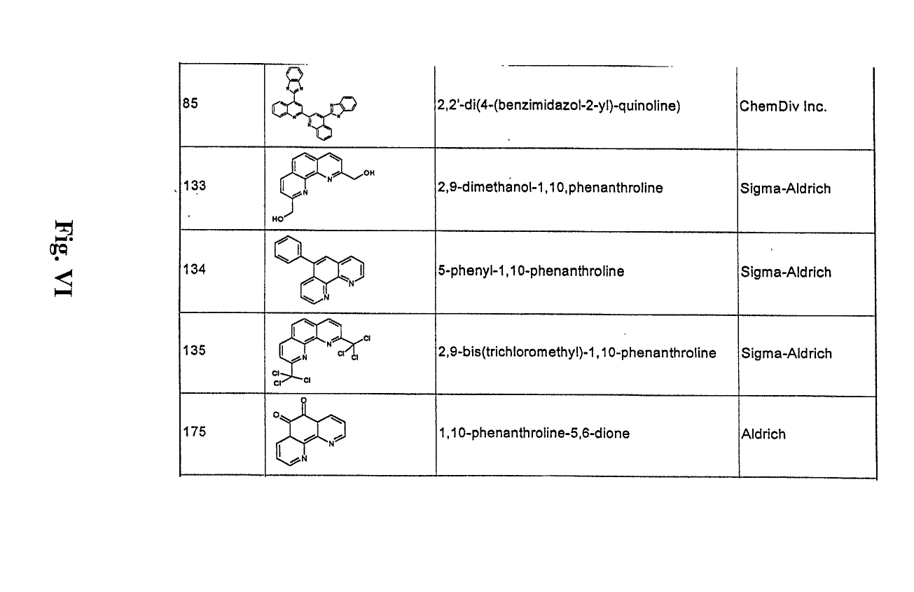

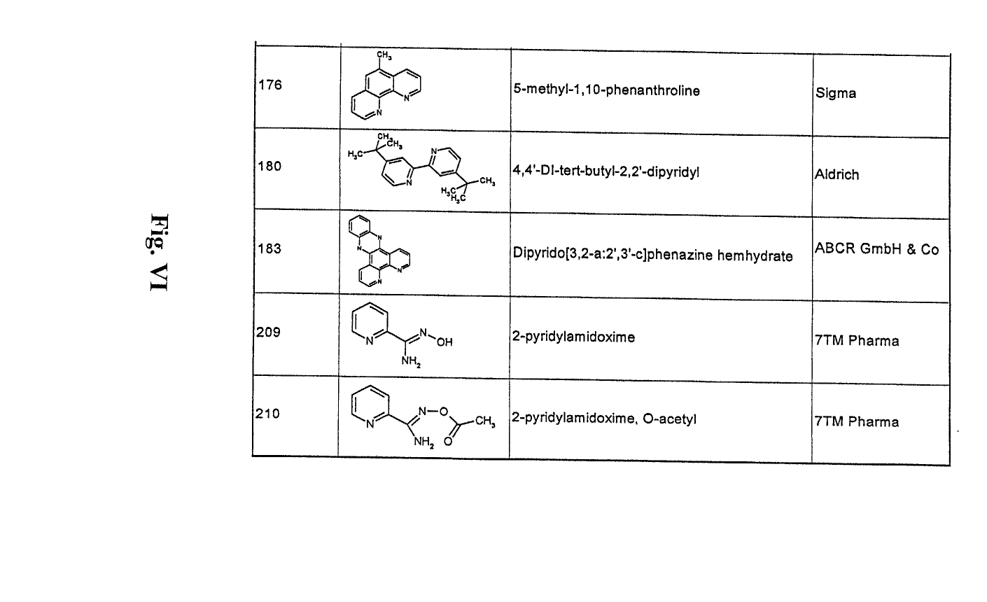

III.6 Structure-based optimisation of metal-ion chelators to use as antagonists in ‘pharmacological knock-out’ experiments. No FIG. 1

IV. OPTIMIZATION OF COMPOUNDS ON THE WILD-TYPE BIOLOGICAL TARGET MOLECULE No FIG. 1

APPENDIX—List of compounds which appear in the Examples.

1. A drug discovery process for identification of a small organic compound that is able to bind to a biological target molecule, the process comprising mutating a biological target molecule in such a way that at least one amino acid residue capable of binding a metal ion is introduced into the biological target molecule so as to obtain a metal ion binding site as an anchor point in the mutated biological target molecule.

2. A drug discovery process according to claim 1 further comprising
(a) contacting the mutated biological target molecule with a test compound which comprises a moiety including at least two heteroatoms for chelating a metal ion, under conditions permitting non-covalent binding of the test compound to the introduced metal ion binding site of the mutated biological target molecule, and
(b) detecting any change in the activity of the mutated biological target molecule or determining the binding affinity of the test compound to the mutated biological target molecule.

3. A drug discovery process according to claim 1 further comprising
(a) contacting the mutated biological target molecule with one or more members of a library of test compounds that comprise a moiety including at least two heteroatoms for chelating a metal ion, under conditions permitting non-covalent binding of at least a member of the library of test compounds to the introduced metal ion binding site of the mutated biological target molecule, and
(b) detecting any change in the activity of the mutated biological target molecule or determining the binding affinity of the test compound to the mutated biological target molecule.

4. A drug discovery process for identification of a small organic compound that is able to bind to a biological target molecule which has at least one metal ion binding site, the process comprising
(a) contacting the biological target molecule with a test compound which comprises a moiety including at least two heteroatoms for chelating a metal ion, under conditions permitting non-covalent binding of the test compound to the metal ion binding site of the biological target molecule, and
(b) detecting any change in the activity of the biological target molecule or determining the binding affinity of the test compound to the biological target molecule.

5. A drug discovery process for identification of a small organic compound that is able to bind to a biological target molecule which bas at least one metal ion binding site, the process comprising
(a) contacting the biological target molecule with one or more members of a library of test compounds that comprise a moiety including at least two heteroatoms for chelating a metal ion, under conditions permitting non-covalent binding of at least a member of the library of test compounds to the metal ion binding site of the biological target molecule, and
(b) detecting any change in the activity of the biological target molecule or determining the binding affinity of the test compound to the biological target molecule.

6. A drug discovery process according to any of claims 1-5 further comprising
(c) identifying the test compound that non-covalently binds to the biological target molecule.

7. A drug discovery process according to any of claims 1-6 further comprising
(d) selecting two or more test compounds capable of forming a non-covalent binding to a biological target molecule, and capable of changing the activity of the biological target molecule or the binding affinity of the test compound to the biological target molecule to form a library of test compounds.

8. A drug discovery process according to any of claims 1-3 or 6-7 further comprising
(e) contacting the biological target molecule in wild-type, non-mutated form with at least one test compound determined to non-covalently bind the mutated biological target molecule in step (a), and
(f) detecting any change in the activity of the biological target molecule or determining the binding affinity of the test compound to the biological target moelcule.

9. A drug discovery process according to any of claims 1-3 or 6-7 further comprising
(e) contacting the biological target molecule in wild-type, non-mutated form with two or more members of a library of test compounds, wherein the test compounds in chelated form have been determined to non-covalently bind the mutated biological target molecule in step (a), and
(f) detecting any change in the activity of the biological target molecule or determining the binding affinity of the test compound to the biological target moelcule.

10. A drug discovery process according to claims 8 or 9 further comprising
(g) identifying the test compound that interacts with the wild-type biological target molecule.

11. A drug discovery process according to any of claims 1-7 further comprising
(e) contacting the biological target molecule in wild-type, non-mutated form with at least one test compound determined to non-covalently bind the mutated or non-mutated biological target molecule in step (a) but lacking a metal ion chelated thereto, and
(f) detecting any change in the activity of the biological target molecule or determining the binding affinity of the non-chelated test compound to the biological target moelcule.

12. A drug discovery process according to any of claims 1-7 further comprising
(e) contacting the biological target molecule in wild-type, non-mutated form with two or more members of a library of non-chelated test compounds, wherein the test compounds in chelated form have been determined to non-covalently bind the mutated or non-mutated biological target molecule in step (a), and
(f) detecting any change in the activity of the biological target molecule or determining the binding affinity of the non-chelated test compound to the biological target moelcule.

13. A drug discovery process according to claims 11 or 12 further comprising
(g) identifying the non-chelated test compound that interacts with the wild-type biological target molecule.

14. A drug discovery process according to any of claims 8-13 further comprising
(a) identification of any binding or interaction between the non-chelated test compound and the wild-type biological target molecule.

15. A drug discovery process according to any of claims 1-14, wherein the biological target molecule is a protein.

16. A drug discovery process according to claims 15, wherein the protein comprises an amino acid residue and wherein the metal ion binding site in the protein is introduced by amino acid substitution at or in the vicinity of
1) a site where the binding of the test compound will interfere with the binding to another protein, for example a regulatory protein, or to a domain of the same protein;
2) a site where the binding of the test compound will interfere with the cellular targeting of the protein;
3) a site where the binding of the test compound will directly or indirectly interfere with the binding of substrate or the binding of an allosteric modulatory factor for the protein;
4) a site where the binding of the test compound may interfere with the intra-molecular interaction of domains within the protein, for example the interaction of a regulatory domain with a catalytic domain;
5) a site where binding of the test compound will interfere with the folding of the protein, for example the folding of the protein into its active conformation; or
6) a site where the binding of the test compound will control the activity of the protein, for example by an allosteric mechanism.

17. A drug discovery process according to any of the preceding claims, wherein the metal ion binding amino acid residue in the biological target molecule is introduced by site-directed mutagenesis.

18. A drug discovery process according to any of the preceding claims, wherein the mutated biological target molecule is obtained as a recombinant expression product in purified or non-purified form.

19. A drug discovery process according to any of the preceding claims, wherein the mutated biological target molecule is obtained as a synthetic or semi-synthetic product.

20. A drug discovery process according to claim 15, wherein step (a) in any of claims 2-5 comprises the further step of determining, based on the three-dimensional stucture of the specific protein in question or the primary structure of the specific protein together with a three-dimensional model of the class of proteins to which the specific protein belongs, the location of the metal ion binding amino acid residue in the mutated or non-mutated protein, and determining the location of at least one other amino acid residue in the vicinity of the metal ion binding amino acid residue.

21. A drug discovery process according to claim 15, wherein the binding of the test compound to the mutated or non-mutated protein in step (a) in any of claims 2-5 is determined using detection of any changes in the biological activity of the protein, competition with binding of a labelled ligand of the protein, or using a metal ion chelator which is in itself detectable or labelled with a detectable labelling agent.

22. A drug discovery process according to claim 19, wherein the amino acid residue in the vicinity of the metal ion binding amino acid residue is one which is capable of directly or indirectly binding at least one functional group of the test compound other than the metal ion.

23. A drug discovery process according to claim 22, wherein the amino acid residue capable of binding at least one functional group of the test compound other than the metal ion is detected using site-directed mutagenesis of at least one amino acid residue of the protein potentially involved in interaction with said functional group of the test compound other than the metal ion, followed by expression of the mutated protein in a suitable cell, contacting said cell or a portion thereof including the mutated protein with the test compound, and detecting any changes in the activity of the protein, determining any effect on binding in a competitive binding assay using a labelled ligand of the protein, or using a chelating agent which is in itself detectable or labelled with a detectable labelling agent.

24. A drug discovery process according to claim 22, wherein the amino acid residue capable of binding at least one functional group of the test compound other than the metal ion is detected by structural analysis employing i) a process involving crystallisation followed by X-ray, or ii) a process involving NMR.

25. A drug discovery process according to claim 15, wherein step (a) of any claims 2-5 comprises the further steps of improving the binding affinity of a metal ion chelate to the mutated or non-mutated protein, the method comprising
(i) selecting a metal ion chelate with an activity to or a binding affinity to the mutated protein of 50 μM or better as identified by the method of claim 21,
(ii) mapping the site of the protein to which the chelate binds using the method of claim 20, 23 and/or 24,
(iii) optionally locating at least one amino acid residue in the vicinity of the chelate,
(iv) altering one or more functional group of the chelate to optimise for direct or indirect interaction with said amino acid residue to generate a library of chelate derivatives,
(v) screening the derivatives of step (iv) by the method of claim 21,
(vi) selecting metal ion chelates having at least a two fold increase in activity or in binding affinity,
(vii) optionally repeating any one or a combination of two or more of steps (i)-(vi) one or more times to generate metal ion chelating compounds with an improved binding affinity for the mutated or non-mutated protein, and
(viii) optionally screening the thus selected metal ion chelates for binding to the wild-type protein by the method of claim 21,
(ix) optionally selecting metal ion chelates having at least an activity or a binding affinity to the wild-type protein of 50 μM or better as identified by the method of claim 21, and
(x) optionally repeating any one or a combination of two or more of steps (viii)-(ix) one or more times to generate metal ion chelating compounds with an improved binding affinity for the wild-type protein.

26. A drug discovery process according to claim 15, wherein step (e) in any of claims 8-12 comprises the further steps of generating a library of test compounds which are derivatives of a test compound found to interact with the wild-type protein in step (e), each test compound in the library being provided with at least one functional group for direct or indirect interaction with at least one amino acid of the wild-type protein, which functional group differs from at least one functional group of each of the other test compounds, and screening the test compound library for compounds interacting with the wild-type protein.

27. A drug discovery process according to claim 15, wherein step (e) in any of claims 8-12 is performed by detecting any changes in the activity of the protein, detecting an effect on binding in a competitive binding assay using a labelled ligand of the protein, or using a chelating agent which is in itself detectable or labelled with a detectable labelling agent.

28. A drug discovery process according to claim 15, wherein step (e) in any of claims 8-12 comprises the further step of determining—based on the three-dimensional structure of the specific protein in question or the primary structure of the specific protein together with the three-dimensional model of the class of proteins to which the specific protein belongs, and based on the information provided by the method of claim 25 of the location of amino acid residues in the vicinity of the metal ion binding residue introduced in the mutated protein—the location of an amino acid residue in the wild-type protein binding at least one functional group of a test compound.

29. A drug discovery process according to claim 28 wherein the amino acid residue capable of binding at least one functional group of the test compound is detected using site-directed mutagenesis of at least one amino acid residue of the wild-type protein potentially involved in interaction with said functional group of the test compound, followed by expression of the mutated protein in a suitable cell, contacting said cell or a portion thereof including the mutated protein with the test compound, and determining any effect on binding using detection of any changes in the biological activity of the protein, a competitive binding assay using a labelled ligand of the protein, or using a chelating agent which is in itself detectable or labelled with a detectable labelling agent.

30. A drug discovery process according to claim 28, wherein the amino acid residue capable of binding at least one functional group of the test compound other than the metal ion is detected by structural analysis employing i) a process involving crystallisation followed by X-ray, or ii) a process involving NMR.

31. A drug discovery process according to any of claims 1-14, wherein the biological target molecule is selected from the group consisting of proteins, polypeptides, oligopeptides, nucleic acids, carbohydrated, nucleoproteins, glycoproteins, glycolipids, lipoproteins and derivatives thereof.

32. A drug discovery process according to claim 31, wherein the biological target molecule is a protein selected from the group consisting of membrane receptors, signal transduction proteins, scaffolding proteins, nuclear receptors, steroid receptors, intracellular receptors, transcription factors, enzymes, allosteric enzyme regulator proteins, growth factors, hormones, neuropeptides or immunoglobulins.

33. A drug discovery process according to claim 32, wherein the protein is a membrane protein.

34. A drug discovery process according to claim 33, wherein the biological target molecule is a membrane protein and the metal ion binding site in the biological target molecule is introduced in a ligand binding crevice of the membrane protein.

35. A drug discovery process according to claim 33, wherein the membrane protein is an integral membrane protein.

36. A drug discovery process according to claim 35, wherein the membrane protein comprises 1-14 transmembrane domains such as 1, 2, 3, 4, 5, 6, 7, 8, 9, 10, 11, 12, 13 or 14 domains.

37. A drug discovery process according to claim 36, wherein the membrane protein is a receptor such as a tyrosine kinase receptor, e.g. a growth factor receptor such as the growth hormone, insulin, epidermal growth factor, transforming growth factor, erythropoietin, colony-stimulating factor, platelet-derived growth factor receptor or nerve growth factor receptor (TrkA or TrkB).

38. A drug discovery process according to claim 36, wherein the membrane protein is a purinergic ion channel.

39. A drug discovery process according to claim 36, wherein the membrane protein is a ligand-gated ion channel, such as a nicotinic acetylcholine receptor, GABA receptor, or glutamate receptor (NMDA or AMPA).

40. A drug discovery process according to claim 36, wherein the membrane protein is a voltage-gated ion channel, such as a potassium, sodium, chloride or calcium channel.

41. A drug discovery process according to claim 36, wherein the membrane protein is a 7TM receptor, a G-protein coupled receptor, such as the acetylcholine receptors, ACTH receptors, adenosine receptors, adrenoceptors, anaphylatoxin chemotactic receptor, angiotensin receptors, bombesin (neuromedin) receptors, bradykinin receptors, calcitonin and calcitonin gene related peptide receptors, conopressin receptors, corticotropin releasing factor receptors, amylin receptors, adrenomedullin receptors, calcium sensors, cannabinoid receptors, CC-chemokine receptors, cholecystokinin receptors, dopamine receptors, eicosanoid receptors, endothelin receptors, fMLP receptors, GABA_{B} receptors, galanin receptors, gastrin receptors, gastric inhibitory peptide receptors, glucagons receptors, gluragon-like I and II receptors, glutamate metabotropic receptors, glycoprotein hormone (e.g. FSH, LSH, TSH, LH) receptors, gonaotopin releasing hormone receptors, growth hormone releasing hormone receptors, growth hormone releasing peptide (Ghrelin) receptors, histamine receptors, 5-hydroxytryptamine receptors, leukotriene receptors, lysophospholipid receptors, melanocortin receptors, melanin concentrating hormone receptors, melatonin receptors, melanocortin receptors, neuropeptide Y receptors, neurotensin receptors, odor component receptors, opioid and opioid-like receptors, retinal receptors (opsins), orexin receptors, oxytocin receptors, parathyroid hormone and parathyroid hormone-related peptide receptors, P2Y receptors, pheromone receptors, platelet-activating factor receptors, prostanoid receptors, protease-activated receptors, secretin receptors, somatostatin receptors, tachykinin receptors, thyrotropin-releasing hormone receptors, pituitary adenylate activating peptide receptors, vasopressin receptors, vasoactive intestinal peptide receptors and virally encoded 7TM receptors; in particular galanin receptors, P2Y receptors, chemokine receptors, metabotropic glutamate receptors, melanocortin receptors, bombesin receptors, cannabinoid receptors, lysophospholipid receptors, fMLP receptors, neuropeptide Y receptors, tachykinin receptors, dopamine receptors, histamine receptors, 5-hydroxytryptamine receptors, histamine receptors, mas-proto-oncogene, acetylcholine, oxytocin, herpes virus encoded 7TM receptors, epstein-barr virus induced 7TM receptors, cytomegalovirus encoded receptors and bradykinin receptors; preferably galanin receptor type 1, leukotiene B4 receptor, CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, mGLU-R1, mGLU-R2, m-GLU-F3, m-GLU-R4, m-GLU-R5, m-GLU-R6, m-GLU-R8, melanin concentration hormone receptors, melanocortin-1 receptor, melanocortin-3 receptor, melanocortin-4 receptor, melanocortin-5 receptor, bombesin receptor subtype 3, cannabinoid receptor 1, cannabinoid receptor 2, EDG-2, EDG-4, FMLP-related receptor I, FMLP-related receptor II, NPY Y6 receptor, NPY Y5 receptor, NPY Y4 receptor, NK-1 receptor, NK-3 receptor, D2 receptor (short), D2 receptor (long), Duffy antigen, U27, U28, UL33 and U78 from human cytomegalovims, U12 and, U51 from human herpes virus 6A, 6B or 7, ORF-74 from human herpes virus 8, Epstein Barr virus induced receptor-2, histamine H1 receptor, MAS proto-oncogene, muscarinic M1 receptor, muscarinic M2 receptor, muscarinic M3 receptor, muscarinic M5 receptor, oxytocin receptor, XCR1 receptor, RDC1 receptor, GPR12 receptor or GPR3 receptor.

42. A drug discovery process according to claim 36, wherein the membrane protein is a transporter protein, such as a GABA, monoamine, glutaminic acid or nucleoside transporter.

43. A drug discovery process according to claim 36, wherein the membrane protein is a multidrug resistance protein, e.g. a P-glycoprotein, multidrug resistance associated protein, drug resistance associated protein lung resistance related protein breast cancer resistance protein, adenosine triphosphate-binding cassette protein Byr, QacA or EmrAB/TolC pump.

44. A drug discovery process according to claim 36, wherein the membrane protein is a cell adhesion molecule, e.g. NCAM, VCAM or ICAM.

45. A drug discovery process according to claim 36, wherein the membrane protein is an enzyme such as adenylyl cyclase.

46. A drug discovery process according to claim 35, wherein the membrane protein is an orphan receptor.

47. A method of identifying a metal ion binding site in a biological target molecule, the method comprising
(a) contacting the biological target molecule with a test compound which comprises a moiety including at least two heteroatoms for chelating a metal ion, under conditions permitting non-covalent binding of the test compound to the biological target molecule, and
(b) detecting any change in the activity of the biological target molecule or determining the binding affinity of the test compound to the biological target molecule.

48. A method of identifying a metal ion binding site in a protein, the method comprising
(a) analysing the nucleotide sequence of the gene coding for the protein in order to deduce the amino acid sequence,
(b) building a molecular model of the protein or a part of the protein based on the deduced amino acid sequence and the generic three-dimensional model of the class of proteins to which the specific protein belongs,
(c) identifying the presence of amino acid residues such as, e.g., histidine, cysteine and/or aspartate residues, capable of binding a metal ion and located in suitable relative positions.

49. A method according to claim 47 or 48, wherein the test compound is contacted with two or more biological target molecules for identification of possible metal ion binding sites thereof.

50. A method of identifying a metal ion binding site in a protein, the method comprising
(a) selecting a nucleotide sequence suspected of coding for a protein and deducing the amino acid sequence thereof,
(b) expressing said nucleotide sequence in a suitable host cell,
(c) contacting said cell or a portion thereof including the expressed protein with a test compound which comprises a moiety including at least two heteroatoms for chelating a metal ion, under conditions permitting non-covalent binding of the test compound to the protein, and detecting any change in the activity of the protein or determining the binding affinity of the test compound to the protein, and
(d) determining, based on the generic three-dimensional model of the class of proteins to which the protein or suspected protein belongs, at least one metal ion binding amino acid residue located in said protein to locate the metal ion binding site of said protein.

51. A method of mapping a metal ion binding site of a protein, the method comprising
(a) contacting the protein with a test compound which comprises a moiety including at least two heteroatoms for chelating a metal ion, under conditions permitting non-covalent binding of the test compound to the protein, and detecting any change in the activity of the protein or determining the binding affinity of the test compound to the protein, and
(b) determining, based on the primary structure of the specific protein in question and the generic three-dimensional model of the class of proteins to which the specific protein of step (a) belongs, at least one metal ion binding amino acid residue located in the membrane protein to identify the metal ion binding site of said membrane protein.

52. A drug discovery process according to any of claims 1-46 further comprising a method of any of claims 47-51.

53. A drug discovery process according to any of the preceding claims, wherein the test compound has a log K value in a range of from about 3 to about 18 such as, e.g. from about 3 to about 15, from about 3 to about 12, from about 4 to about 10, from about 4 to about 8, from about 4.5 to about 7, from about 5 to about 6.5 such as from about 5.5 to about 6.5.

54. A drug discovery process according to any of the preceding claims, wherein the test compound forms a chelate with a metal ion selected from the group consisting of Co, Cu, Ni, Pt and Zn including the various oxidation steps such as, e.g., Co (II), Co (III), Cu (I), Cu (II), Ni (II), Ni (III), Pt (II), Pt (IV) and Zn (II).

55. A drug discovery process according to any of the preceding claims, wherein the test compound has at least two heteroatoms, similar or different, selected from the group consisting of nitrogen (N), oxygen (O), sulfur (S), selenium (Se) and phosphorous (P).

56. A drug discovery process according to any of the preceding claims, wherein the test compound has the general formula I 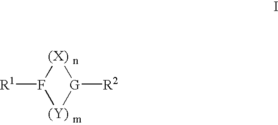
wherein F is N, O, S, Se or P; and G is N, O, S, Se or P;
at least one of (X)ₙ and (Y)ₘ is present and if n is 0, then —(X)ₙ— is absent, and if m is 0, then —(Y)ₘ— is absent, and both n and m are not 0;
R¹ and R², which are the same or different, are radicals preferably selected from the group consisting of hydrogen, a C₁-C₁₅ alkyl, C₂-C₁₅ alkenyl, C₂-C₁₅ alkynyl, aryl, cycloallyl, alkoxy, ester, —OCOR′, —COOR′, heteroalkyl, heteroalkenyl, heteroalkynyl, heterocycloalkyl, heterocycloalkenyl, heterocycloalkynyl or heteroaryl group, an amine, imine, nitro, cyano, hydroxyl, alkoxy, ketone, aldelhyde, carboxylic acid, thiol, amide, sulfonate, sulfonic acid, sulfonamide, phosphonate, phosphonic acid group or a combination thereof, optionally substituted with one or more substituents selected from the same group as R¹ and/or a halogen such as F, Cl, Br or I;
R′ is hydrogen, alkyl, substituted alkyl, alkenyl, substituted alkenyl, alkynyl, substituted alkynyl, aryl, substituted aryl, arylalkyl, substituted arylalkyl, heteroalkyl, substituted heteroalkyl, heteroalkenyl, substituted heteroalkenyl, heteroalkynyl, heteroaryl, substituted heteroaryl, cycloalkyl, substituted cycloalkyl, cycloalkenyl, substituted cycloalkenyl, heterocycloalkyl, substituted heterocycloalkyl, heterocycloalkenyl or substituted heterocycloalkenyl;
R¹ and/or R² optionally forming a fused ring together with any of F, (X)ₙ or a part of (X)ₙ G, (Y)ₘ or a part of (Y)ₘ or R¹ and R² themselves forming a fused ring;
X and Y are the same or different and have the same meaning as R′ such as, e.g., —CH₂—, —CH₂—CH₂—, —CH₂—S—CH₂—, —CH₂—N—CH₂—, —CH═CH—CH═CH—, —(CH₂)_{d}—(Z)ₑ(V)_{f}—(W)_{g}—(CH₂)ₕ—, —CH₂—O—CH₂—, wherein each of Z and W are independently C, S, O, N, Se or P and V is —CH— or —CH₂—;
(X)ₙ and/or (Y)ₘ optionally being substituted with one or more substituents selected from the same group as R¹ and/or a halogen such as F, Cl, Br or I;
n is 0 or an integer of 1-5,
m is 0 or an integer of 1-5,
e and/or g are an integer of 1-3,
d, f and/or h are an integer of 1-7.

57. A drug discovery process according to claim 56, wherein the test compound has the general formula II 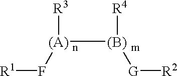
wherein F, G, R¹ and R² have the same meaning as in claim 56, R³ and R⁴ have the same meaning as R¹ and/or R², and A and B have independently the same meaning as X and Y in formula I, n and m have the same meaning as in formula I except that n and m may be 0 at the same time and then the basic structure is
R¹—F—G—R²
and when n or m are 0, respectively, then the basic structures of formula II are

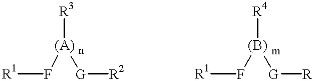

58. A drug discovery process according to claim 57, wherein F and/or G is nitrogen (N) and/or oxygen (O) and the test compound has the general formula III, IV, V, VI or VII: 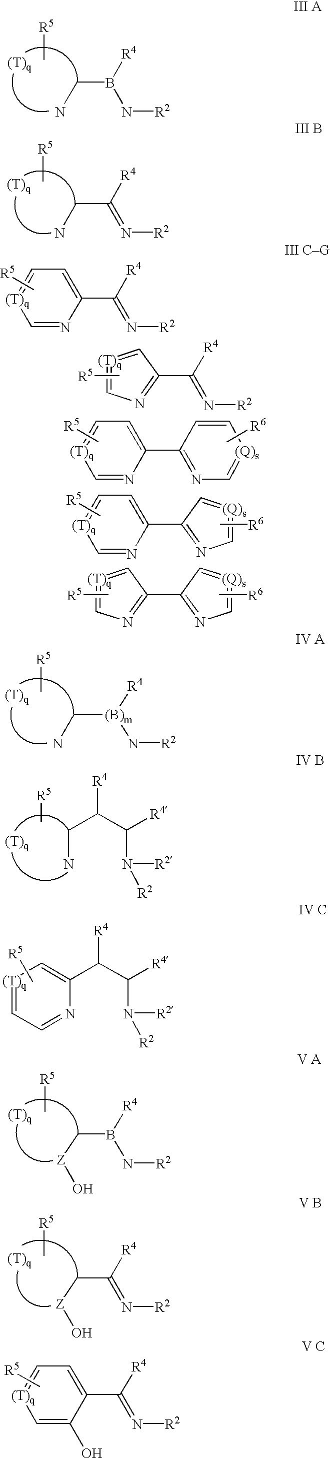
wherein T and Q are heteroatoms, and q and s independently are 0 or an integer of from 1 to 4;
the meanings of q and s for q and/or s being 0 are the same as in Formula II for n and m;
a circle indicates a fused alkyl, alkenyl, aryl, heteroalkyl, heteroalkenyl, heteroalkynyl or heteroaryl ring having from 3-7 atoms in the ring;
R⁵ has the same meaning as R¹ and/or R²;
and in Formulas III C-G, IV C and V C-D, T and/or Q may be placed anywhere in the cyclic system.

59. A drug discovery process according to claim 58, wherein the test compound has the general formula VIII 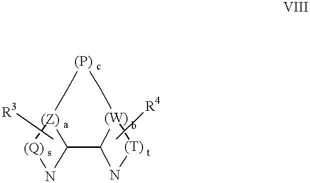
wherein R³, R⁴, Z, W and P are as defined herein before, a and/or b are an integer of 1-7 and c is 0 or an integer of 1-7, and each of Q and T is independently —CH— or —CH₂—, s is an integer of 1-7, and t is an integer of 1-7, are believed to be particularly suitable; when c is 0 in the above Formula VIII then —(P)_{c}— is absent, i.e. there is no bond between (Z)ₐ and (W)_{b}.

60. A drug discovery process according to claim 56, wherein the test compound as the general formula IX 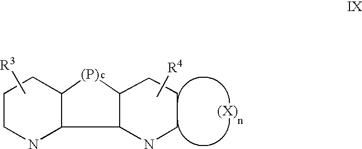
wherein R³, R⁴, P, X and n are as indicated above, and r is 0 or an inter of 1-3, and when r is 0 then —(P)ᵣ— is absent.

61. A drug discovery process according to claim 56, wherein the test compound has the general formula X 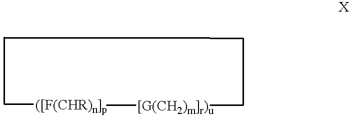
wherein F is N, O or S and G is N, O or S,
n is an integer from 1 to 5,
m is 0 or an integer from 1 to 5,
p and/or r are 0 or an integer from 1 to 8,
u is an integer from 1 to 8, and
R has the same meaning as R¹ in Formula I.

62. A drug discovery process according to claim 56, wherein the test compound has the general formula XI 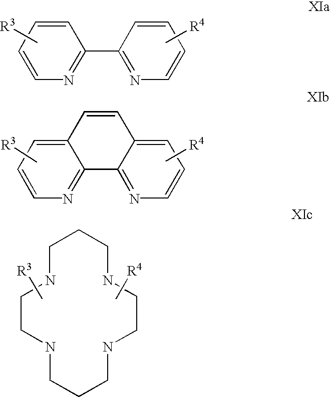
wherein R³ and R⁴ are as indicated above in formula I.

63. A drug discovery process according to any of claims 53-62, wherein the metal ion is one that binds to an amino acid residue containing a S, O, N, Se and/or P atom or with an aromatic amino acid residue.

64. A drug discovery process according to claim 63, wherein the amino acid residue is selected from the group consisting of Ser, Lys, Arg, Tyr, Thr, Trp, Phe, Asp, Glu, Asn, Gin, Cys and His, in particular Asp, Glu, Cys and His, preferably His.

65. A drug discovery process according to any of claims 53-64, wherein the metal ion is selected from the group consisting of aluminium, antimony, arsenic, astatine, barium, beryllium, bismuth, boron, cadmium, calcium, cerium, cesium, chromium, cobalt, copper, dysprosium, erbium, europium, gadolinium, gallium, germanium, gold, hafnium, holmium, indium, iridium, iron, lanthanum, lead, lutetium, magnesium, manganese, mercury, molybdenum, neodymium, nickel, niobium, osmium, palladium, platinum, polonium, praseodymium, promethium, rhenium, rhodium, rubidium, ruthenium, samarium, scandium, selenium, silicon, silver, strontium, tantalum, technetium, tellurium, terbium, thallium, thorium, thulium, tin, titanium, tungsten, vanadium, ytterbium, yttrium, zinc, zirconium, and oxidation states and isotopes thereof, in particular aluminium, antimony, barium, bismuth, calcium, chromium, cobalt, copper, europium, gadolinium, gallium, germanium, gold, indium, iron, lutetium, manganese, magnesium, nickel, osmium, palladium, platinum, rhenium, rhodium, rubidium, ruthenium, samarium, silver, strontium, technetium, terbium, thallium, thorium, tin, yttrium, zinc, and oxidation states or isotopes thereof; in particular cobalt, copper, nickel, platinum, ruthenium, and zink, and oxidation states and isotopes thereof, preferably calcium (II), cobalt (II) and (III), copper (I) and (II), europium (III), iron (II) and (III), magnesium (II), manganese (II), nickel (II) and (III), palladium (II), platinum (II) and (V), ruthenium (II), (III), (IV), (VI) and (VIII), samarium (III), terbium (III), zinc (II), or isotopes thereof, preferably cobalt (II) and (III), copper (I) and (II), nickel (II) and (III), zinc (II) and platinum (II) and (V), or isotopes thereof.

66. A drug discovery process according to any of claims 53-65, wherein the test compound is a chelate like e.g. metal ion-phenanthroline complex, metal ion bipyridyl complex and metal ion-1,4,8,11-tetraazacyclotetradecane complex such as, e.g., a Cu²⁺-phenanthroline complex, a Zn²⁺-phenanthroline complex, a Cu²⁺-bipyridyl complex, a Zn²⁺-bipyridyl complex, a Ca²⁺-bipyridyl complex, a Cu²⁺-1,4,8,11-tetraacyclotetradecane, a Zn²⁺-1,4,8,11-tetraazacyclotetradecane.

67. A chemical library comprising a plurality of test compounds of the following general formula I 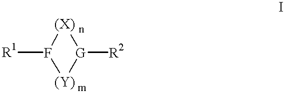
wherein F is N, O, S, Se or P; and G is N, O, S, Se or P;
at least one of (X)ₙ and (Y)ₘ is present and if n is 0, then —(X)ₙ— is absent, and if m is 0, then —(Y)ₘ— is absent, and both n and m are not 0;
R¹ and R², which are the same or different, are radicals preferably selected from the group consisting of: hydrogen, a C₁-C₁₅ alkyl, C₂-C15 alkenyl, C₂-C₁₅ alkynyl, aryl, cycloalkyl, alkoxy, ester, —OCOR′, —COOR′, heteroalkyl, heteroalkenyl, heteroalkynyl, heterocycloalkyl, heterocycloalkenyl, heterocycloalkynyl or heteroaryl group, an amine, imine, nitro, cyano, hydroxyl, alkoxy, ketone, aldelhyde, carboxylic acid, thiol, amide, sulfonate, sulfonic acid, sulfonamide, phosphonate, phosphonic acid group or a combination thereof, optionally substituted with one or more substituents selected from the same group as R¹ and/or a halogen such as F, Cl, Br or I;
R′ is hydrogen, alkyl, substituted allyl, alkenyl, substituted alkenyl, alkynyl, substituted alkynyl, aryl, substituted aryl, arylalkyl, substituted arylalkyl, heteroalkyl, substituted heteroalkyl, heteroalkenyl, substituted heteroalkenyl, heteroalkynyl, heteroaryl, substituted heteroaryl, cycloalkyl, substituted cycloalkyl, cycloalkenyl, substituted cycloalkenyl, heterocycloalkyl, substituted heterocycloalkyl, heterocycloalkenyl or substituted heterocycloalkenyl;
R¹ and/or R² optionally forming a fused ring together with any of F, (X)ₙ or a part of (X)ₙ G, (Y)ₘ or a part of (Y)ₘ or R¹ and R² themselves forming a fused ring;
X and Y are the same or different and have the same meaning as R′ such as, e.g. —CH₂—, —CH₂—CH₂—, —CH₂—S—CH₂—, —CH₂—NCH₂—, —CH═CH—CH═H—, —(CH₂)_{d}—(Z)ₑ—(V)_{f}—(W)_{g}—(CH₂)ₕ—, —CH₂—O—CH₂—, wherein each of Z and W are independently C, S, O, N, Se or P and
V is —CH— or —CH₂—;
(X)ₙ and/or (Y)ₘ optionally being substituted with one or more substituents selected from the same group as R¹ and/or a halogen such as F, Cl, Br or I;
n is 0 or an integer of 1-5,
m is 0 or an integer of 1-5,
e and/or g are an integer of 1-3,
d, f and/or h are an integer of 1-7,
the test compounds being in the form of chelates formed between the test compound and a metal ion or atom selected from the group consisting of aluminium, antimony, arsenic, astatine, barium, beryllium, bismuth, boron, cadmium, calcium, cerium, cesium, chromium, cobalt, copper, dysprosium, erbium, europium, gadolinium, gallium, germanium, gold, hafnium, holmium, indium, indium, iron, lanthanum, lead, lutetium, magnesium, manganese, mercury, molybdenum, neodymium, nickel, niobium, osmium, palladium, platinum, polonium, praseodymium, promethium, rhenium, rhodium, rubidium, ruthenium, samarium, scandium, selenium, silicon, silver, strontium, tantalum, technetium, tellurium, terbium, thallium, thorium, thulium, tin, titanium, tungsten, vanadium, ytterbium, yttrium, zinc, zirconium, and oxidation states and isotopes thereof; in particular aluminium, antimony, barium, bismuth, calcium, chromium, cobalt, copper, europium, gadolinium, gallium, germanium, gold, indium, iron, lutetium, manganese, magnesium, nickel, osmium, palladium, platinum, rhenium, rhodium, rubidium, ruthenium, samarium, silver, strontium, technetium, terbium, thallium, thorium, tin, yttrium, zinc, and oxidation states or isotopes thereof, in particular cobalt, copper, nickel, platinum, ruthenium, and zink, and oxidation states and isotopes thereof, preferably calcium (II), cobalt (II) and (III), copper (I) and (II), europium (III), iron (II) and (III), magnesium (II), manganese (II), nickel (II) and (III), palladium (II), platinum (II) and (V), ruthenium (II), (III), (IV), (VI) and (VI), samarium (III), terbium (III), zinc (II), or isotopes thereof, preferably cobalt (II) and (III), copper (I) and (II), nickel (II) and (III), zinc (II) and platinum (II) and (V), or isotopes thereof.

68. A chemical library comprising a plurality of test compounds of the following general formula I 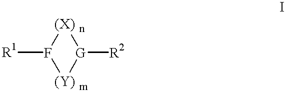
wherein F is N, O, S, Se or P; and G is N, O, S, Se or P;
at least one of (X)ₙ and (Y)ₘ is present and if n is 0, then —(X)ₙ— is absent, and if m is 0, then —(Y)ₘ— is absent, and both n and m are not 0;
R¹ and R², which are the same or different, are radicals preferably selected from the group consisting of: hydrogen, a C₁-C₁₅ alkyl, C₂-C₁₅ alkenyl, C₂-C₁₅ alkynyl, aryl, cycloalkyl, alkoxy, ester, —OCOR′, —COOR′, heteroalkyl, heteroalkenyl, heteroalkynyl, heterocycloalkyl, heterocycloalkenyl, heterocycloalkynyl or heteroaryl group, an amine, imine, nitro, cyano, hydroxyl, alkoxy, ketone, aldelhyde, carboxylic acid, thiol, amide, sulfonate, sulfonic acid, sulfonamide, phosphonate, phosphonic acid group or a combination thereof, optionally substituted with one or more substituents selected from the same group as R¹ and/or a halogen such as F, Cl, Br or I;
R′ is hydrogen, alkyl, substituted alkyl, alkenyl, substituted alkenyl, alkynyl, substituted alkynyl, aryl, substituted aryl, arylalkyl, substituted arylalkyl, heteroalkyl, substituted heteroalkyl, heteroalkenyl, substituted heteroalkenyl, heteroalkynyl, heteroaryl, substituted heteroaryl, cycloalkyl, substituted cycloalkyl, cycloalkenyl, substituted cycloalkenyl, heterocycloalkyl, substituted heterocycloalkyl, heterocycloalkenyl or substituted heterocycloalkenyl;
R¹ and/or R² optionally forming a fused ring together with any of P, (X)ₙ or a part of (X)ₙ G, (Y)ₘ or a part of (Y)ₘ or R¹ and R² themselves forming a fused ring;
X and Y are the same or different and have the same meaning as R′ such as, e.g., —CH₂—, —C₁₂—CH₂—, —CH₂—S—CH₂—, —CH₂—N—CH₂—, —CH═CH—CH═CH—, —(CH₂)_{d}—(Z)ₑ——(V)_{f}(W)_{g}(CH₂)ₕ—, —CH₂—O—CH₂—, wherein each of Z and W are independently C, S, O, N, Se or P and
V is —CH— or —CH₂—;
(X)ₙ and/or (Y)ₘ optionally being substituted with one or more substituents selected from the same group as R¹ and/or a halogen such as F, Cl, Br or I;
n is 0 or an integer of 1-5,
m is 0 or an integer of 1-5,
e and/or g are an integer of 1-3,
d, f and/or h are an integer of 1-7,
the test compounds being in non-chelated form.

69. A chemical library comprising a plurality of metal ions selected from the group consisting of aluminium, antimony, arsenic, astatine, barium, beryllium, bismuth, boron, cadmium, calcium, cerium, cesium, chromium, cobalt, copper, dysprosium, erbium, europium, gadolinium, gallium, germanium, gold, hafnium, holmium, indium, iridium, iron, lanthanum, lead, lutetium, magnesium, manganese, mercury, molybdenum, neodymium, nickel, niobium, osmium, palladium, platinum, polonium, praseodymium, promethium, rhenium, rhodium, rubidium, ruthenium samarium, scandium, selenium, silicon, silver, strontium, tantalum, technetium, tellurium, terbium, thallium, thorium, thulium, tin, titanium, tungsten, vanadium, ytterbium, yttrium, zinc, zirconium, and oxidation states and isotopes thereof; in particular aluminium, antimony, barium bismuth, calcium, chromium, cobalt, copper, europium, gadolinium, gallium, germanium, gold, indium, iron, lutetium, manganese, magnesium, nickel, osmium, palladium, platinum, rhenium, rhodium, rubidium, ruthenium, samarium, silver, strontium, technetium, terbium, thallium thorium, tin, yttrium, zinc, and oxidation states or isotopes thereof; in particular cobalt, copper, nickel, platinum ruthenium, and zink and oxidation states and isotopes thereof, preferably calcium (II), cobalt (II) and (III), copper (I) and (II), europium (III), iron (II) and (III), magnesium (II), manganese (II), nickel (II) and (III), palladium (II), platinum (II) and (V), ruthenium (II), (III), (IV), (VI) and (VIII), samarium (III), terbium (III), zinc (II), or isotopes thereof preferably cobalt (II) and (III), copper (I) and (II), nickel (II) and (III), zinc (II) and platinum (II) and (V), or isotopes thereof.

70. A chemical library according to claim 67 or 68, wherein the molecular weight of the individual test compounds is at the most 2000, log P is at the most 7, the number of hydrogen bond donors is at the most 10 and the number of hydrogen bond acceptors is at the most 15.

71. A chemical library according to claim 70, wherein the molecular weight of the individual test compounds is at the most 1500 such as, e.g., at the most 1000 or at the most 500; log P is at the most 6 such as, e.g., at the most 5; the number of hydrogen bond donors is at the most 8 such as, e.g., at the most 7, 6 or 5; and the number of hydrogen bond acceptors is at the most 13 such as, e.g., 12, 11 or 10

72. A chemical library according to any of claims 67-71 for use in a drug discovery process according to any of claims 1-52.

73. Use of a test compound according to any of claims 53-66 in chelated form as either a stabilizing or as a destabilizing agent for di- or oligomerisation of a biological target molecule.

74. Use according to claim 73, wherein the biological target molecule is a membrane protein.

75. Use according to claim 74, wherein the membrane protein is 7TM.

76. Use of a test compound according to any of claims 53-66 in pharmacological knock-out experiments employing a biological target molecule in which a silent metal ion binding site has been created without affecting the binding action of an endogenous ligand for the biological target molecule with an aim of determining the effect of either an agonist or an antagonist on the physiological function of the metal ion site engineered receptor introduced into an animal by homologous gene replacement.

77. A method for characterising an orphan receptor, the method comprising
(a) mutating the orphan receptor in such a way that at least one amino acid residue capable of binding a metal ion is introduced into the orphan receptor so as to obtain a metal ion binding site as an anchor point in the mutated orphan receptor,
(b) contacting the mutated orphan receptor with a test compound which comprises a moiety including at least two heteroatoms for chelating a metal ion, under conditions permitting non-covalent binding of the test compound to the introduced metal ion binding site of the orphan receptor, and
(c) monitoring the binding of the test compound to the mutated orphan receptor by e.g. functional assays or through ligand binding assays.

78. A method according to claim 77 further comprising an optimization step in order to improve the affinity of the test compound.

79. Use of a test compound according to any of claims 53-66 as tracers in binding assays for orphan receptors.
